(19) United States
(12) Patent Application Publication
(10) Pub. No.: US 2022/0378936 A1
(43) Pub. Date: Dec. 1, 2022

(54) DELIVERY SYSTEM COMPLEXES COMPRISING A PRECIPITATE OF AN ACTIVE AGENT AND METHODS OF USE

(71) Applicant: THE UNIVERSITY OF NORTH CAROLINA AT CHAPEL HILL, Chapel Hill, NC (US)

(72) Inventors: Leaf HUANG, Durham, NC (US); Jianfeng GUO, Chapel Hill, NC (US)

(21) Appl. No.: 17/767,816

(22) PCT Filed: Oct. 13, 2020
(86) PCT No.: PCT/US20/55424

§ 371 Date: Apr. 8, 2022

Related U.S. Application Data
(60) Provisional application No. 62/913,471, filed on Oct. 10, 2019.

(51) Int. Cl. A61K 47/69; A61K 31/555; A61K 47/54; A61K 47/68

(52) U.S. Cl. CPC A61K 47/6911; A61K 31/555; A61K 47/543; A61K 47/6803

(57) ABSTRACT

Provided herein are methods and compositions for the delivery of a combination of oxiplatin and folinic acid to a cell, tissue, or physiological site. The compositions comprise delivery system complexes comprising liposomes encapsulating dihydrate(1,2-diaminocyclohexane)platinum(II)-folinic acid or delivery system complexes comprising a 5-fluorouracil active metabolite. Also provided herein are methods for the treatment of cancer, wherein the methods comprise administering the delivery system complexes comprising dihydrate(1,2-diaminocyclohexane)platinum(II)-folinic acid or delivery system complexes comprising a 5-fluorouracil active metabolite that have therapeutic activity against the cancer.

### FEDERALLY SPONSORED RESEARCH OR DEVELOPMENT

This invention was made with government support under Grant Number CA198999 awarded by the National Institutes of Health. The government has certain rights in the invention.

### FIELD

The present invention involves the delivery of bioactive compounds using lipid-comprising delivery system complexes.

### BACKGROUND

The FOLFOX, a three-agent combination of folinic acid (FnA), 5-fluorouracil (5-Fu) and oxaliplatin (OxP), has been used in the treatment of colorectal cancer (CRC) for decades. Despite the improved survival, patients still suffer from the drawbacks such as toxicity, high cost, and long course of treatment.

Therefore, new strategies to address these issues are needed to further provide clinical benefits. The subject matter described herein addresses these and other shortcomings of currently available FOLFOX treatment modalities, in part, by improving safety and efficacy.

### BRIEF SUMMARY

In certain embodiments, the subject matter described herein is directed to a compound having the structure:

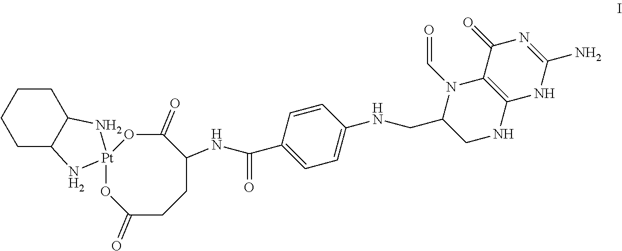

In certain embodiments, the subject matter described herein is directed to delivery system complexes comprising the compound of Formula I. In certain embodiments, the subject matter described herein is directed to a pharmaceutical composition comprising the compound of Formula I and a pharmaceutically acceptable excipient.

In certain embodiments, the subject matter described herein is directed to delivery system complexes comprising the compound of Formula I, wherein the delivery system complex comprises a liposome-encapsulated compound of Formula I.

In certain embodiments, the subject matter described herein is directed to delivery system complexes comprising the compound of Formula I, wherein the delivery system complex comprises a liposome-encapsulated compound of Formula I, wherein the liposome comprises a lipid bilayer.

In certain embodiments, the subject matter described herein is directed to methods of treatment of cancer, wherein the method comprises administering to a subject, a compound of Formula I or a delivery system complex comprising a compound of Formula I. In certain embodiments, these methods further comprise administering an antimetabolite drug, such as 5-fluorouracil (5-Fu) or a nanoformulation containing FdUMP (5-Fu active metabolite), i.e. Nano-FdUMP.

In certain embodiments, the subject matter described herein is directed to a delivery system complex comprising, a first type of stabilized single-lipid layer core comprising an anti-metabolite complex, a second type of stabilized single-lipid layer core comprising the compound of Formula I, wherein the cores are encapsulated by a polymer, such as PLGA, PLGA-PEG or PLGA-PEG-AEAA.

In certain embodiments, the subject matter described herein is directed to a delivery system complex comprising, a first type of stabilized single-lipid layer core comprising an anti-metabolite complex, a second type of stabilized single-lipid layer core comprising the compound of Formula I, and irinotecan (SN-38), wherein the cores and SN-38 are encapsulated by a polymer, such as PLGA, PLGA-PEG or PLGA-PEG-AEAA.

In certain embodiments, the subject matter described herein is directed to methods of treatment of cancer, wherein the method comprises administering to a subject, a delivery system complex comprising, a core comprising an anti-metabolite complex, said anti-metabolite complex comprising a 5-fluorouracil active metabolite, wherein said core is encapsulated by a liposome.

In certain embodiments, the subject matter described herein is directed to a delivery system complex comprising, a core comprising an anti-metabolite complex, said anti-metabolite complex comprising a 5-fluorouracil active metabolite, wherein said core is encapsulated by a liposome.

In certain embodiments, the delivery system complexes can comprise a targeting ligand and are referred to as targeted delivery system complexes. These targeted delivery system complexes target diseased cells, enhancing the effectiveness and minimizing the toxicity of the delivery system complexes.

In certain embodiments, the subject matter described herein is directed to methods of preparing a compound of Formula I or a delivery system complex comprising a compound of Formula I.

These and other embodiments are described herein.

### BRIEF DESCRIPTION OF THE FIGURES

FIG. 1 depicts the formulation of Nano-Folox. (A) A schematic representation of Nano-Folox formulated in microemulsions using a nanoprecipitation process. (B) The proposed mechanisms of Nano-Folox for synergistic chemo-immunotherapeutic effect. (C) The MALDI-TOF mass spectra and predicted chemical structure of the Pt(DACH).FnA precipitate (predicted exact mass: 780.23, observed m/z 780.96).

FIG. 2 depicts the physicochemical characterization of Nano-Folox. (A) TEM images of Pt(DACH).FnA and Nano-Folox (bar=100 nm). (B) Particle size (˜120 nm, polydispersity index=0.3) and zeta potential (˜5 mV) of Nano-Folox (also see the photograph). (C) The in vitro release of platinum from Nano-Folox in pH=5.5 and 7.4 (n=5).

FIG. 3 depicts in vitro studies of Nano-Folox. (A) Cellular uptake of platinum from OxP and Nano-Folox in CT26-FL3 cells was detected using ICP-MS (n=5, * p<0.05). (B) Cytotoxicity of CT26-FL3 cells treated with OxP and Nano-Folox was assessed using MTT assay (n=3, *p<0.05). (C) Apoptotic CT26-FL3 cells (%) treated with OxP and Nano-Folox was measured by Annexin V-FTIC and propidium iodide (PI) assay (n=3, * p<0.05, ** p<0.01). (D) The CRT exposure and HMGB1 release of CT26-FL3 cells treated with OxP and Nano-Folox were studied using immunofluorescence staining assay (n=3, *p<0.05). Results show that the CRT exposure and HMGB1 release were observed in ˜53% and ˜95% of CT26-FL3 cells following the treatment of Nano-Folox.

FIG. 4 depicts the pharmacokinetics and tissue distribution of Nano-Folox. (A) Plasma concentration of platinum (1 mg/kg) from OxP and Nano-Folox following a single i.v. injection through the mouse tail vein. The concentration of platinum was plotted using a semi-logarithmic scale (n=4). Pharmacokinetic parameters of platinum from OxP and Nano-Folox were shown in the table, in which t_{1/2}=half-life, AUC=area under the curve, CL=clearance, and Vd=volume of distribution (n=4, * p<0.05, **p<0.01). (B) Eight hours after a single i.v. injection the biodistribution of DiD-labeled Nano-Folox (1.5 mg/kg platinum) with/without AEAA targeting ligand was examined by the IVIS® Kinetics Optical System (n=4, * p<0.05). (C) The tissue distribution of platinum (1.5 mg/kg) from OxP and Nano-Folox with/without AEAA targeting ligand was also assessed using ICP-MS (n=4, *p<0.05).

FIG. 5 depicts anti-tumor effects of Nano-Folox in orthotopic CRC mice. (A) The treatment scheme. The IVIS images of orthotopic CT26-FL3 tumors on Day 32 following the treatment of PBS, Nano-Folox (1.5 mg/kg platinum), FOLFOX (3 mg/kg platinum, 90 mg/kg FnA and 50 mg/kg 5-Fu), and Nano-Folox/5-Fu. (B) The orthotopic CT26-FL3 tumor growth over a 35-day period following different treatments (n=6, * p<0.05, **p<0.01). (C) The survival of orthotopic CRC mice following different treatments (Median survival: PBS=41 days, Nano-Folox=49 days, FOLFOX=54 days, and Nano-Folox/5-Fu=68 days) (n=6, *** p<0.001).

FIG. 6 depicts chemo-immunotherapeutic mechanisms of Nano-Folox in orthotopic CRC mice. (A) Following the treatment scheme as shown in FIG. 5, TUNEL staining (green) of tumor tissues from animals treated with PBS, Nano-Folox (1.5 mg/kg platinum, i.v.), FOLFOX (3 mg/kg platinum, 90 mg/kg FnA and 50 mg/kg 5-Fu, i.p.) and Nano-Folox/5-Fu on Day 27 (The nuclei were stained using 4′,6-diamidino-2-phenylindole DAPI, blue). The Nano-Folox/5-Fu induced ˜7.2% apoptosis rate in the tumor, which is significantly higher than the other groups (n=4, * p<0.05, **p<0.01). (B) The immunofluorescence staining of tumors from animals treated with different groups using DAPI and anti-CD3 antibody (red) on Day 27. The T-cell infiltration rate (˜4.3%) was significantly improved by the Nano-Folox/5-Fu relative to the other groups (n=4, *p<0.05, **p<0.01). (C) The level of CD8⁺ cells, CD4⁺ cells, MHC DCs, CD86⁺ DCs, MDSCs, M2 cells, Treg cells, PD-L1 in tumors on Day 27, analyzed using flow cytometry (n=4, * p<0.05, **p<0.01). (D) The mRNA expression of CCL2, CXCL9, CXCL10, CXCL12, CXCL13, TNF-α and IFN-γ in tumors from animals treated with therapeutic groups relative to PBS, assessed using quantitative RT-PCR (n=4, *p<0.05, **p<0.01).

FIG. 7 depicts in vivo toxicity evaluation of Nano-Folox. (A) Following the treatment scheme as shown in FIG. 5, the animal body weight was recorded over a 35-day period (n=6). (B) On Day 27, major tissues (the heart, liver, spleen, lung and kidney) were collected and analyzed using the haematoxylin and eosin (H & E) staining assay, in order to determine histopathological changes. No significant histological changes were observed between PBS and therapeutic groups. (C) The blood and serum samples were collected on Day 27 and analyzed to determine the hematological toxicity and liver/kidney damage (n=4). No significant toxic signs were observed between PBS and therapeutic groups.

FIG. 8 depicts anti-tumor effects of Nano-Folox in mice with liver metastases. (A) The treatment scheme (red=Nano-Folox+5-Fu, purple=α-PD-L1 antibody). The IVIS images of CT26-FL3 liver metastases on Day 8, 12 and 16 following the treatment of PBS, anti-PD-L1 antibody (100 μg per animal, i.p.), Nano-Folox (1.5 mg/kg platinum, i.v.)+5-Fu (50 mg/kg, i.p.), and the combination. (B) The liver metastasis over a 16-day period following different treatments (n=5, * p<0.05). The ex vivo IVIS images of liver metastases on Day 16. (C) The survival of diseased animals following different treatments (Median survival: PBS=19 days, anti-PD-L1=21 days, Nano-Folox/5-Fu=34 days, and combination=48 days) (n=5, * p<0.05, ** p<0.01).

FIG. 9 depicts the preparation and physicochemical characterization of Nano-FdUMP. A schematic of Nano-FdUMP developed in microemulsions using nanoprecipitation technique (A). TEM image (bar=100 nm) (B). Size distribution (˜35 nm, polydispersity index 0.3) and surface charge (˜2 mV) of Nano-FdUMP (C). The in vitro release of fluorine drug from nanoprecipitates in Nano-FdUMP in pH=5.5 and 7.4 (n=4) (D). The stability of Nano-FdUMP following incubation of 10% serum-containing medium for 1, 2, 4 and 8 h at 37° C. (E).

FIG. 10 depicts in vitro studies of Nano-FdUMP. Cytotoxicity of CT26 and Hepa1-6 cells treated with 5-Fu and Nano-FdUMP (n=3, ** p<0.01) (A). Apoptotic CT26 and Hepa1-6 cells (%) treated with PBS, 5-Fu, Nano-dUMP and Nano-FdUMP were measured by Annexin V-FTIC and PI assay (n=3, *p<0.05, **p<0.01, relative to Nano-dUMP) (B). The ROS level in CT26 and Hepa1-6 cells treated with PBS, 5-Fu, Nano-dUMP and Nano-FdUMP (n=3, * p<0.05, ** p<0.01, relative to Nano-dUMP) (C). Apoptotic CT26 and Hepa1-6 cells (%) treated by Nano-FdUMP following incubation with or without NAC (n=3, * p<0.05, ** p<0.01, relative to PBS) (D).

FIG. 11 depicts synergistic ICD effects achieved by Nano-FdUMP and Nano-Folox. The exposure of CRT in CT26 and Hepa1-6 cells treated with PBS, Nano-FdUMP, Nano-Folox, and Nano-Folox/Nano-FdUMP following incubation with or without NAC (n=3, * p<0.05, ** p<0.01, *** p<0.001, relative to Nano-FdUMP) (A). The release of ATP from CT26 and Hepa1-6 cells into extracellular milieu treated with PBS, Nano-FdUMP, Nano-Folox, and Nano-Folox/Nano-FdUMP following incubation with or without NAC (n=3, * p<0.05, ** p<0.01, relative to PBS) (B). The secretion of HMBG1 in CT26 and Hepa1-6 cells treated with PBS, Nano-FdUMP, Nano-Folox, and Nano-Folox/Nano-FdUMP following incubation with or without NAC (n=3, * p<0.05, relative to Nano-FdUMP) (C).

FIG. 12 depicts blood circulation and biodistribution of Nano-FdUMP. 5-Fu and Nano-FdUMP were i.v. injected into orthotopic CRC and HCC mouse models. The concentration of fluorine drug on different time points was plotted (n=4). Half-life of 5-Fu and Nano-FdUMP was assessed using a one-compartmental model (A). Twelve hours post i.v. administration, the distribution of Did-labeled nanoformulations into tissues and tumors was detected (640 nm/670 nm) using IVIS® Kinetics Optical System (n=4, *p<0.05) in mice grafted with CRC (B) and HCC (C). In HCC model, AEAA-targeted nanoformulation was specifically accumulated inside liver tumor, which was confirmed by colocalization of NPs (fluorescent imaging from DiD dye) and tumor tissue (bioluminescence imaging from visible light produced by the interaction between luciferase and luciferin).

FIG. 13 depicts hemo-immunotherapeutic effects of two nanoformulations in orthotopic CRC mouse model. Treatment schedule and IVIS images (A). The CT26-FL3 tumor growth over a 35-day period (n=6, * p<0.05, ** p<0.01) (B). Animal survival (median survival: PBS=40 days, Nano-FdUMP=45 days, Nano-FdUMP with OxP and FnA=49 days, and Nano-Folox with 5-Fu=56 days) (n=6, **p<0.01) (C). Immunofluorescent staining of tumors on Day 24 (DNA fragments=green; nuclei=blue) to determine apoptosis (n=4, * p<0.05, relative to Nano-Folox/5-Fu) (D). Immunofluorescence staining of tumors on Day 24 (CD3=red; nuclei=blue) to determine T cell infiltration (n=4, **p<0.01, relative to Nano-Folox/5-Fu) (E). Level of CD8⁺ T cells, CD4⁺ T cells, memory CD8⁺ T cells, memory CD4⁺ T cells, activated DCs, MDSCs, Tregs and M2 cells in tumors on Day 24, analyzed by flow cytometry (n=4, * p<0.05, ** p<0.01, relative to Nano-Folox/5-Fu) (F). The mRNA expression of IFN-γ, TNF-α, IL-12, IL-4, IL-6 and IL-10 in tumors on Day 24 (n=4, * p<0.05, relative to Nano-Folox/5-Fu) (G). Orthotopic CT26-FL3 tumor growth treated with Nano-FdUMP/Nano-Folox after removal of CD4⁺ or CD8⁺ T cells (n=4, * p<0.05, **p<0.01) (H).

FIG. 14 depicts chemo-immunotherapeutic effects of two nanoformulations in orthotopic HCC mouse model. Treatment schedule and IVIS images (A). The Hepa1-6-Luc tumor growth over a 32-day period (n=6, * p<0.05, ** p<0.01) (B). Animal survival (median survival: PBS=36 days, Nano-FdUMP=43 days, Nano-FdUMP with OxP and FnA=47 days, and Nano-Folox with 5-Fu=53 days) (n=6, *** p<0.001) (C). Immunofluorescent staining of tumors on Day 23 (DNA fragments=green; nuclei=blue) to determine apoptosis (n=4, * p<0.05, relative to Nano-Folox/5-Fu) (D). Immunofluorescence staining of tumors on Day 23 (CD3=red; nuclei=blue) to determine T cell infiltration (n=4, **p<0.01, relative to Nano-Folox/5-Fu) (E). Level of CD8⁺ T cells, CD4⁺ T cells, memory CD8⁺ T cells, memory CD4⁺ T cells, activated DCs, MDSCs, Tregs and M2 cells in tumors on Day 23, analyzed by flow cytometry (n=4, * p<0.05, relative to Nano-Folox/5-Fu) (F). The mRNA expression of IFN-γ, TNF-α, IL-12, IL-4, IL-6 and IL-10 in tumors on Day 23 (n=4, * p<0.05, relative to Nano-Folox/5-Fu) (G). Orthotopic Hepa1-6-Luc tumor growth treated with Nano-FdUMP/Nano-Folox after removal of CD4⁺ or CD8⁺ T cells (n=4, * p<0.05, ** p<0.01) (H).

FIG. 15 depicts the combination therapy of Nano-FdUMP/Nano-Folox and anti-PD-L1 antibody for CRC liver metastasis mouse model. Treatment schedule and IVIS images (A). The liver metastases over a 16-day period (n=6, * p<0.05, ** p<0.01) (B). Animal survival (median survival: PBS=20 days, anti-PD-L1 antibody=21 days, and Nano-FdUMP/Nano-Folox=48 days) (n=6, *** p<0.01) (C). Immunofluorescent staining of tumors on Day 12 (DNA fragments=green; nuclei=blue) to determine apoptosis (n=4, **p<0.01, relative to Nano-FdUMP/Nano-Folox) (D). Immunofluorescence staining of tumors on Day 12 (CD3=red; nuclei=blue) to determine T cell infiltration (n=4, ** p<0.01, relative to Nano-FdUMP/Nano-Folox) (E). Level of CD8⁺ T cells, CD4⁺ T cells, memory CD8⁺ T cells, memory CD4⁺ T cells, and activated DCs in tumors on Day 12, analyzed by flow cytometry (n=4, *p<0.05, ** p<0.01, relative to anti-PD-L1 antibody) (F). The mRNA expression of IFN-γ, IL-12, IL-4, IL-6 and IL-10 in tumors on Day 12 (n=4, * p<0.05, ** p<0.01, relative to anti-PD-L1 antibody) (G).

FIG. 16 depicts the physicochemical characterization of non-targeted Nano-FdUMP. TEM image (bar=100 nm) (A). Size distribution (˜38 nm, polydispersity index 0.3) and surface charge (˜5 mV) (B). The in vitro release of fluorine drug from nanoprecipitates in pH=5.5 and 7.4 (n=4) (C). No significant aggregation (from ˜35 to 50 nm) was caused in 10% serum-containing medium up to 8 h at 37° C. (D).

FIG. 17 depicts the blood circulation of non-targeted Nano-FdUMP in orthotopic CRC and HCC mouse models. Following i.v. injection, the concentration of fluorine drug on different time points was plotted (n=4). Results showed that non-targeted Nano-FdUMP demonstrated similar blood circulation recorded by targeted counterpart.

FIG. 18 depicts the toxicity of Nano-FdUMP in healthy BALB/C mice. The body weight over a 35-day period following treatment of PBS and Nano-FdUMP containing 5, 10, 25 and 50 mg/kg FdUMP on Day 1, 3 and 5. (A). The overall condition of animals (n=5) based on body condition scoring [BCS, IACUC Guidelines along with other criteria (e.g. hunched posture, ruffled hair coat, and reluctance to move)] (B). At the endpoint, the number of animals compliant with BCS index was presented. Results of non-targeted Nano-FdUMP were similar to those observed in targeted counterpart (Data not shown).

FIG. 19 depicts the therapeutic efficacy of Nano-FdUMP in orthotopic CRC and HCC mouse models. Following treatment schedule as described in FIGS. 13 and 14, Nano-FdUMP at doses of 10 and 25 mg/kg FdUMP achieved significantly improved antitumor efficacy as compared to PBS (n=5, * p<0.05).

FIG. 20 depicts therapeutic efficacy of Nano-FdUMP with/without AEAA at dose of 10 mg/kg FdUMP in orthotopic CRC and HCC mouse models. Following treatment schedule as described in FIGS. 13 and 14, non-targeted Nano-FdUMP could not slow down tumor growth as compared to PBS, but AEAA-targeted Nano-FdUMP achieved significantly improved antitumor efficacy than PBS and non-targeted Nano-FdUMP (n=5, * p<0.05).

FIG. 21 depicts therapeutic efficacy of Nano-FdUMP with/without AEAA at dose of 10 mg/kg FdUMP in orthotopic CRC and HCC mouse models. Following treatment schedule as described in FIGS. 13 and 14, non-targeted Nano-FdUMP could not slow down tumor growth as compared to PBS, but AEAA-targeted Nano-FdUMP achieved significantly improved antitumor efficacy than PBS and non-targeted Nano-FdUMP (n=5, * p<0.05).

FIG. 22 depicts the toxicity studies of combination of two nanoformulations in healthy BALB/C (A) and C57BL/6 (B) mice. The body weight over a 35-day period following treatment of PBS and combination of two nanoformulations (Nano-Folox containing 1.5 mg/kg platinum drug was i.v. injected into mice on Day 1, 3 and 5. Eight hours post injection, Nano-FdUMP containing 10 mg/kg fluorine drug was i.v. injected into mice). Results show that no significant change was found in body weight and hematological/liver/kidney functions following treatment of two nanoformulations as compared to PBS (n=5).

FIG. 23 depicts a (A) a schematic of nano-FOLOX formulated in microemulsions using the nanoprecipitation process. (B) a schematic of nano-FdUMP formulated in microemulsions using the nanoprecipitation process.

FIG. 24 depicts (A) a polymer-encapsulated particle comprising stabilized single-lipid layer nano-FOLOX cores, and stabilized single-lipid layer nano-FdUMP cores. (B) a polymer-encapsulated particle comprising SN-38, stabilized single-lipid layer nano-FOLOX cores, and stabilized single-lipid layer nano-FdUMP cores.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Provided herein are methods and compositions for the delivery of nano-folox for the treatment of cancer. Compositions include delivery system complexes comprising a combination of folinic acid (FnA) and 5-fluorouracil (5-Fu) and oxaliplatin (OxP). In another embodiments, compositions include delivery system complexes comprising a combination of folinic acid (FnA) or 5-Fluoro-2′-deoxyuridine 5′-monophosphate (FdUMP) or combinations thereof. Methods include administering the compositions along with an anti-PDL1 antibody.

It is known that cancer cells can show diversity of genetic, transcriptomic, epigenetic and phenotypic profiles within/between tumors and metastases during the course of disease⁵⁰. As a result of this heterogeneity, conventional monotherapeutic approaches often fail to provide a safe and effective treatment for patients. Therefore, the amalgamation of therapeutic agents, which mediate multiple anticancer pathways, may achieve a synergistic outcome^{51 52 53 54}. Indeed, the combination of chemotherapy and immunotherapy holds great promise for eliciting better anticancer results than either of monotherapies⁵⁵. Recently, the development of non-viral nano-delivery technologies have demonstrated the possibility to simultaneously formulate chemotherapeutic and immunotherapeutic agents, achieving chemo-immunotherapeutic responses^{56 57} As described herein, a nanoprecipitation technique was employed to develop an AEAA-targeted lipid-based NP for co-formulating OxP derivative and FnA, with the aim of facilitating chemo-immunotherapy for CRC.

The resultant formulation, namely Nano-Folox, demonstrated favorable physicochemical profiles, in terms of particle size, surface charge, and drug release. Following i.v. administration prolonged systemic exposure and enhanced tumor accumulation of platinum were achieved by Nano-Folox. When a combination of Nano-Folox and 5-Fu was given to orthotopic CRC mice, the anti-tumor efficacy was significantly higher than the FOLFOX at a higher dose (2-fold platinum).

It has been reported that differentiation of naïve T cells is highly associated with antigen availability to DCs,⁷⁶ and higher amount and longer duration of antigen stimulation produce larger number of effector and memory T ICD can induce exposure of damage-associated molecular patterns (DAMPs) from dying or dead cancer cells, resulting in antigen presentation to DCs for tumor-specific T cell response.⁷⁸ It has been also reported that the induction of ICD is accompanied with the formation of reactive oxygen species (ROS),⁷⁸ and the ICD efficacy may be enhanced by ROS-inducing strategies.⁷⁹⁻⁸¹ Therefore, we hypothesize that the ROS induction may be safely and effectively achieved by targeted delivery of 5-Fu using nano delivery systems, which will synergize with Nano-Folox to induce effector and memory T cells for tumor-specific killing and protective response. Therefore, an AEAA-targeted PEGylated lipid NP (termed Nano-FdUMP) was produced using nanoprecipitation technique for delivery of 5-Fluoro-2′-deoxyuridine 5′-monophosphate (FdUMP, an active 5-Fu metabolite)⁸².

Also advantageously, the anti-PD-L1 antibody synergized with the Nano-Folox/5-Fu resulting in a retardation of liver metastasis in mice. The anticancer mechanisms of this combination strategy are likely due to 1) the synergistic apoptotic effects is achieved by OxP-based DNA-adduct formation and by FnA-sensitized 5-Fu-mediated DNA damage; 2) the OxP derivative as the ICD inducer dramatically remodels the tumor immune microenvironment resulting in effective immunotherapy, particularly when combined with 5-Fu; 3) the application of anti-PD-L1 antibody blocks the PD-L1/PD-1 inhibitory signaling, which enhance the immune response of T lymphocytes achieved by Nano-Folox/5-Fu.

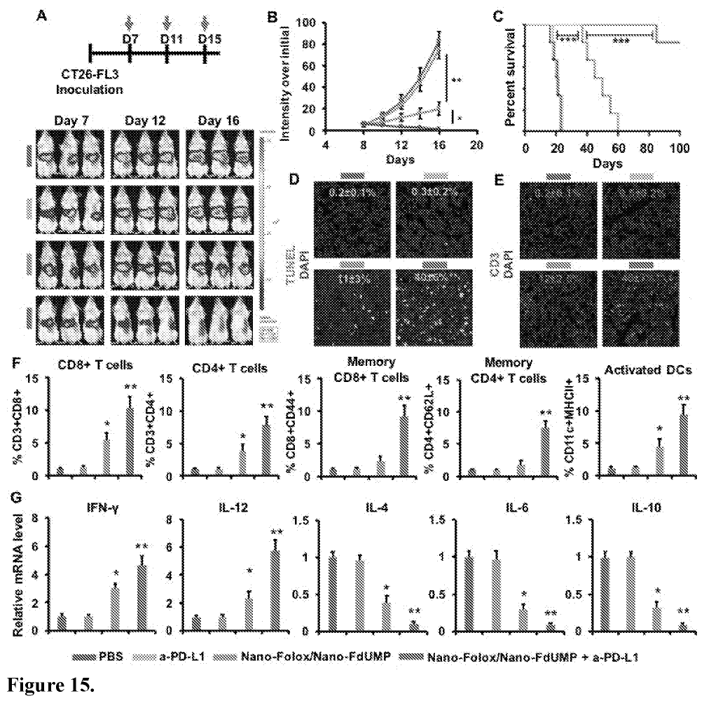

Moreover, immune checkpoint inhibitors (e.g. anti-PD-L1 mAb) have demonstrated efficacy in different cancers, but response rate is still poor in CRC patients. Only a minor population of patients, who are diagnosed with microsatellite instable (MSI) CRC (˜15% of total population),¹¹⁴ respond to anti-PD-L1 mAb as a monotherapy.¹¹⁵ It is now known that the lack of T cell infiltration in tumor (also characterized as “cold” tumor) causes inefficiency of immune checkpoint inhibitors.¹¹⁶ The shift of “cold” tumor to “hot” one potentially enhances efficacy of checkpoint blockade.¹¹⁷ The combination of Nano-FdUMP and Nano-Folox was able to induce ICD-associated antitumor immunity, which significantly reprogrammed immunosuppressive TME, improving antitumor efficacy against CRC liver metastasis (established by CT26-FL3 cells, an MSS CRC cell line)^{118,119} in combination with anti-PD-L1 mAb (FIG. 15). The combination of Nano-Folox/Nano-FdUMP and anti-PD-L1 antibody significantly inhibited CRC liver metastasis, induced tumor-specific memory response, and led to long-term survival in mice. Therefore, the “Nano-FdUMP/Nano-Folox+anti-PD-L1 mAb” strategy will potentially achieve a superior outcome for CRC patients (particularly for microsatellite stable (MSS) ones, up to 85% of total population) at primary and metastatic stages.

Colorectal cancer (CRC) is associated with high morbidity and mortality, with an estimated burden increase to over 2.2 million new cases and 1.1 million fatalities by 2030 globally¹. Surgical resection provides the potential cure for patients with CRC in early stage, and chemotherapy is the mainstay of treatment for advanced and metastatic CRC². The combination of folinic acid (FnA, also known as leucovorin), 5-fluorouracil (5-Fu) and oxaliplatin (OxP) commonly known as FOLFOX³, has been applied for patients with CRC at stage II/III⁴ and when liver metastases occur⁵. Although FOLFOX has improved the survival, improvements to the therapeutic modality is needed because dose-limiting side effects, high expenses, and long course of treatment still limit the clinical application³. Therefore, new FOLFOX strategies, in terms of improving the therapeutic efficacy while reducing toxicity, cost and inconvenience (i.e. time-consuming treatment scheme), are needed.

5-Fu, an antimetabolite chemotherapeutic drug, has been widely used in the treatment of CRC for decades. The therapeutic efficacy of 5-Fu is resulted from the intercalation of fluoronucleotides into RNA/DNA and from the inactivation of thymidylate synthase (TS, the nucleotide synthetic enzyme)¹⁶. In addition, the anticancer effect of 5-Fu can be improved by FnA through enhancing TS inhibition^{17 18}.

It has been reported that differentiation of naïve T cells is highly associated with antigen availability to DCs,⁷⁶ and higher amount and longer duration of antigen stimulation produce larger number of effector and memory T cells.⁷⁷ ICD can induce exposure of damage-associated molecular patterns (DAMPs) from dying or dead cancer cells, resulting in antigen presentation to DCs for tumor-specific T cell response.⁷⁸ It has been also reported that the induction of ICD is accompanied with the formation of reactive oxygen species (ROS),⁷⁸ and the ICD efficacy may be enhanced by ROS-inducing strategies.⁷⁹⁻⁸¹ Therefore, we hypothesize that the ROS induction may be safely and effectively achieved by targeted delivery of 5-Fu using nano delivery systems, which will synergize with Nano-Folox to induce effector and memory T cells for tumor-specific killing and protective response. Therefore, an AEAA-targeted PEGylated lipid NP (termed Nano-FdUMP) was produced using nanoprecipitation technique for delivery of 5-Fluoro-2′-deoxyuridine 5′-monophosphate (FdUMP, an active 5-Fu metabolite)⁸². In clinic trials, OxP has presented additive or synergistic activity when combined with 5-Fu and FnA¹⁹. However, the clinical application of this combination strategy (FOLFOX) is still retarded by several issues such as toxic effects, cost increase and inconvenience. In this study, a NP-based FOLFOX strategy was developed with an aim of significantly improving therapeutic efficacy and efficiently overcoming the limitations.

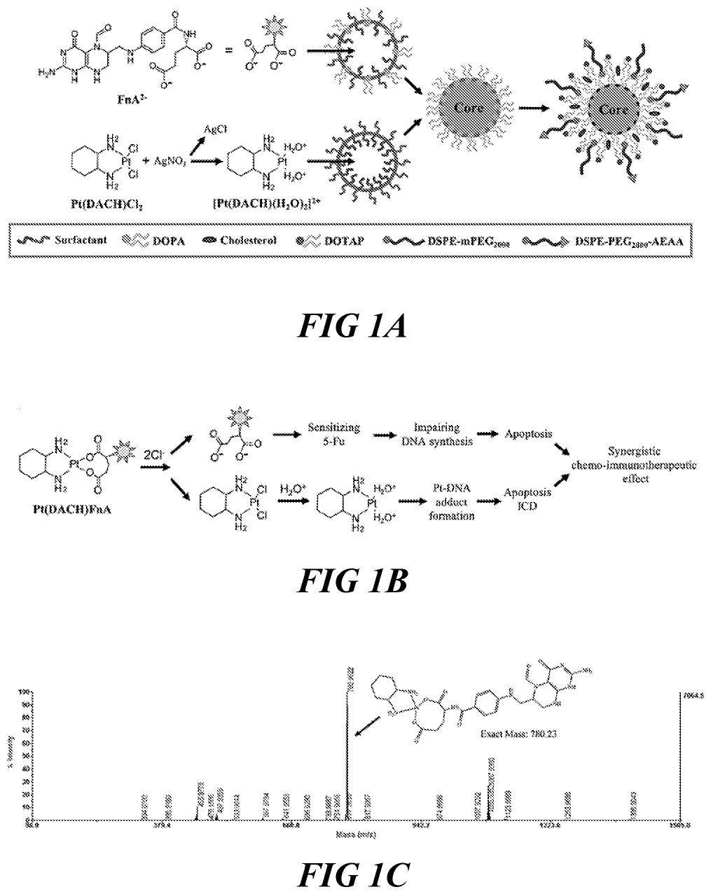

Microemulsion lipid-based cisplatin nanoparticles (NP) are known^{6 7 8}. As described herein, a precipitate was produced by a reaction between dihydrate(1,2-diaminocyclohexane)platinum(II) ([Pt(DACH)(H₂O)₂]²⁺, the active form of OxP) and FnA, which was stabilized by 1,2-dioleoyl-sn-glycero-3-phosphate (DOPA). The stabilized precipitate was formulated into a NP composed of 1,2-dioleoyl-3-trimethylammonium-propane (DOTAP), cholesterol, and 1,2-distearoyl-sn-glycero-3-phosphoethanolamine-polyethylene glycol-2000 conjugated with aminoethyl anisamide (DSPE-PEG-AEAA) (FIG. 1). The resulting formulation (namely Nano-Folox) was investigated in combination with 5-Fu and anti-PD-L1 mAb for synergistic chemo-immunotherapeutic efficacy in mice with orthotopic CRC and liver metastasis.

In summary, the combination strategy described herein provides a potential FOLFOX modality with reduced cycle and less dosage, in a hope of achieving a superior chemo-immunotherapeutic response for patients with primary and metastatic CRC.

### I. Compositions

In certain embodiments, the subject matter described herein is directed to a compound having the structure:

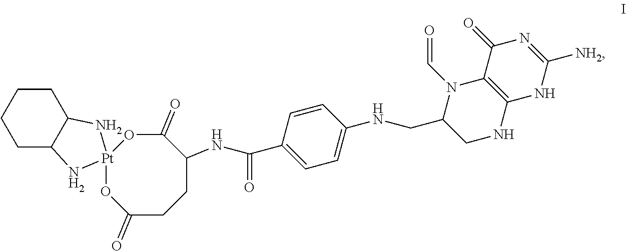

this structure is also referred to herein as an OxP-FnA complex, as a complex of dihydrate(1,2-diaminocyclohexane)platinum(II)-folinic acid, as the precipitate or as Folox. Scheme 1 depicts a general synthetic route to the compound of Formula I.

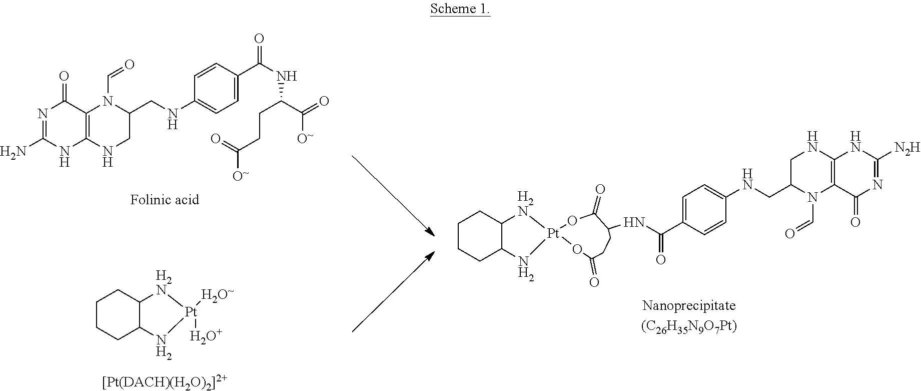

The compound of Formula I can be in the form of a nanoprecipitate (C₂₆H₃₅N₉O₇PO prepared by condensing folinic acid and Pt(DACH)(H₂O)₂]²⁺ as described elsewhere herein. The dihydrate(1,2-diaminocyclohexane)platinum(II) ([Pt(DACH)(H₂O)₂]²⁺, the active form of oxaliplatin) was reacted with folinic acid to form a nanoprecipitate (C₂₆H₃₅N₉O₇Pt, see also, FIG. 1). In embodiments, the nanoprecipitate can be coated with a coating, having one or more layers, wherein one of the layers comprise 1,2-dioleoyl-3-trimethylammonium-propane (DOTAP), cholesterol, 1,2-distearoyl-sn-glycero-3-phosphoethanolamine-polyethylene glycol-2000 (DSPE-PEG) and DSPE-PEG conjugated with aminoethyl anisamide (DSPE-PEG-AEAA).

In certain embodiments, the subject matter described herein is directed to delivery system complexes comprising the compound of Formula I. As used herein, a “delivery system complex” or “delivery system” refer to a complex comprising a compound of Formula I and a means for delivering the bioactive compound of Formula I to a cell, physiological site, or tissue.

In certain embodiments, the subject matter described herein is directed to delivery system complexes comprising the compound of Formula I, wherein the delivery system complex comprises a liposome-encapsulated compound of Formula I.

In certain embodiments, the subject matter described herein is directed to delivery system complexes comprising the compound of Formula I, wherein the delivery system complex comprises a liposome-encapsulated compound of Formula I, wherein the liposome comprises a lipid bilayer. In certain embodiments, the delivery system comprises an asymmetric bilayer.

In certain embodiments, the subject matter disclosed herein is directed to a delivery system complex comprising a core, wherein the core comprises a complex of dihydrate(1,2-diaminocyclohexane)platinum(II)-folinic acid, wherein said core is encapsulated by a liposome. A useful complex is dihydrate(1,2-diaminocyclohexane)platinum(II)-folinic acid has the following structure:

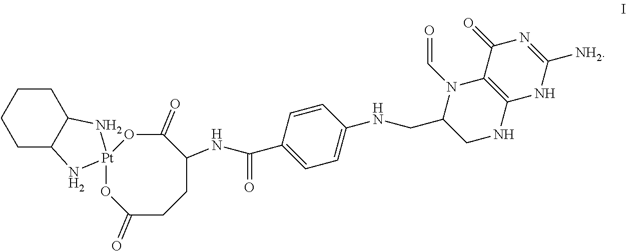

In certain embodiments, the subject matter described herein is directed to a delivery system complex comprising, a core comprising an anti-metabolite complex, said anti-metabolite complex comprising a 5-fluorouracil active metabolite, wherein said core is encapsulated by a liposome. In certain embodiments, the complex is a precipitate. In certain embodiments, the delivery system comprises an asymmetric bilayer.

In certain embodiments, the subject matter disclosed herein is directed to a delivery system complex comprising a core, wherein the core comprises a CaP precipitate made from CaCl₂ and (NH₄)₂HPO₄ and 5-fluorouracil active metabolite, wherein said core is encapsulated by a liposome. In one embodiment, the 5-fluorouracil active metabolite is 5-Fluoro-2′-deoxyuridine 5′-monophosphate. In certain embodiments, the delivery system comprises an asymmetric bilayer.

In certain embodiments, the subject matter described herein is directed to a delivery system complex comprising, a first type of stabilized single-lipid layer core comprising an anti-metabolite complex, a second type of stabilized single-lipid layer core comprising the compound of Formula I, wherein the cores are encapsulated by a polymer, such as PLGA, PLGA-PEG or PLGA-PEG-AEAA. In certain embodiments, the single-lipid is the phospholipid, DOPA.

In certain embodiments, the subject matter described herein is directed to a delivery system complex comprising, a first type of stabilized single-lipid layer core comprising an anti-metabolite complex, a second type of stabilized single-lipid layer core comprising the compound of Formula I, and irinotecan (SN-38), wherein the cores and SN-38 are encapsulated by a polymer, such as PLGA, PLGA-PEG or PLGA-PEG-AEAA. In certain embodiments, the single-lipid is the phospholipid, DOPA.

In certain embodiments, the liposome of the delivery system complex comprises a lipid bilayer having an inner leaflet and an outer leaflet.

In certain embodiments, the “anti-metabolite complex” as used herein refers to a CaP precipitate made from CaCl₂ and (NH₄)₂HPO₄ and 5-fluorouracil active metabolite.

In certain embodiments, the outer leaflet comprises a lipid-polyethylene glycol (lipid-PEG) conjugate. In certain embodiments, the lipid-PEG conjugate comprises PEG in an amount between about 5 mol % to about 50 mol % of total surface lipid. In certain embodiments, the lipid-PEG conjugate comprises a PEG molecule having a molecular weight of about 2000 g/mol. In certain embodiments, the lipid-PEG conjugate comprises a 1,2-distearoyl-sn-glycero-3-phosphoethanolamine-N-carboxy-polyethylene glycol₂₀₀₀ (DSPE-PEG₂₀₀₀).

In certain embodiments, the outer leaflet comprises a targeting ligand, thereby forming a targeted delivery system complex, wherein said targeting ligand targets said targeted delivery system complex to a targeted cell. In certain embodiments, the targeting ligand is DSPE-PEG conjugated with aminoethyl anisamide (DSPE-PEG-AEAA). This targeting ligand is shown herein to co-deliver oxaliplatin and folinic acid.

The delivery system complexes described herein can contain many cores. As described herein, the complexes that contain one or more types of cores can contain any number of cores of each type.

In certain embodiments, the delivery system complex has a diameter of about 50 nm to about 900 nm. In certain embodiments, the delivery system complex has an average diameter of about 120 nm.

In certain embodiments, the outer leaflet of the delivery system complex comprises a cationic lipid. In certain embodiments, the cationic lipid is DOTAP.

In certain embodiments, the inner leaflet of the delivery system complex comprises an amphiphilic lipid. In certain embodiments, the amphiphilic lipid is DOPA.

The presently disclosed delivery system complexes can comprise a liposome that encapsulates an OxP-FnA complex. Liposomes are self-assembling, substantially spherical vesicles comprising a lipid bilayer that encircles a core, which can be aqueous, wherein the lipid bilayer comprises amphipathic lipids having hydrophilic headgroups and hydrophobic tails, in which the hydrophilic headgroups of the amphipathic lipid molecules are oriented toward the core or surrounding solution, while the hydrophobic tails orient toward the interior of the bilayer. The lipid bilayer structure thereby comprises two opposing monolayers that are referred to as the “inner leaflet” and the “outer leaflet,” wherein the hydrophobic tails are shielded from contact with the surrounding medium. The “inner leaflet” is the monolayer wherein the hydrophilic head groups are oriented toward the core of the liposome. The “outer leaflet” is the monolayer comprising amphipathic lipids, wherein the hydrophilic head groups are oriented towards the outer surface of the liposome. Liposomes typically have a diameter ranging from about 25 nm to about 1 μm. (see, e.g., Shah (ed.) (1998) Micelles, Microemulsions, and Monolayers: Science and Technology, Marcel Dekker; Janoff (ed.) (1998) Liposomes: Rational Design, Marcel Dekker). The term “liposome” encompasses both multilamellar liposomes comprised of anywhere from two to hundreds of concentric lipid bilayers alternating with layers of an aqueous phase and unilamellar vesicles that are comprised of a single lipid bilayer. Methods for making liposomes are well known in the art and are described elsewhere herein.

As used herein, the term “lipid” refers to a member of a group of organic compounds that has lipophilic or amphipathic properties, including, but not limited to, fats, fatty oils, essential oils, waxes, steroids, sterols, phospholipids, glycolipids, sulpholipids, aminolipids, chromolipids (lipochromes), and fatty acids. The term “lipid” encompasses both naturally occurring and synthetically produced lipids. “Lipophilic” refers to those organic compounds that dissolve in fats, oils, lipids, and non-polar solvents, such as organic solvents. Lipophilic compounds are sparingly soluble or insoluble in water. Thus, lipophilic compounds are hydrophobic. Amphipathic lipids, also referred to herein as “amphiphilic lipids” refer to a lipid molecule having both hydrophilic and hydrophobic characteristics. The hydrophobic group of an amphipathic lipid, as described in more detail immediately herein below, can be a long chain hydrocarbon group. The hydrophilic group of an amphipathic lipid can include a charged group, e.g., an anionic or a cationic group, or a polar, uncharged group. Amphipathic lipids can have multiple hydrophobic groups, multiple hydrophilic groups, and combinations thereof. Because of the presence of both a hydrophobic group and a hydrophilic group, amphipathic lipids can be soluble in water, and to some extent, in organic solvents.

As used herein, “hydrophilic” is a physical property of a molecule that is capable of hydrogen bonding with a water (H₂O) molecule and is soluble in water and other polar solvents. The terms “hydrophilic” and “polar” can be used interchangeably. Hydrophilic characteristics derive from the presence of polar or charged groups, such as carbohydrates, phosphate, carboxylic, sulfato, amino, sulfhydryl, nitro, hydroxy and other like groups.

Conversely, the term “hydrophobic” is a physical property of a molecule that is repelled from a mass of water and can be referred to as “nonpolar,” or “apolar,” all of which are terms that can be used interchangeably with “hydrophobic.” Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, dioleoyl phosphatidic acid, and dilinoleoylphosphatidylcholine. Other compounds lacking in phosphorus, such as sphingolipid, glycosphingolipid families, diacylglycerols and β-acyloxyacids, also are within the group designated as amphipathic lipids.

In some embodiments, the liposome or lipid bilayer comprises cationic lipids. As used herein, the term “cationic lipid” encompasses any of a number of lipid species that carry a net positive charge at physiological pH, which can be determined using any method known to one of skill in the art. Such lipids include, but are not limited to, the cationic lipids of formula (I) disclosed in International Application No. PCT/US2009/042476, entitled “Methods and Compositions Comprising Novel Cationic Lipids,” which was filed on May 1, 2009, and is herein incorporated by reference in its entirety. These include, but are not limited to, N-methyl-N-(2-(arginoylamino) ethyl)-N, N-Di octadecyl aminium chloride or di stearoyl arginyl ammonium chloride] (DSAA), N,N-di-myristoyl-N-methyl-N-2[N′—(N⁶-guanidino-L-lysinyl)] aminoethyl ammonium chloride (DMGLA), N,N-dimyristoyl-N-methyl-N-2[N²-guanidino-L-lysinyl] aminoethyl ammonium chloride, N,N-dimyristoyl-N-methyl-N-2[N′—(N2,N6-di-guanidino-L-lysinyl)] aminoethyl ammonium chloride, and N,N-di-stearoyl-N-methyl-N-2[N′—(N6-guanidino-L-lysinyl)] aminoethyl ammonium chloride (DSGLA). Other non-limiting examples of cationic lipids that can be present in the liposome or lipid bilayer of the presently disclosed delivery system complexes include N,N-dioleyl-N,N-dimethylammonium chloride (“DODAC”); N-(2,3-dioleoyloxy) propyl)-N,N,N-trimethylammonium chloride (“DOTAP”); N-(2,3-dioleyloxy) propyl)-N,N,N-trimethylammonium chloride (“DOTMA”) or other N—(N,N-1-dialkoxy)-alkyl-N,N,N-trisubstituted ammonium surfactants; N,N-distearyl-N,N-dimethylammonium bromide (“DDAB”); 3-(N—(N′,N′-dimethylaminoethane)-carbamoyl) cholesterol (“DC-Chol”) and N-(1,2-dimyristyloxyprop-3-yl)-N,N-dimethyl-N-hydroxyethyl ammonium bromide (“DMRIE”); 1,3-dioleoyl-3-trimethylammonium-propane, N-(1-(2,3-dioleyloxy)propyl)-N-(2-(sperminecarboxamido)ethyl)-N,N-dimethy-1 ammonium trifluoro-acetate (DOSPA); GAP-DLRIE; DMDHP; 3-β[⁴N-(¹N,⁸N-diguanidino spermidine)-carbamoyl] cholesterol (BGSC); 3-β[N,N-diguanidinoethyl-aminoethane)-carbamoyl] cholesterol (BGTC); N,N¹,N²,N³ Tetra-methyltetrapalmityl spermine (cellfectin); N-t-butyl-N′-tetradecyl-3-tetradecyl-aminopropion-amidine (CLONfectin); dimethyldioctadecyl ammonium bromide (DDAB); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3 dioleoyloxy-1,4-butanediammonium iodide) (Tfx-50); 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DL-1,2-dioleoyl-3-dimethylaminopropyl-β-hydroxyethylammonium (DORI) or DL-1,2-O-dioleoyl-3-dimethylaminopropyl-β-hydroxyethylammonium (DORIE) or analogs thereof as disclosed in International Application Publication No. WO 93/03709, which is herein incorporated by reference in its entirety; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 5,283,185, which is herein incorporated by reference in its entirety; cholesteryl-3β-carboxyl-amido-ethylenetrimethylammonium iodide; 1-dimethylamino-3-trimethylammonio-DL-2-propyl-cholesteryl carboxylate iodide; cholesteryl-3-β-carboxyamidoethyleneamine; cholesteryl-3-β-oxysuccinamido-ethylenetrimethylammonium iodide; 1-dimethylamino-3-trimethylammonio-DL-2-propyl-cholesteryl-3-β-oxysuccinate iodide; 2-(2-trimethylammonio)-ethylmethylamino ethyl-cholesteryl-3-β-oxysuccinate iodide; and 3-β-N-(polyethyleneimine)-carbamoylcholesterol.

In some embodiments, the liposomes or lipid bilayers can contain co-lipids that are negatively charged or neutral. As used herein, a “co-lipid” refers to a non-cationic lipid, which includes neutral (uncharged) or anionic lipids. The term “neutral lipid” refers to any of a number of lipid species that exist either in an uncharged or neutral zwitterionic form at physiological pH. The term “anionic lipid” encompasses any of a number of lipid species that carry a net negative charge at physiological pH. Co-lipids can include, but are not limited to, diacylphosphatidylcholine, diacylphosphatidylethanolamine, ceramide, sphingomyelin, cephalin, cholesterol, cerebrosides and diacylglycerols, phospholipid-related materials, such as lecithin, phosphatidylethanolamine, lysolecithin, lysophosphatidylethanolamine, phosphatidylserine, phosphatidylinositol, cardiolipin, phosphatidic acid, dicetylphosphate, di stearoylphosphatidylcholine (DSPC), dioleoylphosphatidylcholine (DOPC), dipalmitoylphosphatidylcholine (DPPC), dioleoylphosphatidylglycerol (DOPG), palmitoyloleyolphosphatidylglycerol (POPG), dipalmitoylphosphatidylglycerol (DPPG), dioleoyl-phosphatidylethanolamine (DOPE), palmitoyloleoylphosphatidylchol-ine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dioleoyl phosphatidic acid (DOPA), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, lysophosphatidylcholine, and dioctadecyldimethyl ammonium bromide and the like. Co-lipids also include polyethylene glycol-based polymers such as PEG 2000, PEG 5000 and polyethylene glycol conjugated to phospholipids or to ceramides, as described in U.S. Pat. No. 5,820,873, herein incorporated by reference in its entirety.

In some embodiments, the liposome of the delivery system complex is a cationic liposome and in other embodiments, the liposome is anionic. The term “cationic liposome” as used herein is intended to encompass any liposome as defined above which has a net positive charge or has a zeta potential of greater than 0 mV at physiological pH. Alternatively, the term “anionic liposome” refers to a liposome as defined above which has a net negative charge or a zeta potential of less than 0 mV at physiological pH. The zeta potential or charge of the liposome can be measured using any method known to one of skill in the art. It should be noted that the liposome itself is the entity that is being determined as cationic or anionic, meaning that the liposome that has a measurable positive charge or negative charge at physiological pH, respectively, can, within an in vivo environment, become attached to other substances or may be associated with other charged components within the aqueous core of the liposome, which can thereby result in the formation of a structure that does not have a net charge. After a delivery system complex comprising a cationic or anionic liposome is produced, molecules such as lipid-PEG conjugates can be post-inserted into the bilayer of the liposome as described elsewhere herein, thus shielding the surface charge of the delivery system complex.

In those embodiments in which the liposome of the delivery system complex is a cationic liposome, the cationic liposome need not be comprised completely of cationic lipids, however, but must be comprised of a sufficient amount of cationic lipids such that the liposome has a positive charge at physiological pH. The cationic liposomes also can contain co-lipids that are negatively charged or neutral, so long as the net charge of the liposome is positive and/or the surface of the liposome is positively charged at physiological pH. In these embodiments, the ratio of cationic lipids to co-lipids is such that the overall charge of the resulting liposome is positive at physiological pH. For example, cationic lipids are present in the cationic liposome at from about 10 mole % to about 100 mole % of total liposomal lipid, in some embodiments, from about 20 mole % to about 80 mole % and, in other embodiments, from about 20 mole % to about 60 mole %. Neutral lipids, when included in the cationic liposome, can be present at a concentration of from about 0 mole % to about 90 mole % of the total liposomal lipid, in some embodiments from about 20 mole % to about 80 mole %, and in other embodiments, from about 40 mole % to about 80 mole %. Anionic lipids, when included in the cationic liposome, can be present at a concentration ranging from about 0 mole % to about 49 mole % of the total liposomal lipid, and in certain embodiments, from about 0 mole % to about 40 mole %.

In some embodiments, the cationic liposome of the delivery system complex comprises a cationic lipid and the neutral co-lipid cholesterol at a 1:1 molar ratio. In some of these embodiments, the cationic lipid comprises DOTAP.

Likewise, in those embodiments in which the liposome of the delivery system complex is an anionic liposome, the anionic liposome need not be comprised completely of anionic lipids, however, but must be comprised of a sufficient amount of anionic lipids such that the liposome has a negative charge at physiological pH. The anionic liposomes also can contain neutral co-lipids or cationic lipids, so long as the net charge of the liposome is negative and/or the surface of the liposome is negatively charged at physiological pH. In these embodiments, the ratio of anionic lipids to neutral co-lipids or cationic lipids is such that the overall charge of the resulting liposome is negative at physiological pH. For example, the anionic lipid is present in the anionic liposome at from about 10 mole % to about 100 mole % of total liposomal lipid, in some embodiments, from about 20 mole % to about 80 mole % and, in other embodiments, from about 20 mole % to about 60 mole %. The neutral lipid, when included in the anionic liposome, can be present at a concentration of from about 0 mole % to about 90 mole % of the total liposomal lipid, in some embodiments from about 20 mole % to about 80 mole %, and in other embodiments, from about 40 mole % to about 80 mole %. The positively charged lipid, when included in the anionic liposome, can be present at a concentration ranging from about 0 mole % to about 49 mole % of the total liposomal lipid, and in certain embodiments, from about 0 mole % to about 40 mole %.

In some embodiments in which the lipid vehicle is a cationic liposome or an anionic liposome, the delivery system complex as a whole has a net positive charge. By “net positive charge” is meant that the positive charges of the components of the delivery system complex exceed the negative charges of the components of the delivery system complex. It is to be understood, however, that the present invention also encompasses delivery system complexes having a positively charged surface irrespective of whether the net charge of the complex is positive, neutral or even negative. The charge of the surface of a delivery system complex can be measured by the migration of the complex in an electric field by methods known to those in the art, such as by measuring zeta potential (Martin, Swarick, and Cammarata (1983) Physical Pharmacy & Physical Chemical Principles in the Pharmaceutical Sciences, 3rd ed. Lea and Febiger) or by the binding affinity of the delivery system complex to cell surfaces. Complexes exhibiting a positively charged surface have a greater binding affinity to cell surfaces than complexes having a neutral or negatively charged surface. Further, it is to be understood that the positively charged surface can be sterically shielded by the addition of non-ionic polar compounds, for example, polyethylene glycol, as described elsewhere herein.

In particular non-limiting embodiments, the delivery system complex has a charge ratio of positive to negative charge (+: −) of between about 0.5:1 and about 100:1, including but not limited to about 0.5:1, about 1:1, about 2:1, about 3:1, about 4:1, about 5:1, about 6:1, about 7:1, about 8:1, about 9:1, about 10:1, about 15:1, about 20:1, about 40:1, or about 100:1. In a specific non-limiting embodiment, the +:− charge ratio is about 1:1.

The presently disclosed delivery system complexes can comprise liposomes that encapsulate a complex of dihydrate(1,2-diaminocyclohexane)platinum(II)-folinic acid precipitate that is in the core of the liposome. The release of the core contents can be sensitive to intracellular pH conditions within a cell or a cellular organelle. While not being bound by any particular theory or mechanism of action, it is believed the presently disclosed delivery system complexes enter cells through endocytosis and are found in endosomes, which exhibit a relatively low pH (e.g., pH 5.0). Thus, in some embodiments, the complex of dihydrate(1,2-diaminocyclohexane)platinum(II)-folinic acid precipitate readily dissolves at endosomal pH. In certain embodiments, the precipitate readily dissolves at a pH level of less than about 6.5, less than about 6.0, less than about 5.5, less than about 5.0, less than about 4.5, or less than about 4.0, including but not limited to, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, or less. In particular embodiments, the precipitate readily dissolves at a pH of 5.0 or less. In a preferred embodiment, a LCP-II nanoparticle comprises an acid-sensitive core. An acid-sensitive core dissolves more readily at pH levels below 7. In these embodiments, the LCP-II nanoparticle can unload more cargo at the target, e.g. the cytoplasm, than a nanoparticle formulated without an acid-sensitive core.

The delivery system complexes can be of any size, so long as the complex is capable of delivering the incorporated precipitate to a cell (e.g., in vitro, in vivo), physiological site, or tissue. In some embodiments, the delivery system complex is a nanoparticle, wherein the nanoparticle comprises a liposome encapsulating the precipitate, compound of Formula I. As used herein, the term “nanoparticle” refers to particles of any shape having at least one dimension that is less than about 1000 nm. In some embodiments, nanoparticles have at least one dimension in the range of about 1 nm to about 1000 nm, including any integer value between 1 nm and 1000 nm (including about 1, 2, 5, 10, 20, 30, 40, 50, 60, 70, 80, 90, 100, 200, 500, and 1000). In certain embodiments, the nanoparticles have at least one dimension that is about 120 nm. The polydispersity index can be from 0.2 to 0.4, such as 0.3. Particle size can be determined using any method known in the art, including, but not limited to, sedimentation field flow fractionation, photon correlation spectroscopy, disk centrifugation, and dynamic light scattering (using, for example, a submicron particle sizer such as the NICOMP particle sizing system from AutodilutePAT Model 370; Santa Barbara, Calif.).

As described elsewhere herein, the size of the delivery system complex can be regulated based on the ratio of non-ionic surfactant to organic solvent used during the generation of the water-in-oil microemulsion that comprises the precipitate. Further, the size of the delivery system complexes is dependent upon the ratio of the lipids in the liposome to the precipitate.

Methods for preparing liposomes are known in the art. For example, a review of methodologies of liposome preparation may be found in Liposome Technology (CFC Press NY 1984); Liposomes by Ostro (Marcel Dekker, 1987); Lichtenberg and Barenholz (1988) Methods Biochem Anal. 33:337-462 and U.S. Pat. No. 5,283,185, each of which are herein incorporated by reference in its entirety. For example, cationic lipids and optionally co-lipids can be emulsified by the use of a homogenizer, lyophilized, and melted to obtain multilamellar liposomes. Alternatively, unilamellar liposomes can be produced by the reverse phase evaporation method (Szoka and Papahadjopoulos (1978) Proc. Natl. Acad. Sci. USA 75:4194-4198, which is herein incorporated by reference in its entirety). In some embodiments, the liposomes are produced using thin film hydration (Bangham et al. (1965) J. Mol. Biol. 13:238-252, which is herein incorporated by reference in its entirety). In certain embodiments, the liposome formulation can be briefly sonicated and incubated at 50° C. for a short period of time (e.g., about 10 minutes) prior to sizing (see Templeton et al. (1997) Nature Biotechnology 15:647-652, which is herein incorporated by reference in its entirety).

An emulsion is a dispersion of one liquid in a second immiscible liquid. The term “immiscible” when referring to two liquids refers to the inability of these liquids to be mixed or blended into a homogeneous solution. Two immiscible liquids when added together will always form two separate phases. The organic solvent used in the presently disclosed methods is essentially immiscible with water. Emulsions are essentially swollen micelles, although not all micellar solutions can be swollen to form an emulsion. Micelles are colloidal aggregates of amphipathic molecules that are formed at a well-defined concentration known as the critical micelle concentration. Micelles are oriented with the hydrophobic portions of the lipid molecules at the interior of the micelle and the hydrophilic portions at the exterior surface, exposed to water. The typical number of aggregated molecules in a micelle (aggregation number) has a range from about 50 to about 100. The term “micelles” also refers to inverse or reverse micelles, which are formed in an organic solvent, wherein the hydrophobic portions are at the exterior surface, exposed to the organic solvent and the hydrophilic portion is oriented towards the interior of the micelle.

An oil-in-water (O/W) emulsion consists of droplets of an organic compound (e.g., oil) dispersed in water and a water-in-oil (W/O) emulsion is one in which the phases are reversed and is comprised of droplets of water dispersed in an organic compound (e.g., oil). A water-in-oil emulsion is also referred to herein as a reverse emulsion. Thermodynamically stable emulsions are those that comprise a surfactant (e.g, an amphipathic molecule) and are formed spontaneously. The term “emulsion” can refer to microemulsions or macroemulsions, depending on the size of the particles. Droplet diameters in microemulsions typically range from about 10 to about 100 nm. In contrast, the term macroemulsions refers to droplets having diameters greater than about 100 nm.

It will be evident to one of skill in the art that sufficient amounts of the aqueous solutions, organic solvent, and surfactants are added to the reaction solution to form the water-in-oil emulsion.

Surfactants are added to the reaction solution in order to facilitate the development of and stabilize the water-in-oil microemulsion. Surfactants are molecules that can reduce the surface tension of a liquid. Surfactants have both hydrophilic and hydrophobic properties, and thus, can be solubilized to some extent in either water or organic solvents. Surfactants are classified into four primary groups: cationic, anionic, non-ionic, and zwitterionic. The presently disclosed methods use non-ionic surfactants. Non-ionic surfactants are those surfactants that have no charge when dissolved or dispersed in aqueous solutions. Thus, the hydrophilic moieties of non-ionic surfactants are uncharged, polar groups. Representative non-limiting examples of non-ionic surfactants suitable for use for the presently disclosed methods and compositions include polyethylene glycol, polysorbates, including but not limited to, polyethoxylated sorbitan fatty acid esters (e.g., Tween® compounds) and sorbitan derivatives (e.g., Span® compounds); ethylene oxide/propylene oxide copolymers (e.g., Pluronic® compounds, which are also known as poloxamers); polyoxyethylene ether compounds, such as those of the Brij® family, including but not limited to polyoxyethylene stearyl ether (also known as polyoxyethylene (100) stearyl ether and by the trade name Brij® 700); ethers of fatty alcohols. In particular embodiments, the non-ionic surfactant comprises octyl phenol ethoxylate (i.e., Triton X-100), which is commercially available from multiple suppliers (e.g., Sigma-Aldrich, St. Louis, Mo.).

Polyethoxylated sorbitan fatty acid esters (polysorbates) are commercially available from multiple suppliers (e.g., Sigma-Aldrich, St Louis, Mo.) under the trade name Tween®, and include, but are not limited to, polyoxyethylene (POE) sorbitan monooleate (Tween® 80), POE sorbitan monostearate (Tween® 60), POE sorbitan monolaurate (Tween® 20), and POE sorbitan monopalmitate (Tween® 40).

Ethylene oxide/propylene oxide copolymers include the block copolymers known as poloxamers, which are also known by the trade name Pluronic® and can be purchased from BASF Corporation (Florham Park, N.J.). Poloxamers are composed of a central hydrophobic chain of polyoxypropylene (poly(propylene oxide)) flanked by two hydrophilic chains of polyoxyethylene (poly(ethylene oxide)) and are represented by the following chemical structure: HO(C₂H₄O)ₐ(C₃H₆O)_{b}(C₂H₄O)ₐH; wherein the C₂H₄O subunits are ethylene oxide monomers and the C₃H₆O subunits are propylene oxide monomers, and wherein a and b can be any integer ranging from 20 to 150.

Organic solvents that can be used in the presently disclosed methods include those that are immiscible or essentially immiscible with water. Non-limiting examples of organic solvents that can be used in the presently disclosed methods include chloroform, methanol, ether, ethyl acetate, hexanol, cyclohexane, and dichloromethane. In particular embodiments, the organic solvent is nonpolar or essentially nonpolar.

In some embodiments, mixtures of more than one organic solvent can be used in the presently disclosed methods. In some of these embodiments, the organic solvent comprises a mixture of cyclohexane and hexanol. In particular embodiments, the organic solvent comprises cyclohexane and hexanol at a volume/volume ratio of about 7.5:1.7. As noted elsewhere herein, the non-ionic surfactant can be added to the reaction solution (comprising aqueous solutions of cation, anion, bioactive compound of Formula I, and organic solvent) separately, or it can first be mixed with the organic solvent and the organic solvent/surfactant mixture can be added to the aqueous solutions of the anion, cation, and bioactive compound of Formula I. In some of these embodiments, a mixture of cyclohexane, hexanol, and Triton X-100 is added to the reaction solution. In particular embodiments, the volume/volume/volume ratio of the cyclohexane:hexanol:Triton X-100 of the mixture that is added to the reaction solution is about 7.5:1.7:1.8.

It should be noted that the volume/volume ratio of the nonionic surfactant to the organic solvent regulates the size of the water-in-oil microemulsion and therefore, the precipitate contained therein and the resultant delivery system complex, with a greater surfactant:organic solvent ratio resulting in delivery system complexes with larger diameters and smaller surfactant:organic solvent ratios resulting in delivery system complexes with smaller diameters.

The reaction solution may be mixed to form the water-in-oil microemulsion and the solution may also be incubated for a period of time. This incubation step can be performed at room temperature. In some embodiments, the reaction solution is mixed at room temperature for a period of time of between about 5 minutes and about 60 minutes, including but not limited to about 5 minutes, about 10 minutes, about 15 minutes, about 20 minutes, about 25 minutes, about 30 minutes, about 35 minutes, about 40 minutes, about 45 minutes, about 50 minutes, about 55 minutes, and about 60 minutes. In particular embodiments, the reaction solution is mixed at room temperature for about 15 minutes.

In order to complex the precipitate with a liposome, the surface of the precipitate can be modified. In some embodiments, the precipitate is neutral following its formation. In some embodiments, the precipitate will have a charged surface following its formation. Those precipitates with positively charged surfaces can be mixed with anionic liposomes, whereas those precipitates with negatively charged surfaces can be mixed with cationic liposomes. In particular, the complex of OxP-FnA, is neutral and can be stabilized by an amphiphilic lipid, such as DOPA. In certain embodiments, the stabilized complex of OxP-FnA is coated with a cationic lipid, such as DOTAP, to prepare a nano-Folox particle. The term “stabilized” refers to a precipitate that is capable of being coated by a second lipid coating.

In some embodiments, the nano-Folox particle has or is modified to have a zeta potential of less than −10 mV and in certain embodiments, the zeta potential is between about −1 mV and about −10 mV, including but not limited to about −4 mV, about −5 mV, and about −6 mV. In particular embodiments, the zeta potential of the precipitate is about −16 mV.

In certain embodiments, the outer leaflet is comprised of different lipids rather than a single, relatively pure lipid. This also referred to herein as an asymmetric lipid membrane. The asymmetric lipid membrane can shield the charges that would be present on a pure liposome. Preferably, a positive zeta potential is of a lower value than the pure liposome.

Following the production of the water-in-oil emulsion, the precipitate can be purified from the non-ionic surfactant and organic solvent. The precipitate can be purified using any method known in the art, including but not limited to gel filtration chromatography. A precipitate that has been purified from the non-ionic surfactants and organic solvent is a precipitate that is essentially free of non-ionic surfactants or organic solvents (e.g, the precipitate comprises less than 10%, less than 1%, less than 0.1% by weight of the non-ionic surfactant or organic solvent). In some of those embodiments wherein gel filtration is used to purify the precipitate, the precipitate is adsorbed to a silica gel or to a similar type of a stationary phase, the silica gel or similar stationary phase is washed with a polar organic solvent (e.g., ethanol, methanol, acetone, DMSO, DMF) to remove the non-ionic surfactant and organic solvent, and precipitate is eluted from the silica gel or other solid surface with an aqueous solution comprising a polar organic solvent.

In some of these embodiments, the silica gel is washed with ethanol and the precipitate is eluted with a mixture of water and ethanol. In particular embodiments, the precipitate is eluted with a mixture of water and ethanol, wherein the mixture comprises a volume/volume ratio of between about 1:9 and about 1:1, including but not limited to, about 1:9, about 1:8, about 1:7, about 1:6, about 1:5, about 1:4, about 1:3, about 1:2, and about 1:1. In particular embodiments, the volume/volume ratio of water to ethanol is about 1:3. In some of these embodiments, a mixture comprising 25 ml water and 75 ml ethanol is used for the elution step. Following removal of the ethanol using, for example, rotary evaporation, the precipitate can be dispersed in an aqueous solution (e.g., water) prior to mixing with the prepared liposomes.

In certain embodiments, the methods of making the delivery system complexes can further comprise an additional purification step following the production of the delivery system complexes, wherein the delivery system complexes are purified from excess free liposomes and unencapsulated precipitates. Purification can be accomplished through any method known in the art, including, but not limited to, centrifugation through a sucrose density gradient or other media which is suitable to form a density gradient. It is understood, however, that other methods of purification such as chromatography, filtration, phase partition, precipitation or absorption can also be utilized. In one method, purification via centrifugation through a sucrose density gradient is utilized. The sucrose gradient can range from about 0% sucrose to about 60% sucrose or from about 5% sucrose to about 30% sucrose. The buffer in which the sucrose gradient is made can be any aqueous buffer suitable for storage of the fraction containing the complexes and in some embodiments, a buffer suitable for administration of the complex to cells and tissues.

In some embodiments, a targeted delivery system or a PEGylated delivery system is made as described elsewhere herein, wherein the methods further comprise a post-insertion step following the preparation of the liposome or following the production of the delivery system complex, wherein a lipid-targeting ligand conjugate or a PEGylated lipid is post-inserted into the liposome. Liposomes or delivery system complexes comprising a lipid-targeting ligand conjugate or a lipid-PEG conjugate can be prepared following techniques known in the art, including but not limited to those presented herein (see Experimental section; Ishida et al. (1999) FEBS Lett. 460:129-133; Perouzel et al. (2003) Bioconjug. Chem. 14:884-898, which is herein incorporated by reference in its entirety). The post-insertion step can comprise mixing the liposomes or the delivery system complexes with the lipid-targeting ligand conjugate or a lipid-PEG conjugate and incubating the particles at about 50° C. to about 60° C. for a brief period of time (e.g., about 5 minutes, about 10 minutes). In some embodiments, the delivery system complexes or liposomes are incubated with a lipid-PEG conjugate or a lipid-PEG-targeting ligand conjugate at a concentration of about 5 to about 20 mol %, including but not limited to about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, and about 20 mol %, to form a stealth delivery system. In some of these embodiments, the concentration of the lipid-PEG conjugate is about 10 mol %. The polyethylene glycol moiety of the lipid-PEG conjugate can have a molecular weight ranging from about 100 to about 20,000 g/mol, including but not limited to about 100 g/mol, about 200 g/mol, about 300 g/mol, about 400 g/mol, about 500 g/mol, about 600 g/mol, about 700 g/mol, about 800 g/mol, about 900 g/mol, about 1000 g/mol, about 5000 g/mol, about 10,000 g/mol, about 15,000 g/mol, and about 20,000 g/mol. In certain embodiments, the lipid-PEG conjugate comprises a PEG molecule having a molecular weight of about 2000 g/mol. In some embodiments, the lipid-PEG conjugate comprises DSPE-PEG₂₀₀₀. Lipid-PEG-targeting ligand conjugates can also be post-inserted into liposomes or delivery system complexes using the above described post-insertion methods.

As described elsewhere herein, the delivery system complexes can have a surface charge (e.g., positive charge). In some embodiments, the surface charge of the liposome of the delivery system can be minimized by incorporating lipids comprising polyethylene glycol (PEG) moieties into the liposome. Reducing the surface charge of the liposome of the delivery system can reduce the amount of aggregation between the delivery system complexes and serum proteins and enhance the circulatory half-life of the complex (Yan, Scherphof, and Kamps (2005) J Liposome Res 15:109-139). Thus, in some embodiments, the exterior surface of the liposome or the outer leaflet of the lipid bilayer of the delivery system comprises a PEG molecule. Such a complex is referred to herein as a PEGylated delivery system complex. In these embodiments, the outer leaflet of the lipid bilayer of the liposome of the delivery system complex comprises a lipid-PEG conjugate.

A PEGylated delivery system complex can be generated through the post-insertion of a lipid-PEG conjugate into the lipid bilayer through the incubation of the delivery system complex with micelles comprising lipid-PEG conjugates, as known in the art and described elsewhere herein (Ishida et al. (1999) FEBS Lett. 460:129-133; Perouzel et al. (2003) Bioconjug. Chem. 14:884-898; see Experimental section). By “lipid-polyethylene glycol conjugate” or “lipid-PEG conjugate” is intended a lipid molecule that is covalently bound to at least one polyethylene glycol molecule. In some embodiments, the lipid-PEG conjugate comprises 1,2-distearoyl-sn-glycero-3-phosphoethanolamine-N-carboxy-polyethylene glycol (DSPE-PEG). As described immediately below, these lipid-PEG conjugates can be further modified to include a targeting ligand, forming a lipid-PEG-targeting ligand conjugate (e.g., DSPE-PEG-AA). The term “lipid-PEG conjugate” also refers to these lipid-PEG-targeting ligand conjugates and a delivery system complex comprising a liposome comprising a lipid-PEG targeting ligand conjugate are considered to be both a PEGylated delivery system complex and a targeted delivery system complex, as described immediately below.

Alternatively, the delivery system complex can be PEGylated through the addition of a lipid-PEG conjugate during the formation of the outer leaflet of the lipid bilayer.

PEGylation of liposomes enhances the circulatory half-life of the liposome by reducing clearance of the complex by the reticuloendothelial (RES) system. While not being bound by any particular theory or mechanism of action, it is believed that a PEGylated delivery system complex can evade the RES system by sterically blocking the opsonization of the complexes (Owens and Peppas (2006) Int J Pharm 307:93-102). In order to provide enough steric hindrance to avoid opsonization, the exterior surface of the liposome must be completely covered by PEG molecules in the “brush” configuration. At low surface coverage, the PEG chains will typically have a “mushroom” configuration, wherein the PEG molecules will be located closer to the surface of the liposome. In the “brush” configuration, the PEG molecules are extended further away from the liposome surface, enhancing the steric hindrance effect. However, over-crowdedness of PEG on the surface may decrease the mobility of the polymer chains and thus decrease the steric hindrance effect (Owens and Peppas (2006) Int J Pharm 307:93-102).

The conformation of PEG depends upon the surface density and the molecular mass of the PEG on the surface of the liposome. The controlling factor is the distance between the PEG chains in the lipid bilayer (D) relative to their Flory dimension, R_{F}, which is defined as aN³, wherein a is the persistence length of the monomer, and N is the number of monomer units in the PEG (see Nicholas et al. (2000) Biochim Biophys Acta 1463:167-178, which is herein incorporated by reference). Three regimes can be defined: (1) when D>2 R_{F} (interdigitated mushrooms); (2) when D<2 R_{F} (mushrooms); and (3) when D<R_{F} (brushes) (Nicholas et al.).

In certain embodiments, the PEGylated delivery system complex comprises a stealth delivery system complex. By “stealth delivery system complex” is intended a delivery system complex comprising a liposome wherein the outer leaflet of the lipid bilayer of the liposome comprises a sufficient number of lipid-PEG conjugates in a configuration that allows the delivery system complex to exhibit a reduced uptake by the RES system in the liver when administered to a subject as compared to non PEGylated delivery system complexes. RES uptake can be measured using assays known in the art, including, but not limited to the liver perfusion assay described in International Application No. PCT/US2009/042485, filed on May 1, 2009. In some of these embodiments, the stealth delivery system complex comprises a liposome, wherein the outer leaflet of the lipid bilayer of the liposome comprises PEG molecules, wherein said D<R_{F}.

In some of those embodiments in which the PEGylated delivery system is a stealth polynucleotide system, the outer leaflet of the lipid bilayer of the cationic liposome comprises a lipid-PEG conjugate at a concentration of about 4 mol % to about 15 mol % of the outer leaflet lipids, including, but not limited to, about 4 mol %, about 5 mol %, about 6 mol %, about 7 mol %, 8 mol %, about 9 mol %, about 10 mol %, about 11 mol %, about 12 mol %, about 13 mol %, about 14 mol %, and about 15 mol % PEG. In certain embodiments, the outer leaflet of the lipid bilayer of the cationic liposome of the stealth delivery system complex comprises about 10.6 mol % PEG. Higher percentage values (expressed in mol %) of PEG have also surprisingly been found to be useful. Useful mol % values include those from about 12 mol % to about 50 mol %. Preferably, the values are from about 15 mol % to about 40 mol %. Also preferred are values from about 15 mol % to about 35 mol %. Most preferred values are from about 20 mol % to about 25 mol %, for example 23 mol %.

The polyethylene glycol moiety of the lipid-PEG conjugate can have a molecular weight ranging from about 100 to about 20,000 g/mol, including but not limited to about 100 g/mol, about 200 g/mol, about 300 g/mol, about 400 g/mol, about 500 g/mol, about 600 g/mol, about 700 g/mol, about 800 g/mol, about 900 g/mol, about 1000 g/mol, about 5000 g/mol, about 10,000 g/mol, about 15,000 g/mol, and about 20,000 g/mol. In some embodiments, the lipid-PEG conjugate comprises a PEG molecule having a molecular weight of about 2000 g/mol. In certain embodiments, the lipid-PEG conjugate comprises DSPE-PEG_{2000.}

In some embodiments, the delivery system complex comprises a liposome, wherein the exterior surface of the liposome, or the delivery system complex comprises a lipid bilayer wherein the outer leaflet of the lipid bilayer, comprises a targeting ligand, thereby forming a targeted delivery system. In these embodiments, the outer leaflet of the liposome comprises a targeting ligand. By “targeting ligand” is intended a molecule that targets a physically associated molecule or complex to a targeted cell or tissue. As used herein, the term “physically associated” refers to either a covalent or non-covalent interaction between two molecules. A “conjugate” refers to the complex of molecules that are covalently bound to one another. For example, the complex of a lipid covalently bound to a targeting ligand can be referred to as a lipid-targeting ligand conjugate.

Alternatively, the targeting ligand can be non-covalently bound to a lipid. “Non-covalent bonds” or “non-covalent interactions” do not involve the sharing of pairs of electrons, but rather involve more dispersed variations of electromagnetic interactions, and can include hydrogen bonding, ionic interactions, Van der Waals interactions, and hydrophobic bonds.

Targeting ligands can include, but are not limited to, small molecules, peptides, lipids, sugars, oligonucleotides, hormones, vitamins, antigens, antibodies or fragments thereof, specific membrane-receptor ligands, ligands capable of reacting with an anti-ligand, fusogenic peptides, nuclear localization peptides, or a combination of such compounds. Non-limiting examples of targeting ligands include asialoglycoprotein, insulin, low density lipoprotein (LDL), folate, benzamide derivatives, peptides comprising the arginine-glycine-aspartate (RGD) sequence, and monoclonal and polyclonal antibodies directed against cell surface molecules. In some embodiments, the small molecule comprises a benzamide derivative. In some of these embodiments, the benzamide derivative comprises anisamide.

The targeting ligand can be covalently bound to the lipids comprising the liposome or lipid bilayer of the delivery system, including a cationic lipid, or a co-lipid, forming a lipid-targeting ligand conjugate. As described above, a lipid-targeting ligand conjugate can be post-inserted into the lipid bilayer of a liposome using techniques known in the art and described elsewhere herein (Ishida et al. (1999) FEBS Lett. 460:129-133; Perouzel et al. (2003) Bioconjug. Chem. 14:884-898; see Experimental section). Alternatively, the lipid-targeting ligand conjugate can be added during the formation of the outer leaflet of the lipid bilayer.

Some lipid-targeting ligand conjugates comprise an intervening molecule in between the lipid and the targeting ligand, which is covalently bound to both the lipid and the targeting ligand. In some of these embodiments, the intervening molecule is polyethylene glycol (PEG), thus forming a lipid-PEG-targeting ligand conjugate. An example of such a lipid-targeting conjugate is DSPE-PEG-AA, in which the lipid 1,2-distearoyl-sn-glycero-3-phosphoethanolamine-N-carboxyl (DSPE) is bound to polyethylene glycol (PEG), which is bound to the targeting ligand anisamide (AA). Thus, in some embodiments, the cationic lipid vehicle of the delivery system comprises the lipid-targeting ligand conjugate DSPE-PEG-AA.

By “targeted cell” is intended the cell to which a targeting ligand recruits a physically associated molecule or complex. The targeting ligand can interact with one or more constituents of a target cell. The targeted cell can be any cell type or at any developmental stage, exhibiting various phenotypes, and can be in various pathological states (i.e., abnormal and normal states). For example, the targeting ligand can associate with normal, abnormal, and/or unique constituents on a microbe (i.e., a prokaryotic cell (bacteria), viruses, fungi, protozoa or parasites) or on a eukaryotic cell (e.g., epithelial cells, muscle cells, nerve cells, sensory cells, cancerous cells, secretory cells, malignant cells, erythroid and lymphoid cells, stem cells). Thus, the targeting ligand can associate with a constitutient on a target cell which is a disease-associated antigen including, for example, tumor-associated antigens and autoimmune disease-associated antigens. Such disease-associated antigens include, for example, growth factor receptors, cell cycle regulators, angiogenic factors, and signaling factors.

In some embodiments, the targeting ligand interacts with a cell surface protein on the targeted cell. In some of these embodiments, the expression level of the cell surface protein that is capable of binding to the targeting ligand is higher in the targeted cell relative to other cells. For example, cancer cells overexpress certain cell surface molecules, such as the HER2 receptor (breast cancer) or the sigma receptor. In certain embodiments wherein the targeting ligand comprises a benzamide derivative, such as anisamide, the targeting ligand targets the associated delivery system complex to sigma-receptor overexpressing cells, which can include, but are not limited to, cancer cells such as small- and non-small-cell lung carcinoma, renal carcinoma, colon carcinoma, sarcoma, breast cancer, melanoma, glioblastoma, neuroblastoma, and prostate cancer (Aydar, Palmer, and Djamgoz (2004) Cancer Res. 64:5029-5035).

Thus, in some embodiments, the targeted cell comprises a cancer cell. The terms “cancer” or “cancerous” refer to or describe the physiological condition in mammals that is typically characterized by unregulated cell growth. As used herein, “cancer cells” or “tumor cells” refer to the cells that are characterized by this unregulated cell growth. The term “cancer” encompasses all types of cancers, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer. In some embodiments, the targeted cancer cell comprises a colorectal cancer (CRC) cell.

In certain embodiments, the subject matter described herein is directed to methods of making the delivery system complex, said method comprising:
a) contacting one or more single-lipid layer cores comprising nano-Folox, one or more single-lipid layer cores comprising FdUMP, and a polymer, such as PLGA, PLGA-PEG, and/or PLGA-PEG-AEAA (for example all three are present at a ratio of about 4:4:2) in a solvent, such as THF to form a solution;
b) contacting the solution with water to form a suspension; and
c) stirring the suspension; wherein a delivery system complex is prepared.

In certain embodiments, the subject matter described herein is directed to methods of making the delivery system complex, said method comprising:
a) contacting one or more single-lipid layer cores comprising nano-Folox; one or more single-lipid layer cores comprising FdUMP; a polymer, such as PLGA, PLGA-PEG, and/or PLGA-PEG-AEAA (for example all three are present at a ratio of about 4:4:2); and SN-38, in a solvent, such as THF to form a solution;
b) contacting the solution with water to form a suspension; and
c) stirring the suspension; wherein a delivery system complex is prepared.

In certain embodiments, the subject matter described herein is directed to methods of making the delivery system complex, said method comprising:
a) preparing a precipitate of dihydrate(1,2-diaminocyclohexane)platinum(II) ([Pt(DACH)(H₂O)₂]²⁺, and folinic acid;
b) contacting said precipitate with an amphiphilic lipid to stabilize;
c) contacting the stabilized precipitate with a cationic lipid to prepare said delivery system complex.

### II. Pharmaceutical Compositions and Methods of Delivery and Treatment

In certain embodiments, the subject matter described herein is directed to a method treating cancer comprising, administering to a subject an effective amount of the compound of Formula I or the delivery system complex comprising Formula I as described herein. The compound or the delivery system complex can be formulated with excipients for administration.

In certain embodiments, the method of treatment further comprises administering a second active agent before, after or concurrently with said delivery system complex. In certain embodiments, the second active agent is an antimetabolite chemotherapeutic drug or a monoclonal antibody. In certain embodiments, the antimetabolite chemotherapeutic drug is 5-fluorouracil or Nano-FdUMP. In certain embodiments, the monoclonal antibody is anti-PD-L1 antibody. The method of administering and dosages for each are within the purview of those of skill in the art, or are known in the art.

In certain embodiments, the cancer is colorectal cancer.

The delivery system complexes described herein are useful in mammalian tissue culture systems, in animal studies, and for therapeutic purposes. The delivery system complexes have been shown to have therapeutic activity when introduced into a cell or tissue. The delivery system complexes can be administered for therapeutic purposes or pharmaceutical compositions comprising the delivery system complexes along with additional pharmaceutical carriers can be formulated for delivery, i.e., administering to the subject, by any available route including, but not limited, to parenteral (e.g., intravenous), intradermal, subcutaneous, oral, nasal, bronchial, opthalmic, transdermal (topical), transmucosal, rectal, and vaginal routes. In some embodiments, the route of delivery is intravenous, parenteral, transmucosal, nasal, bronchial, vaginal, and oral.

As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds also can be incorporated into the compositions.

As one of ordinary skill in the art would appreciate, a presently disclosed pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions or dispersions such as those described elsewhere herein and sterile powders for the extemporaneous preparation of sterile injectable solutions or dispersions. For intravenous administration, suitable carriers include physiological saline, bacteriostatic water, or phosphate buffered saline (PBS). The composition should be sterile and should be fluid to the extent that easy syringability exists. In some embodiments, the pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols, such as manitol or sorbitol, or sodium chloride are included in the formulation. Prolonged absorption of the injectable formulation can be brought about by including in the formulation an agent that delays absorption, for example, aluminum monostearate and gelatin.

Sterile injectable solutions can be prepared by filter sterilization as described elsewhere herein. In certain embodiments, solutions for injection are free of endotoxin. Generally, dispersions are prepared by incorporating the delivery system complexes into a sterile vehicle which contains a basic dispersion medium and the required other ingredients from those enumerated above. In those embodiments in which sterile powders are used for the preparation of sterile injectable solutions, the solutions can be prepared by vacuum drying and freeze-drying which yields a powder of the active ingredient plus any additional desired ingredient from a previously sterile-filtered solution thereof.

Oral compositions generally include an inert diluent or an edible carrier. Oral compositions can be prepared using a fluid carrier for use as a mouthwash. Pharmaceutically compatible binding agents, and/or adjuvant materials can be included as part of the composition. The oral compositions can include a sweetening agent, such as sucrose or saccharin; or a flavoring agent, such as peppermint, methyl salicylate, or orange flavoring.

For administration by inhalation, the presently disclosed compositions can be delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical or cosmetic carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals. Guidance regarding dosing is provided elsewhere herein.

The present subject matter also includes an article of manufacture providing a delivery system complex described herein. The article of manufacture can include a vial or other container that contains a composition suitable for the present method together with any carrier, either dried or in liquid form. The article of manufacture further includes instructions in the form of a label on the container and/or in the form of an insert included in a box in which the container is packaged, for carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

The present subject matter provides methods for delivering a bioactive compound of Formula I to a cell and for treating a disease or unwanted condition in a subject with a delivery system complex comprising a bioactive compound of Formula I that has therapeutic activity against the disease or unwanted condition. Further provided herein are methods for making the presently disclosed delivery system complexes.

The presently disclosed delivery system complexes can be used to deliver the bioactive compound of Formula I to cells by contacting a cell with the delivery system complexes. As described elsewhere herein, the term “deliver” when referring to a bioactive compound of Formula I refers to the process resulting in the placement of the composition within the intracellular space of the cell or the extracellular space surrounding the cell. The term “cell” encompasses cells that are in culture and cells within a subject. In these embodiments, the cells are contacted with the delivery system complex in such a manner as to allow the precipitate comprised within the delivery system complexes to gain access to the interior of the cell.

The delivery of a bioactive compound of Formula I to a cell can comprise an in vitro approach, an ex vivo approach, in which the delivery of the bioactive compound of Formula I into a cell occurs outside of a subject (the transfected cells can then be transplanted into the subject), and an in vivo approach, wherein the delivery occurs within the subject itself.

The compound of Formula I or nano-Folox is administered to the subject in a therapeutically effective amount. By “therapeutic activity” when referring to a compound or nano-Folox is intended that the compound or nano-Folox is able to elicit a desired pharmacological or physiological effect when administered to a subject in need thereof.

As used herein, the terms “treatment” or “prevention” refer to obtaining a desired pharmacologic and/or physiologic effect. The effect may be prophylactic in terms of completely or partially preventing a particular infection or disease or sign or symptom thereof and/or may be therapeutic in terms of a partial or complete cure of an infection or disease and/or adverse effect attributable to the infection or the disease. Accordingly, the method “prevents” (i.e., delays or inhibits) and/or “reduces” (i.e., decreases, slows, or ameliorates) the detrimental effects of a disease or disorder in the subject receiving the compositions of the invention. The subject may be any animal, including a mammal, such as a human, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The disease or unwanted condition to be treated can encompass any type of condition or disease that can be treated therapeutically. In some embodiments, the disease or unwanted condition that is to be treated is a cancer. As described elsewhere herein, the term “cancer” encompasses any type of unregulated cellular growth and includes all forms of cancer. In some embodiments, the cancer to be treated is a lung cancer. Methods to detect the inhibition of cancer growth or progression are known in the art and include, but are not limited to, measuring the size of the primary tumor to detect a reduction in its size, delayed appearance of secondary tumors, slowed development of secondary tumors, decreased occurrence of secondary tumors, and slowed or decreased severity of secondary effects of disease.

It will be understood by one of skill in the art that the delivery system complexes can be used alone or in conjunction with other therapeutic modalities, including, but not limited to, surgical therapy, radiotherapy, or treatment with any type of therapeutic agent, such as a drug. In those embodiments in which the subject is afflicted with cancer, the delivery system complexes can be delivered in combination with any chemotherapeutic agent well known in the art.

When administered to a subject in need thereof, the delivery system complexes can further comprise a targeting ligand, as discussed elsewhere herein. In these embodiments, the targeting ligand will target the physically associated complex to a targeted cell or tissue within the subject. In certain embodiments, the targeted cell or tissue comprises a diseased cell or tissue or a cell or tissue characterized by the unwanted condition. In some of these embodiments, the delivery system complex is a stealth delivery system complex wherein the surface charge is shielded through the association of PEG molecules and the liposome further comprises a targeting ligand to direct the delivery system complex to targeted cells.

In some embodiments, particularly those in which the diameter of the delivery system complex is less than 100 nm, the delivery system complexes can be used to deliver a compound of Formula I across the blood-brain barrier (BBB) into the central nervous system or across the placental barrier. Non-limiting examples of targeting ligands that can be used to target the BBB include transferring and lactoferrin (Huang et al. (2008) Biomaterials 29(2):238-246, which is herein incorporated by reference in its entirety). Further, the delivery system complexes can be transcytosed across the endothelium into both skeletal and cardiac muscle cells. For example, exon-skipping oligonucleotides can be delivered to treat Duchene muscular dystrophy (Moulton et al. (2009) Ann N Y Acad Sci 1175:55-60, which is herein incorporated by reference in its entirety).

Delivery of a therapeutically effective amount of a delivery system complex comprising a compound of Formula I can be obtained via administration of a pharmaceutical composition comprising a therapeutically effective dose of the compound of Formula I or the delivery system complex. By “therapeutically effective amount” or “dose” is meant the concentration of a delivery system or a compound of Formula I comprised therein that is sufficient to elicit the desired therapeutic effect.

As used herein, “effective amount” is an amount sufficient to effect beneficial or desired clinical or biochemical results. An effective amount can be administered one or more times.

The effective amount of the delivery system complex or compound of Formula I will vary according to the weight, sex, age, and medical history of the subject. Other factors which influence the effective amount can include, but are not limited to, the severity of the subject's condition, the disorder being treated, the stability of the compound or complex, and, if desired, the adjuvant therapeutic agent being administered along with the polynucleotide delivery system. Methods to determine efficacy and dosage are known to those skilled in the art. See, for example, Isselbacher et al. (1996) Harrison's Principles of Internal Medicine 13 ed., 1814-1882, herein incorporated by reference.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic (e.g., immunotoxic) and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the presently disclosed methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical formulation can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, and the like. The skilled artisan will appreciate that certain factors can influence the dosage and timing required to effectively treat a subject, including but not limited to the severity of the disease, disorder, or unwanted condition, previous treatments, the general health and/or age of the subject, and other diseases or unwanted conditions present. Generally, treatment of a subject can include a single treatment or, in many cases, can include a series of treatments. Further, treatment of a subject can include a single cosmetic application or, in some embodiments, can include a series of cosmetic applications.

It is understood that appropriate doses of a compound depend upon its potency and can optionally be tailored to the particular recipient, for example, through administration of increasing doses until a preselected desired response is achieved. It is understood that the specific dose level for any particular animal subject can depend on a variety of factors including the activity of the specific compound employed, the age, body weight, general health, gender, and diet of the subject, the time of administration, the route of administration, the rate of excretion, any drug combination, and the degree of expression or activity to be modulated.

One of ordinary skill in the art upon review of the presently disclosed subject matter would appreciate that the presently disclosed compound of Formula I and nano-Folox and pharmaceutical compositions thereof, can be administered directly to a cell, a cell culture, a cell culture medium, a tissue, a tissue culture, a tissue culture medium, and the like. When referring to the delivery systems of the invention, the term “administering,” and derivations thereof, comprises any method that allows for the compound to contact a cell. The presently disclosed compounds or pharmaceutical compositions thereof, can be administered to (or contacted with) a cell or a tissue in vitro or ex vivo. The presently disclosed compounds or pharmaceutical compositions thereof, also can be administered to (or contacted with) a cell or a tissue in vivo by administration to an individual subject, e.g., a patient, for example, by systemic administration (e.g., intravenous, intraperitoneal, intramuscular, subdermal, or intracranial administration) or topical application, as described elsewhere herein.

It is to be noted that the term “a” or “an” entity refers to one or more of that entity; for example, “a nanoparticle” is understood to represent one or more nanoparticles. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

The following examples are offered by way of illustration and not by way of limitation.

### EXAMPLES

### Materials and Methods for Examples 1-9

Materials. N-(Methoxypolyethylene oxycarbonyl)-1,2-distearoryl-sn-glycero-3-phosphoethanolamine (DSPE-PEG; SUNBRIGHT® DSPE-020CN) was obtained from NOF CORPORATION. N-(2-aminoethyl)-4-methoxybenzamide conjugated DSPE-PEG (DSPE-PEG-AEAA) was synthesized as previously described in our laboratory⁵⁸. 1,2-dioleoyl-sn-glycero-3-phosphate (DOPA) and 1,2-dioleoyl-3-trimethylammonium-propane (DOTAP) were obtained from Avanti Polar Lipids, Inc. Oxaliplatin (OxP) was obtained from Selleckchem. Dichloro(1,2-diaminocyclohexane)platinum(II), folinic acid (FnA), cyclohexane, Triton X-100 and hexanol, silver nitrate (AgNO₃), cholesterol and bovine serum albumin (BSA) were purchased from Sigma-Aldrich. All chemicals were used as received without any further purification.

Cell culture. CT26-FL3 cells stably expressing red fluorescent protein/Luc³⁰, a mouse CRC cell line kindly provided by Dr. Maria Pena at the University of South Carolina, were maintained in Dulbecco's Modified Eagle's Medium (DMEM, high glucose, Gibco) supplemented with 10% fetal bovine serum (Gibco), 1% antibiotic-antimycotic (Gibco) and 1 μg/mL puromycin (ThermoFisher). Cells were grown at 37° C. with 5% CO₂ and 95% relative humidity.

In vitro characterization of Nano-Folox. Cytotoxicity of Nano-Folox was estimated using the MTT assay with 3-(4,5-Dimethylthiazol-2-yl)-2,5-diphenyltetrazolium bromide⁶⁰. 10,000 CT26-FL3 cells per well were seeded in 96-well plates and incubated for one day. After incubation, cells were added with OxP and Nano-Folox for 24 h under normal growth conditions. Subsequently, cells were added with 20 μL MTT reagent (5 mg/mL in PBS) and incubated for 4 h at 37° C. 150 μL DMSO were used to dissolve the purple formazan products. The results were measured at 570 nm using a microplate reader. The 50% cell growth inhibition (IC50) was estimated using the GraphPad Prism software.

CT26-FL3 cells were also seeded in 24-well plates at a density of 5×10⁴ cells per well for 24 h. Subsequently, cells were incubated with OxP ([c] of platinum=10 μM) and Nano-Folox for 4 h under normal growth conditions. After incubation, cells were washed twice with PBS and lysed for ICP-MS analysis in order to determine the uptake of platinum.

In addition, 5×10⁴ CT26-FL3 cells per well were seeded in 24-well plates for one day. After this, cells were incubated with OxP ([c] of platinum=10 μM) and Nano-Folox for 24 h under normal growth conditions. Following incubation, cells were treated with Annexin V-FTIC and propidium iodide (PI) according to the manufacturer's instructions (ThermoFisher). The apoptotic cells were analyzed using the Becton Dickinson LSR II.

Immunogenic cell death (ICD) in terms of CRT exposure and HMGB1 release was determined as previously described⁶¹. Briefly, 5×10⁴ CT26-FL3 cells per well were seeded in 8-well chamber slides (Nunc™ Lab-Tek™ II CC2™ Chamber Slide System, ThermoFisher) for one day. Subsequently, cells were treated with OxP ([c] of platinum=10 μM) and Nano-Folox under normal growth conditions. Following 2 h incubation, cells were washed with PBS and fixed with 0.25% paraformaldehyde (PFA) in PBS for 5 min. Cells were then washed with PBS, and the anti-Calreticulin (CRT) primary antibody (ab2907, Abcam) was applied for 1 h. Following two PBS washes, cells were incubated with the FITC-conjugated secondary antibody (ab150077, Abcam) for 30 min. Cells were then fixed with 4% PFA for 20 min, and were stained with ProLong™ Gold Antifade Mountant with DAPI (ThermoFisher) before the confocal imaging (Zeiss LSM 710). In addition, following 8 h incubation, cells were washed with PBS, fixed with 4% PFA for 20 min, and permeabilized with 0.1% Triton X-100 for 10 min. Following two PBS washes, cells were incubated with 1% BSA for 30 min. Cells were then washed with PBS and incubated with the primary antibody against the high-mobility group box 1 protein (HMGB1) (ab18256, Abcam) for 1 h. After this, cells were washed with PBS and incubated with the FITC-conjugated secondary antibody (ab150077, Abcam) for 30 min before the confocal analysis.

Pharmacokinetics and biodistribution. Female BALB/C mice (˜6 weeks) were purchased from Charles River Laboratories. All animal regulations and procedures were accepted by Institutional Animal Care and Use Committee of University of North Carolina at Chapel Hill.

The orthotopic CT26-FL3 colorectal tumor model was established as previously described³¹. After tumor inoculation (Day 0), mice were intraperitoneally (i.p.) injected with 100 μL of 10 mg/mL D-luciferin (Pierce™), and the tumor development was regularly monitored by the bioluminescent analysis using an IVIS® Kinetics Optical System (Perkin Elmer, CA). When the luminescence intensities reached ˜1×10⁹ p/sec/cm²/sr (Day 14), pharmacokinetics and tissue distribution studies were performed as follows.

Tumor-bearing mice (n=4) were intravenously (i.v.) treated with Nano-Folox containing 1.5 mg/kg platinum. Blood samples (˜50 μL) were collected at 1, 3, 6, 15, 30 min and 1, 4 and 12 h for ICP-MS to determine the concentration of platinum. Pharmacokinetic parameters were calculated using DAS 2.0.

In addition, — 0.05% (wt) of lipophilic carbocyanine DiD (ThermoFisher) was used to formulate the DiD-labeled Nano-Folox (1.5 mg/kg platinum). Following 8 h i.v. injection of DiD-labeled Nano-Folox, major organs and tumors were collected and analyzed using the IVIS® Kinetics Optical System, with the excitation wavelength at 640 nm and the emission wavelength at 670 nm. The concentration of platinum in major organs and tumors was also measured using ICP-MS⁸.

Therapeutic studies of Nano-Folox in orthotopic CRC model. When the luminescence intensities reached ˜0.5 to 1×10⁹ p/sec/cm²/sr, orthotopic CRC mice were i.v. injected with Nano-Folox containing 1.5 mg/kg platinum on Day 14, 17 and 20. Following 2 h injection, animals were i.p. treated with or without 50 mg/kg 5-fluorouracil (5-Fu). In addition, the FOLFOX was used as follows: mice were i.p. injected with OxP (3 mg/kg platinum), followed 2 h with FnA (90 mg/kg) and 5-Fu (50 mg/kg)^{41 42.} The luminescence intensity (p/sec/cm²/sr) was regularly measured using the IVIS® Kinetics Optical System, and the tumor growth was determined as the intensity over the initial (n=6). In separate studies, at pre-determined time points, tumors were collected for TUNEL assay (n=4)⁶¹, immunofluorescence staining (n=4)^{24 62}, flow cytometry analysis (n=4)^{30 63} and RT-PCR assay (n=4)³¹.

In order to assess in vivo toxicity, tumor-bearing mice were treated as described above except that Nano-Folox contains 3 mg/kg platinum (n=6). Body weight was recorded regularly, and the whole blood and serum of animals were collected to determine the myelosuppression (i.e., red blood cells, white blood cells, platelets and hemoglobin) and hepatic/renal functions (i.e., aspartate aminotransferase, alanine aminotransferase, creatinine and blood urea nitrogen) on Day 35. In addition, major organs were collected and analyzed using the hematoxylin and eosin (H & E) staining assay⁶⁴.

Therapeutic studies of Nano-Folox in liver metastasis model. The hemi-splenic CT26-FL3-derived liver metastasis model was established as previously described³¹. After tumor inoculation (Day 0), mice were i.p. treated with 100 μL D-luciferin (10 mg/mL; Pierce™), and the tumor burden was regularly monitored using the IVIS® Kinetics Optical System. When the luminescence intensities reached ˜0.5 to 1×10⁸ p/sec/cm²/sr, animals were i.v. injected with Nano-Folox containing 1.5 mg/kg Pt (Day 8, 12 and 16), which were followed by i.p. injection of 50 mg/kg 5-Fu at 2 h post-injection. Subsequently, animals were i.p. treated with or without anti-mouse PD-L1 mAb (α-PD-L1, Bioxcell, clone 10F.9G2, 100 μg per animal). The luminescence intensity (p/sec/cm²/sr) was regularly measured using the IVIS® Kinetics Optical System, and the tumor growth was determined as the intensity over the initial (n=5).

Statistical analysis. Results were presented as the mean±standard deviation (SD). An unpaired Student's t-test (two-tailed) was used to test the significance of differences between two mean values. A one-way ANOVA (Bonferroni's Post-Hoc test) was used to test the significance of differences in three or more groups. In all experiments, p<0.05 was considered statistically significant.

### Example 1

Preparation and physicochemical characterization of Nano-Folox. As shown in FIG. 1, in order to produce dihydrate(1,2-diaminocyclohexane)platinum(II), AgNO₃ (64.5 mg, 0.38 mmol) was added to a suspension of dichloro(1,2-diaminocyclohexane)platinum(II) (76 mg, 0.2 mmol) in 1 mL deionized water. This mixture was heated at 60° C. for 3 h and stirred in the dark at room temperature (RT) overnight. Subsequently, the mixture was centrifuged twice at 15,000 rpm for 10 min to remove the AgCl precipitate, and the supernatant was filtered through a 0.2 μm membrane. The concentration of dihydrate(1,2-diaminocyclohexane)platinum(II) was measured using inductively coupled plasma mass spectrometry (ICP-MS).

In addition, a 100 μL of 100 mM dihydrate(1,2-diaminocyclohexane)platinum(II) aqueous solution was dispersed into a 25 mL oil phase composed of cyclohexane, Triton X-100 and hexanol (75:15:10, V:V:V) to produce a water-in-oil reverse microemulsion. In addition, a microemulsion was prepared by adding 2 mL of 10 mM FnA aqueous solution into a 75 mL oil phase. 200 μL DOPA (20 mM) was then added into the FnA-contained oil phase with stirring. Following ˜10 to 20 min, two oil phases were mixed and stirred for ˜30 to 45 min. Subsequently, 100 mL ethanol were added for ˜15 min, and the mixture was centrifuged at 12,000 g for 20 min to collect the precipitation (FIG. 1A). The precipitation was thoroughly washed with ethanol twice, and was re-dispersed in chloroform.

In order to produce Nano-Folox, 1 mg core, 10 μL of 20 mM DOTAP, 10 μL of 20 mM cholesterol and 5 μL of 20 mM DSPE-PEG/DSPE-PEG-AEAA (molar ratio=4:1) were dissolved in chloroform. After the chloroform evaporation, the lipid film was rehydrated in deionized water to form Nano-Folox.

The measurements of particle size and zeta potential of Nano-Folox were performed using the Malvern Nano-ZS (Malvern Instruments, UK)⁵⁹. The morphology of nanoprecipitates and Nano-Folox was analyzed using transmission electron microscopy (TEM). Briefly, 5 μL Nano-Folox were added on 400-mesh carbon-filmed copper grids (Agar Scientific) for 2 min. The samples were stained with 2% (w/w) uranyl acetate before the analysis using the JEM1230 (JEOL) TEM. Alternatively, the morphology of the core was analyzed without the negative staining.

In line with the platinum content of a different drug⁸, a suspension of Nano-Folox containing 250 μg platinum in 0.01 M PBS (pH=5.5 and 7.4) was incubated at 37° C. with slight shaking. At different time points, the samples were centrifuged at 15,000 rpm for 30 min and the platinum release into the supernatant was measured using ICP-MS.

Preparation and physicochemical characterization of Nano-Folox. OxP, the third-generation platinum-based drug with a 1,2-diaminocyclohexane (DACH) ring and an oxalate group, are primarily applied in the treatment of CRC at advanced stages and when hepatic metastases grow⁹. It has been proposed that OxP undergo a series of non-enzymatic biotransformation in physiological situations^{10, 11}. The oxalate ligand of OxP is spontaneously displaced by nucleophiles (e.g., chloride), resulting in formation of dichloro(1,2-diaminocyclohexane)platinum(II) (Pt(DACH)Cl₂, an intermediate derivate)¹². When the chloride moieties are chemically substituted with aqua ligands, Pt(DACH)Cl₂ is converted into [Pt(DACH)(H₂O)₂]²⁺ (the activate form of OxP)¹³. In addition to the aforementioned biotransformation, it is also likely that the carboxylate ligands of OxP is directly replaced by aqua ligands forming [Pt(DACH)(H₂O)₂]^{2+ 14}. Consequently, [Pt(DACH)(H₂O)₂]²⁺ reacts with DNA to generate Pt-DNA adducts, which inhibit the replication and transcription of DNA, resulting in DNA strand break and cellular apoptosis¹⁵.

### Example 2. Synthesis of Dihydrate(1,2-diaminocyclohexane)platinum(II)

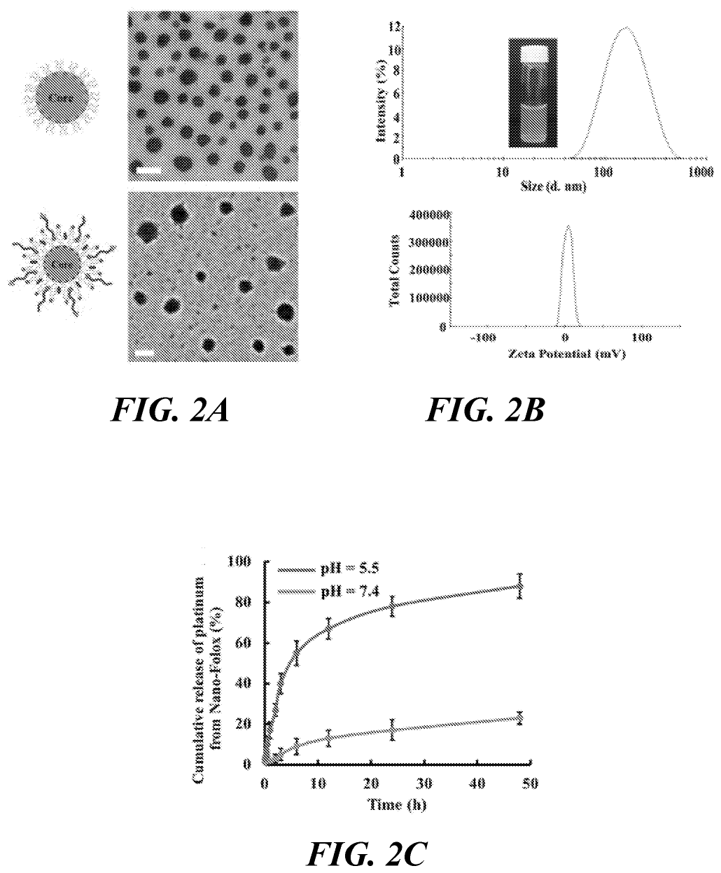

In this study, as shown in FIG. 1A, [Pt(DACH)(H₂O)₂]²⁺ was synthesized by a reaction between Pt(DACH)Cl₂ and AgNO₃ where the chloride moieties were chemically substituted with aqua ligands^{6 7 8}. A Pt(DACH).FnA precipitate was subsequently formed by conjugation of [Pt(DACH)(H₂O)₂]²⁺ and FnA at an equimolar ratio in a water-in-oil reverse microemulsion. The formation of the precipitate (C₂₆H₃₅N₉O₇Pt) was supported by (FIG. 1C) mass spectrometry (predicted exact mass: 780.23, observed m/z 780.96). An excess of FnA was used to maximize the precipitation (the yield of platinum was ˜55% as determined by ICP-MS). The DOPA, which can strongly bind on the surface of platinum cation²⁰, was used to stabilize the precipitate and facilitate control over the particle size (˜100 nm) (FIG. 2A). The stabilized nanoprecipitates were poorly soluble in water; therefore, the outer surface of precipitation core was coated with DOTAP, cholesterol, DSPE-PEG and DSPE-PEG-AEAA, in order to achieve a targeted formulation in aqueous solutions (namely Nano-Folox, the drug loading efficiency 70 wt %) (FIG. 1A).

Nano-Folox demonstrated nanoscale particle size (˜120 nm, polydispersity index=0.3) and nearly neutral zeta potential (˜5 mV) (FIG. 2B). The increased particle size observed from Nano-Folox suggest the attachment of DOTAP, cholesterol, DSPE-PEG and DSPE-PEG-AEAA onto the nanoprecipitates. In addition, a thin “halo-like” layer was observed on the surface of Nano-Folox (FIG. 2A), which was different from the morphology of the stabilized nanoprecipitates, further indicating the successful coating.

### Example 3. Release Kinetics

In order to achieve the safe and efficient delivery of chemotherapeutics, the drug carriers are required to avoid burst release in the systemic circulation, but can provide drug release inside cancer cells. As shown in FIG. 2C, ˜20% of Pt was released from Nano-Folox at 48 h in neutral PBS; on the contrary, the release rate (>90%) was significantly increased when a pH was changed from 7.4 to 5.5. We have previously demonstrated that LPI is stable in PBS and can release the cargo in the presence of lipase or surfactant⁸, the release profile of which is reminiscent of that observed from Doxil® (the crystalline doxorubicin is encapsulated inside)²¹. Due to the coating structure of Nano-Folox similar to LPI, these results suggest that the stability of Nano-Folox may be maintained during the blood circulation, and when Nano-Folox arrives inside cancer cells, the lipid layer may be lysed resulting in release of platinum drug from late endosomes in which the lipase exists and the pH becomes ˜5-6.

### Example 4. In Vitro Characterization of Nano-Folox

The CT26-FL3 as the most highly metastatic subtype of CT26 (a mouse colon carcinoma cell line) causes primary tumor and hepatic metastasis when implanted at the cecum wall of mice²². In this study, CT26-FL3 cells were used for in vitro characterization of Nano-Folox as discussed below.

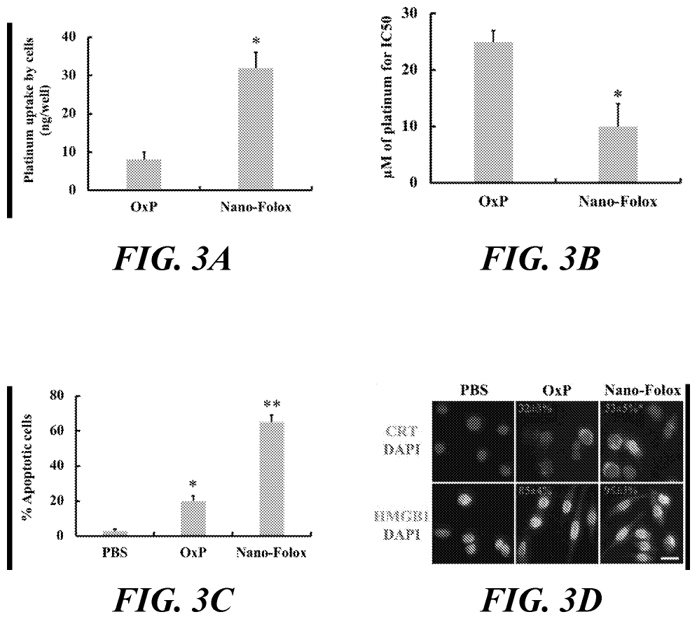

The aminoethyl anisamide (AEAA) targeting ligand has been exploited in our laboratory to specifically deliver drugs/genes into sigma receptor overexpressing cancer cells and characterized in murine models of melanoma^{23 24} breast cancer^{25 26}, pancreatic cancer^{27 28}, bladder cancer²⁹, and CRC^{30 31}. Previously, the AEAA-mediated targeting effect has been confirmed by transfection of plasmid DNA in CT26-FL3 cells^{30 31} In this study, Nano-Folox achieved significantly higher cellular uptake (up to 4 folds) of platinum in comparison with OxP (FIG. 3A), implying that delivery of Nano-Folox is also enhanced likely due to the AEAA targeting effect (see biodistribution as discussed below).

Following efficient cellular uptake, the Pt(DACH).FnA precipitate is ready for release from Nano-Folox. The precipitate possesses the carboxylate ligands, which are similar to those of OxP (FIG. 1B). Therefore, it suggests that the ligand-exchange reaction involved in the conversion of OxP into [Pt(DACH)(H₂O)₂]²⁺ also occurs in the Pt(DACH).FnA precipitate. As shown in FIG. 1B, the precipitate is likely dissociated in the present of chloride leading to the formation of Pt(DACH)Cl₂ and FnA. Following the substitution of aqua ligands, Pt(DACH)Cl₂ is further converted into [Pt(DACH)(H₂O)₂]²⁺ (FIG. 1B). In addition, [Pt(DACH)(H₂O)₂]²⁺ may also be directly generated when the carboxylate ligands of Pt(DACH).FnA is displaced by aqua ligands. In fact, the structure of Pt(DACH).FnA precipitate is also reminiscent of that observed in carboplatin, the second-generation platinum-based drug with a bidentate dicarboxylate chelate leaving group³². Carboplatin, once inside cells, undergoes a stepwise aquation to form [Pt(NH₃)₂(H₂O)₂]^{2+ 33}, which subsequently lead to platinum-DNA adduct structures. The metabolic activity of Pt(DACH).FnA will be investigated in the future to confirm these hypotheses.

### Example 5. Anti-Proliferation Property of Nano-Folox

As shown in FIG. 3B, Nano-Folox significantly slowed down the proliferation of CT26-FL3 cells (p<0.05; IC50≈10 μM Pt, 24 h incubation), whereas OxP achieved less anti-proliferative potent (IC50 24 μM Pt, 24 h incubation) (OxP was chosen as a control due to the insolubility and ineffective suspension of nanoprecipitates in aqueous solutions). FnA is generally considered non-toxic but can enhance the anti-tumor efficacy of 5-Fu. Indeed, the proliferation of CT26-FL3 cells was not inhibited by FnA alone (data not shown), indicating that the anticancer effect achieved by Nano-Folox is mainly resulted from the OxP derivative. In addition, Nano-Folox induced a significant level of apoptosis in CT26-FL3 cells (p<0.05, 24 h incubation) compared to OxP (FIG. 3C), indicating that the anti-proliferative effect achieved by Nano-Folox is, at least, in part due to the apoptosis. These results further confirmed that the Pt(DACH).FnA can be metabolized inside CT26-FL3 cells to achieve the anticancer activity.

Recently, it has been reported that a form of apoptosis termed immunogenic cell death (ICD, also known as immunogenic apoptosis) can be induced by a group of chemotherapeutic drugs (e.g., anthracyclines and OxP) and by physical treatments (e.g., ionizing irradiation and photodynamic therapy)³⁴. ICD is described to cause cancer cell death in a manner that induces the immune response to activate T lymphocytes for recognizing tumor-specific antigens^{35 36}. ICD inducers mediate the activation of damage-associated molecular patterns (DAMPS) molecules, mainly including the calreticulin (CRT) exposure, adenosine triphosphate (ATP) secretion, and high mobility group B1 (HMGB1) release³⁷. In this study, the potential of Nano-Folox for ICD-induced cancer cell immunogenicity was assessed in terms of CRT exposure and HMGB1 release³⁸ (FIG. 3D). Results demonstrate that the exposure of CRT on the cell surface was significantly induced by Nano-Folox (p<0.05; 10 μM Pt, 2 h incubation) compared to OxP at the same conditions. In addition, Nano-Folox (10 μM Pt, 8 h incubation) showed a slight increase (p>0.05) in the release of HMGB1 from the nucleus into the cytoplasm compared to OxP at the same conditions (FIG. 3D). In contrast, neither CRT exposure nor HMGB1 release was evident with the PBS control group. These results indicate the potential of Nano-Folox as nanoparticulate ICD inducer delivery system for CRC.

### Example 6. Pharmacokinetics and Biodistribution of Nano-Folox

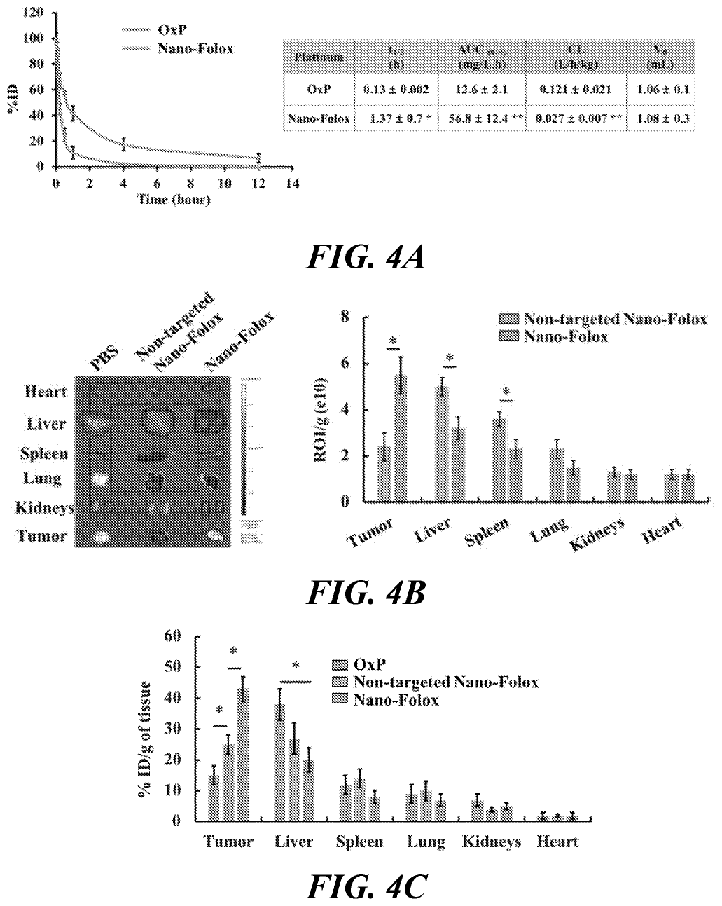

The pharmacokinetics of Nano-Folox was investigated using an orthotopic CRC mouse model. Following a single intravenous (i.v.) injection of OxP and Nano-Folox, the plasma concentrations of platinum versus time (n=4 mice per group) are shown in FIG. 4A. The concentrations of platinum in the plasma for OxP decreased rapidly, and only a residual level was detected less than 4 h post injection. By contrast, the platinum in Nano-Folox was more slowly removed from the plasma, over 12 h post injection (FIG. 4A).

The pharmacokinetic profiles were analyzed by fitting to a one-compartmental model (FIG. 4B). Nano-Folox achieved a significantly higher value of area under the curve (AUC) than that of OxP (p<0.05). Nano-Folox also significantly reduced clearance values (CL) compared to OxP (p<0.05). Correspondingly, a significantly longer half-life (t_{1/2}) was recorded by Nano-Folox (˜80 min) than OxP (˜8 min) (FIG. 4B). These pharmacokinetic parameters indicate that Nano-Folox led to a ˜10-fold increase in systemic circulation of platinum relative to OxP.

The tissue distribution of Nano-Folox was also evaluated using orthotopic CRC mice. Eighth after i.v. injection of a single dose containing DiD-labeled NPs (n=4), major tissues and tumors were collected and imaged using the IVIS® Kinetics Optical System (FIG. 4C). Results show that Nano-Folox with the AEAA targeting ligand achieved a significantly higher retention in tumor than non-targeted counterpart (p<0.05); in contrast, a significantly less accumulation in liver and spleen was found in AEAA-targeted Nano-Folox (p<0.05). In addition, the tissue distribution of platinum was measured ex vivo using ICP-MS 8 h after i.v. administration of OxP and Nano-Folox with/without AEAA (n=4) (FIG. 4D). Similar to results obtained using the imaging system, AEAA-targeted Nano-Folox achieved a significantly higher tumor accumulation of platinum (˜45% ID/g) than non-targeted counterpart (˜25% ID/g) and OxP (˜15% ID/g). In contrast, the liver uptake of platinum for OxP (˜35% ID/g) was significantly higher than those of Nano-Folox with/without AEAA (˜20% ID/g and ˜25% ID/g, respectively) (FIG. 4D). Therefore, these results indicate that the addition of AEAA targeting ligand enhanced the tumor retention and reduced non-specific tissue accumulation.

It has been reported that when OxP is i.v. administrated, the platinum is irreversibly absorbed onto plasma proteins and erythrocytes¹¹, which significantly lessen the therapeutic efficacy of OxP. As a result, the bound Pt tends to the rapid systemic elimination via the renal clearance³⁹. Generally, CRC patients are given FOLFOX with a number of cycles in order to achieve therapeutic outcome. For instance, 12 cycles of FOLFOX were required to achieve an increased overall survival of patients with CRC at stages II/III in a trial of the Multicenter International Study of Oxaliplatin/5-Fluorouracil/Leucovorin in the Adjuvant Treatment of Colon Cancer (MOSAIC)⁴⁰. However, the side effects or toxicities are often caused by such intensive treatment, and patients also suffer from high expenses and time-consuming treatment schedule (e.g., total cycles >24 weeks).

As shown in FIGS. 4A and 4B, Nano-Folox significantly enhanced the blood circulation of platinum relative to OxP, indicating the potential for a reduced number of treatment cycles achieving the same therapeutic benefit. In addition, due to the enhanced tumor accumulation achieved by AEAA-mediated targeting effect (FIGS. 4C and 4D), Nano-Folox potentially provides a low-dosage strategy that is sufficient for treating patients. In summary, Nano-Folox demonstrates significant potential to overcome the limitations associated with FOLFOX. The therapeutic potential for a combination of Nano-Folox with 5-Fu as a novel FOLFOX regimen was then investigated.

### Example 7. The Combination of Nano-Folox and 5-Fu Achieved an Enhanced Chemo-Immunotherapy in Orthotopic CRC Mice

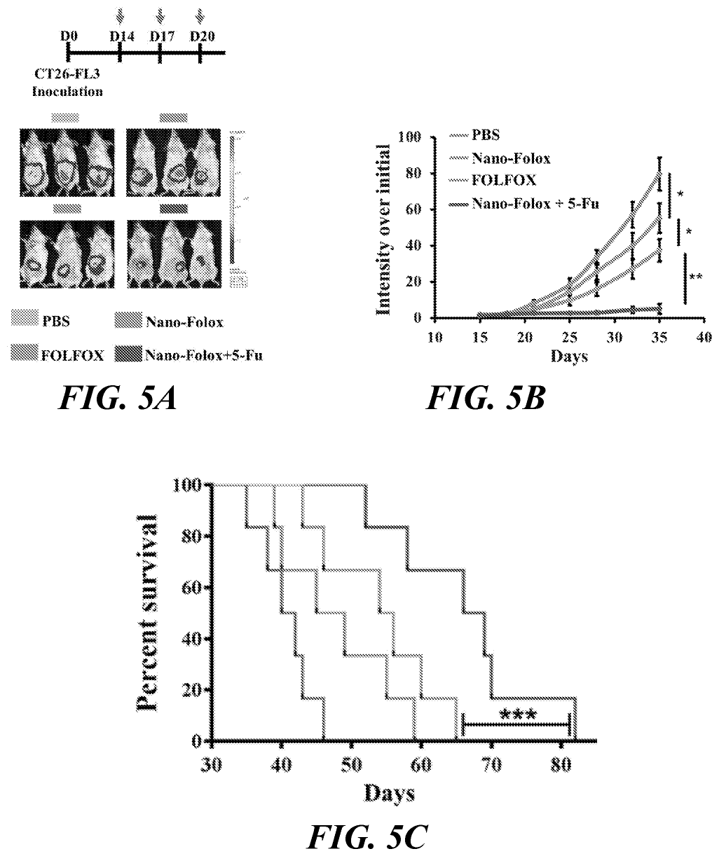

The therapeutic efficacy of Nano-Folox in the orthotopic mouse model was assessed following i.v. injections of PBS, Nano-Folox, the FOLFOX, and the combination of Nano-Folox and 5-Fu (n=6, see the treatment scheme in FIG. 5A). As CT26-FL3 cells stably express the firefly luciferase gene that catalyzes the oxidation of luciferin to generate bioluminescence³⁰, the development of tumor in situ can be monitored by using the IVIS® Kinetics Optical System (FIG. 5A). The therapeutic efficacy of Nano-Folox was dependent on the administration dose (0.5 to 5 mg/kg Pt, data not shown), and Nano-Folox containing 1.5 mg/kg platinum onwards could significantly (p<0.05) slowed down the tumor growth relative to the PBS control group (FIG. 5B). When a combination of Nano-Folox (1.5 mg/kg platinum) and 5-Fu (50 mg/kg) was given to tumor-bearing animals, the anti-tumor efficacy was significantly (p<0.01) higher than Nano-Folox alone and the FOLFOX (3 mg/kg platinum, 90 mg/kg FnA and 50 mg/kg 5-Fu; this drug-dosing schedule was based on studies published in 41 42) (FIG. 5B). Consequently, the combination of Nano-Folox and 5-Fu significantly (p<0.001) improved the survival of diseased mice relative to the other groups (FIG. 5C). Thus, Nano-Folox with 5-Fu showed improved therapeutic effect at a lower dose of platinum as compared with FOLFOX.

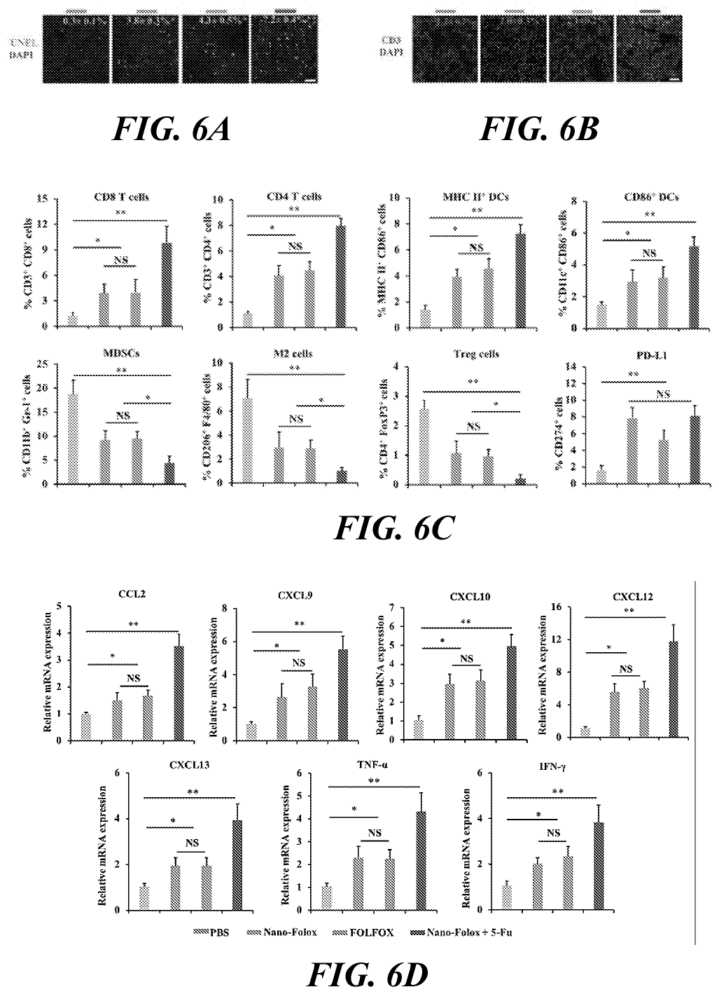

The anti-tumor mechanisms of Nano-Folox were also investigated using orthotopic CRC mice (FIG. 6). Results show that Nano-Folox significantly (p<0.05) induced cell apoptosis relative to PBS control group (FIG. 6A). It implies that the Pt(DACH).FnA precipitate was successfully dissociated into [Pt(DACH)(H₂O)₂]²⁺ and FnA inside cells, in which the [Pt(DACH)(H₂O)₂]²⁺ forms the DNA-adducts resulting in the apoptosis. Furthermore, an improved apoptotic effect was achieved by the combination of Nano-Folox and 5-Fu (FIG. 6A), which is likely that the anti-tumor efficacy of 5-Fu is enhanced by FnA released from Nano-Folox. As a result, the combination of Nano-Folox and 5-Fu significantly (p<0.05) enhanced cell apoptosis relative to Nano-Folox alone and the FOLFOX (FIG. 6A).

In addition, flow cytometry results demonstrate that the level of CD8⁺ cytotoxic T cells and CD4⁺ helper T cells was significantly increased inside tumors following the combined treatment (FIG. 6C), which is accompanied with the enhancement of T lymphocyte recruitment (FIG. 6B). Also, MHC It and CD86⁺ dendritic cells (DCs) were significantly activated by the combinatorial approach (FIG. 6C). Corresponding to these immune stimulatory effects, the amount of suppressive immune cells (e.g., myeloid-derived suppressor cells (MDSCs), regulatory T cells (Tregs) and tumor-associated macrophages (M2)) was significantly decreased after the combination therapy (FIG. 6C). In addition, the pro-inflammatory cytokines (e.g., CCL2, CXCL12 and CXCL13) were significantly (p<0.05) activated in the tumor treated with Nano-Folox/5-Fu relative to Nano-Folox alone and the FOLFOX (FIG. 6D). The cytokines CXCL9 and CXCL10, which are in favorable of T cell infiltration, were also increasingly induced for the combination strategy (FIG. 6D). Th1-type cytokines IFN-γ and TNF-α were also significantly elevated accordingly (FIG. 6D). These results suggest that the combination of Nano-Folox and 5-Fu can effectively trigger the ICD effect in tumors, which may release cancer cell associated antigens and mediate DC maturation with cross-priming capacity to CD8⁺ cytotoxic T cells. Consequently, the activated CD8⁺ cytotoxic T cells are recruited to induce the perforin/granzyme cell death pathway, achieving the inhibition of tumor growth⁴³.

### Example 8. In Vivo Toxicity Studies

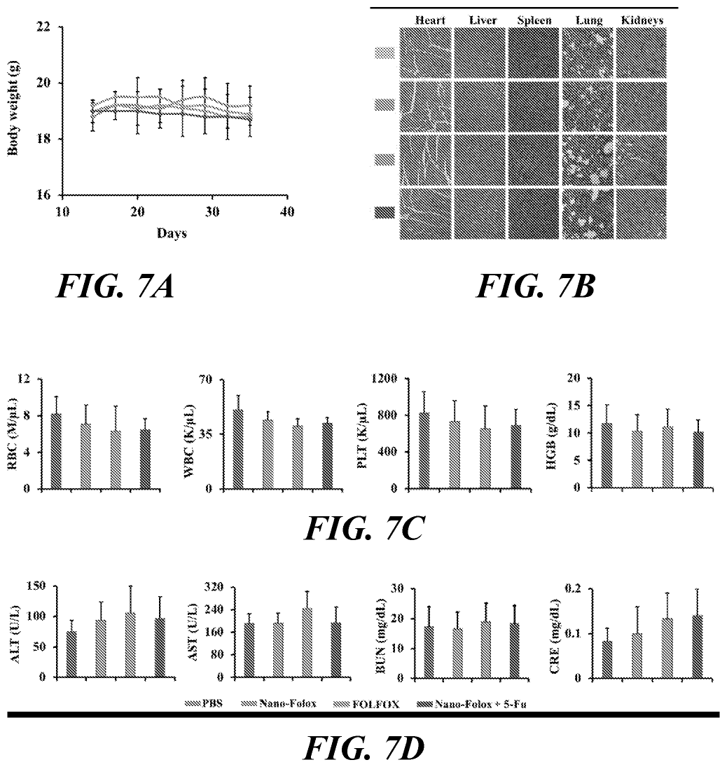

Orthotopic CRC mice were given i.v. injections of PBS, Nano-Folox, the FOLFOX, and the combination of Nano-Folox and 5-Fu (n=4 mice per group) (FIG. 7). Monitoring of animal body weight showed no significant decrease in therapy groups over a 3-week period relative to PBS control group (FIG. 7A). Major tissues including the heart, liver, spleen, lung and kidneys were analyzed using H & E staining assay. No significant histological damage between samples from animals treated with PBS and therapy groups was detected (FIG. 7B). In addition, the whole blood cellular components (FIG. 7C) and the serum liver/kidney function markers (FIG. 7D) were analyzed to further assess systemic toxicity. The results show no significant hematological toxicity following the treatments in comparison with PBS control group (FIG. 7C). In addition, the level of aspartate aminotransferase (AST), alanine aminotransferase (ALT), creatinine (CRE) and blood urea nitrogen (BUN) in the serum was not significantly altered by the therapeutic groups (FIG. 7D). Therefore, these toxicology studies indicate no significant signs of systemic toxicity for Nano-Folox and the combination with 5-Fu.

### Example 9. The Anti-PD-L1 Monoclonal Antibody Synergized with the Combination of Nano-Folox and 5-Fu for Decreased Liver Metastasis

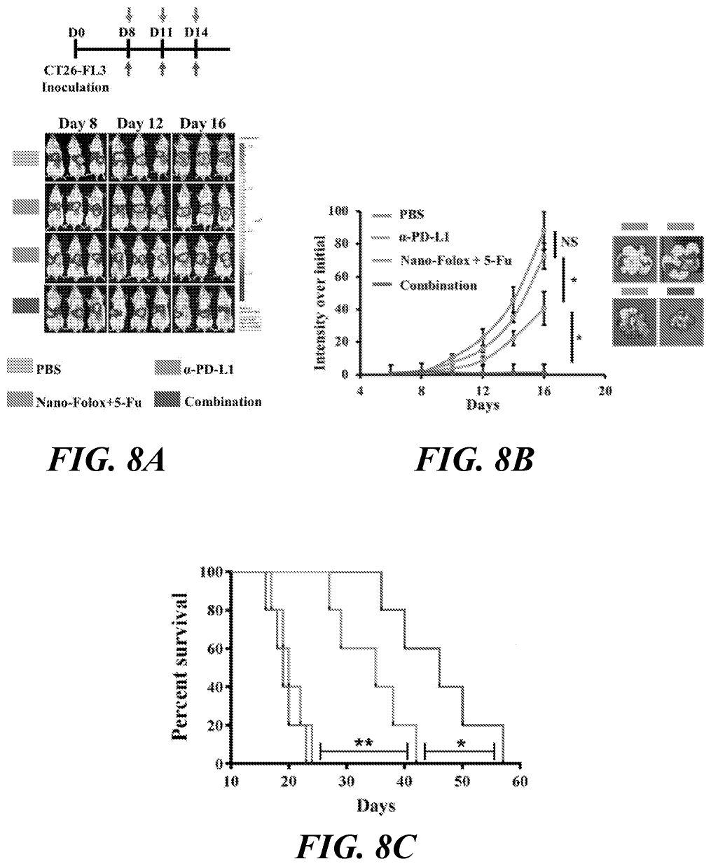

As shown in FIG. 6C, although the combination of Nano-Folox and 5-Fu induced an effective anti-tumor immunological response, the increased level of programmed death ligand 1 (PD-L1) protein was detected in tumor tissues. PD-L1 is known to bind with the programmed death 1 (PD-1), which activate the PD-1/PD-L1 signaling pathway, promoting cancer cell immune evasion⁴⁴. Recently, the anti-PD-L1 antibody (e.g., pembrolizumab and nivolumab) has been used for the treatment of microsatellite instability (MSI)-high or mismatch repair (MMR)-deficient CRC^{45 46}. In this study, the potential of anti-PD-L1 monoclonal antibody (mAb) to enhance the therapeutic efficacy of Nano-Folox/5-Fu was assessed using mice with experimental liver metastasis (FIG. 8A). The diseased model is established by hemi-splenic inoculation of CT26-FL3 cells into the liver through the portal venous system⁴⁷, which highly reproduce the metastatic pattern of human CRC at advanced stages. As shown in FIG. 8B, no significant anti-metastasis effect was achieved by anti-PD-L1 mAb relative to PBS control group, which is similar to results obtained in previous studies^{30 31} It may be explained that CT26 is an MMR-proficient CRC cell line^{48 49}, and the anti-PD-L1 mAb is less capable for providing therapeutic efficacy in MMR-proficient tumor models^{30 31} In contrast, the liver metastasis was significantly (p<0.05) reduced by Nano-Folox/5-Fu compared to anti-PD-L1 mAb and PBS (FIG. 8B). Furthermore, the combination of Nano-Folox/5-Fu and anti-PD-L1 mAb demonstrated a synergistic therapeutic effect relative to either of treatment strategies (FIG. 8B), significantly (p<0.05) prolonging the survival of animals (FIG. 8C). Therefore, these results indicate the therapeutic potential of this combination strategy in the treatment of metastatic CRC.

### Materials and Methods for Examples 10-15

### Materials

5-Fluoro-2′-deoxyuridine 5′-monophosphate (FdUMP), 2′-deoxyuridine 5′-monophosphate (dUMP), IGEPAL® CO-520, cyclohexane, Triton X-100, CaCl₂), (NH₄)₂HPO₄, cholesterol, folinic acid (FnA) and 5-Fluorouracil (5-Fu) were obtained from Sigma-Aldrich. Oxaliplatin (OxP) was obtained from Selleckchem. 1,2-dioleoyl-sn-glycero-3-phosphate (DOPA) and 1,2-dioleoyl-3-trimethylammonium-propane (DOTAP) were purchased from Avanti Polar Lipids. N-(Carbonyl-methoxypolyethyleneglycol 2000)-1,2-di stearoyl-sn-glycero-3-phosphoethanolamine (SUNBRIGHT® DSPE-020CN; DSPE-PEG) was obtained from NOF CORP. DSPE-PEG-AEAA was synthesized as previously demonstrated in our laboratory.¹²³

### Preparation and Characterization of Nanoformulations

Nano-FdUMP was prepared as previously described with modifications.^{84,85} Briefly, 1 mL of FdUMP solution (1 mg/mL) was added into 2 mL of CaCl₂ solution (2.5 M), and this mixture was added into 80 mL oil phase composed of IGEPAL® CO-520 and cyclohexane (30:70, V:V) for generation of water-in-oil reverse microemulsion. Another microemulsion (80 mL) was prepared by adding 2 mL of (NH₄)₂HPO₄ solution (50 mM) and 1 mL of DOPA solution (20 mM in chloroform). Two microemulsions were thoroughly stirred for ˜15 to 20 min. After this, 160 mL of ethanol were added for ˜15 to 20 min with stirring, which was followed by centrifugation for 20 min at 10,000 g for collection of nanoprecipitates. Nanoprecipitates were washed using ethanol, dried using nitrogen, and stored in chloroform.

The optimal ratio between nanoprecipitates and outer leaflet lipids for Nano-FdUMP was as follows: 1,500 μg of nanoprecipitates, 30 μL of DOTAP (25 mM), 30 μL cholesterol (25 mM) and 20 μL DSPE-PEG/DSPE-PEG-AEAA (20 mM, molar ratio=5:1) in 2 mL of chloroform. This theoretically achieved ˜3.5 mol % of AEAA on the outer lipid surface per formulation. Following evaporation of chloroform, the lipid film was resuspended using aqueous solution to form Nano-FdUMP. The encapsulation efficiency and loading capacity were assessed using HPLC (Shimadzu, Japan) (C18 column, UV at 250 nm, mobile phase=water and methanol, 85:15). Nano-dUMP and non-targeted Nano-FdUMP were prepared as mentioned above except the use of dUMP and the lack of DSPE-PEG-AEAA, respectively. Nano-Folox was prepared as previously described.⁷¹

The hydrodynamic diameter and zeta potential of NPs were measured using Malvern Nano-ZS. The morphology of NPs was observed using the JEM1230 (JEOL) transmission electron microscope (TEM) as described previously.¹¹⁶ In addition, a solution of NPs with 200 μg of FdUMP was incubated at 37° C. in 0.01 M PBS (pH=5.5 and 7.4) with shaking. Samples were obtained at different time points for centrifugation at 10,000 g for ˜30 min. The concentration of free FdUMP within supernatants (dissociated from nanoprecipitates) was determined using HPLC.

### Cell Culture

CT26 (mouse CRC cell line), Hepa1-6 (mouse HCC cell line), 4T1 (mouse breast cancer cell line) and B16 (mouse melanoma cell line) cells were cultured using DMEM (Gibco) with 10% bovine calf serum (Hyclone), and 1% antibiotic-antimycotic (Gibco). CT26-FL3 (a subtype of CT26, it is engineered to stably express luciferase) and Hepa1-6-Luc (it is engineered to stably express luciferase) cells,^{71, 124} were cultured using the aforementioned growth medium with 1 μg/mL puromycin (ThermoFisher). Cells were maintained at 37° C. with 5% CO₂ and 95% relative humidity.

### In Vitro Studies

MTT assay was applied to determine in vitro cytotoxicity. CT26 and Hepa1-6 cells (1×10⁴/well) were cultured within 96-well plates, respectively. Following one day incubation, 5-Fu, Nano-dUMP and Nano-FdUMP were added to cells for 24 h. Cells were then added with MTT reagent at 37° C. for ˜4 h before measurement at 570 nm. IC₅₀ was calculated using the GraphPad Prism software.

CT26 and Hepa1-6 cells (5×10⁴/well) were placed into 24-well plates, respectively. After one day incubation, cells were treated with or without N-acetylcysteine (NAC; 5 mM) for 4 h. Cells were replaced with fresh growth medium and added with 5-Fu, Nano-dUMP and Nano-FdUMP (all at 15 μM) for 24 h. Subsequently, apoptotic cells were detected using Annexin V-FITC/propidium iodide assay (Promega) and measured by the Becton Dickinson FACSCalibur. In a separate experiment, the ROS level in cells was detected using 2′,7′-dichlorodihydrofluorescein diacetate-based Reactive Oxygen Species Assay Kit (YIASEN Biotech) by microplate reader (488 nm/525 nm).

CRT and HMGB1 were detected using immunofluorescence staining as previously described.^{7,60} CT26 and Hepa1-6 cells (60,000 per well) were cultured in 8-well chamber slides (ThermoFisher). Following one day incubation, cells were treated with or without NAC (5 mM) for 4 h. Cells were then replaced with fresh growth medium and treated with either Nano-FdUMP (15 μM), Nano-Folox (5 μM), or both (Nano-Folox was first added, and FdUMP was added at 2 h later; this sequential administration was same for in vitro studies unless mentioned otherwise). Two h post treatment, cells were incubated with 0.25% paraformaldehyde (PFA). Following 5 min incubation, cells were washed with PBS, which were followed by application of anti-CRT antibody (ab2907, Abcam, 1:500) for 1 h. After PBS washes, FITC-conjugated secondary antibodies (ab150077, Abcam) were added into cells for 30 min. Subsequently, cells were treated by 4% PFA for 20 min and stained using DAPI (ThermoFisher) for confocal imaging (LSM-710, Zeiss). In a separate experiment, 8 h post treatment of either Nano-FdUMP (15 μM), Nano-Folox (5 μM), or both, cells were treated with 4% PFA for 30 min and 0.1% Triton X-100 for 10 min. Following PBS washes, cells were incubated with 1% bovine serum albumin (BSA) for 30 min. Cells were washed with PBS and added with anti-HMGB1 antibody (ab18256, Abcam) for 1 h. After PBS washes, FITC-conjugated secondary antibodies were added into cells for 30 min for confocal imaging.

In order to measure extracellular ATP, CT26 and Hepa1-6 cells were placed into 24-well plates at a density of 60,000 cells per well. After one day incubation, cells were treated with or without NAC (5 mM) for 4 h. Cells were replaced with fresh growth medium and added with either Nano-FdUMP (15 μM), Nano-Folox (5 μM), or both for 24 h. Subsequently, extracellular ATP was detected using ENLITEN® ATP Assay System Bioluminescence Detection Kit.

### In Vivo Toxicity, Pharmacokinetics and Biodistribution

Six-week old female BALB/C and male C57BL/6 mice were purchased from Charles River Laboratories. The procedures used in this study were approved by Institutional Animal Care and Use Committee of University of North Carolina at Chapel Hill and by the Animal Ethics Committee of Jilin University.

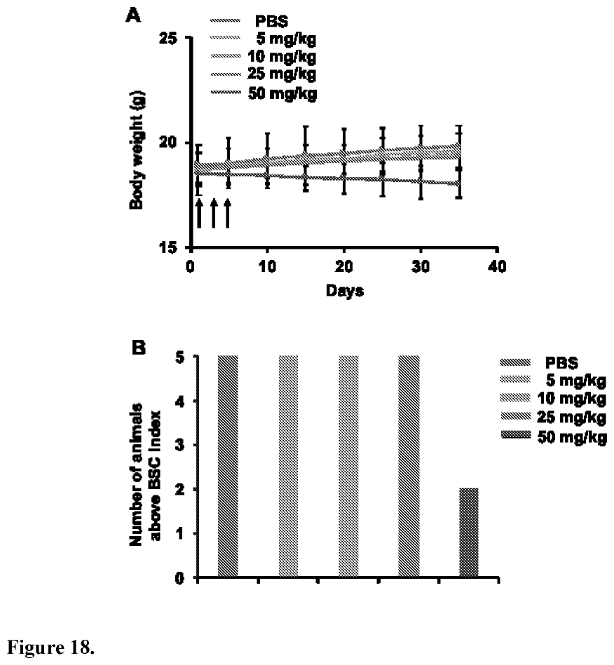

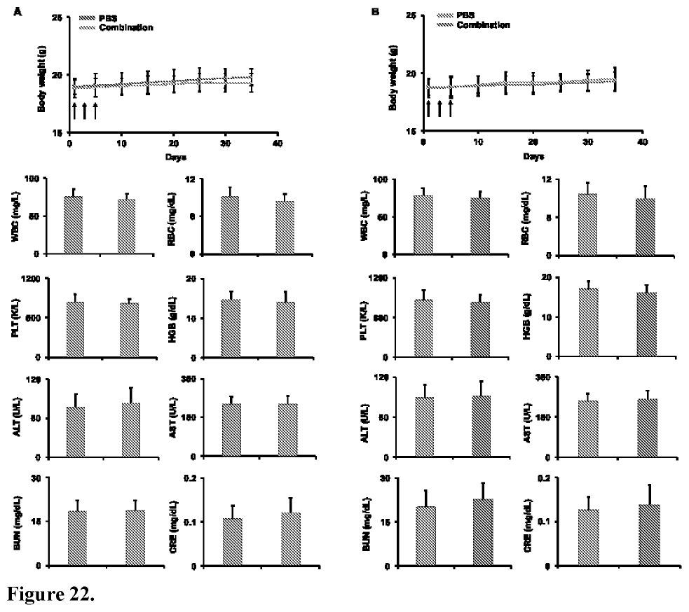

Healthy mice were treated with nanoformulations as described in FIGS. 18 and 22 (n=5). Body weight was regularly recorded, and the whole blood and the serum of animals were obtained on Day 35 to analyze myelosuppression and hepatic/renal functions.

The orthotopic CRC mouse model was achieved as previously described.⁷¹ Briefly, BALB/C mice were anesthetized by 2.5% isoflurane, and the cecum wall was injected with ˜1×10⁶ CT26-FL3 cells. In addition, the orthotopic HCC mice were established as previously described.⁶¹ Briefly, C57BL/6 mice were anesthetized by 2.5% isoflurane, and the liver was injected with ˜1×10⁶ Hepa1-6-Luc cells. Following tumor inoculation (Day 0), animals were intraperitoneally (i.p.) injected with 100 μL luciferin (10 mg/mL; Pierce™), and tumor growth was measured using IVIS® Kinetics Optical System (Perkin Elmer). When tumor growth was reached at ˜0.5 to 1×10⁹ p/sec/cm²/sr, pharmacokinetics and tissue distribution studies were investigated as follows: 1) 5-Fu (10 mg/kg) or Nano-FdUMP containing 10 mg/kg of fluorine drug were i.v. administrated, and the blood (˜50 μL) was collected at 1, 5, 10, and 15 min, and 0.5, 1, 4, 8 and 12 h (n=4). As previously described,⁶² plasma samples were extracted with ethyl acetate, dried with nitrogen, and reconstituted in the mobile phase (water/methanol, 85:15). The concentration was assessed using HPLC (Shimadzu, Japan) (C18 column, UV at 265 nm for 5-Fu and UV at 250 nm for FdUMP). Half-life was evaluated using DAS 2.0 software. In separate studies, ˜0.05 wt % of DiD (ThermoFisher) was formulated into Nano-FdUMP or non-targeted counterpart (10 mg/kg of fluorine drug). Twelve h post i.v. administration, distribution of DiD-labeled nanoformulations into tissues and tumors was detected (640 nm/670 nm) using IVIS® Kinetics Optical System (n=4).

### Synergistic Efficacy of Nano-FdUMP and Nano-Folox in Orthotopic CRC and HCC Mouse Models

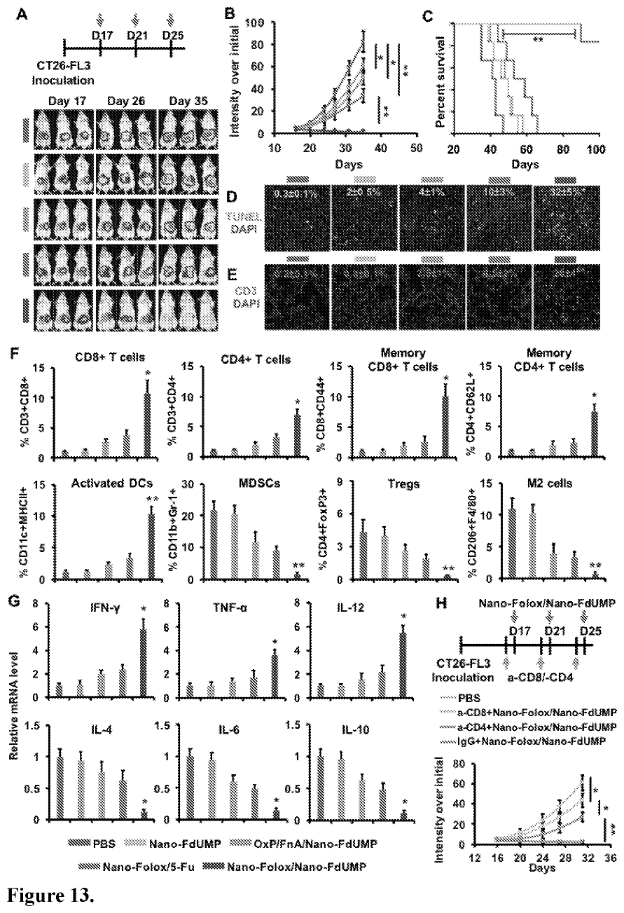

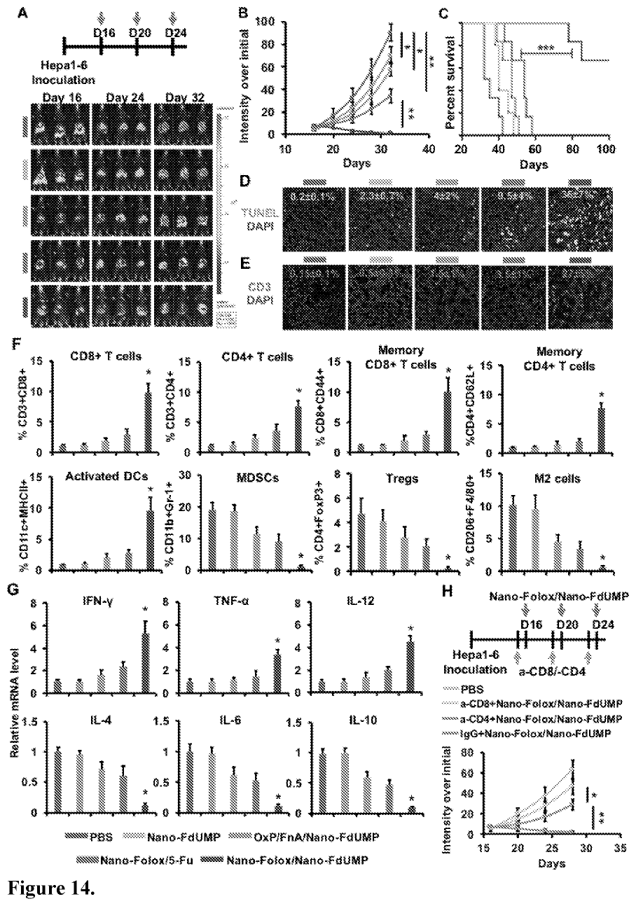

When tumor growth was reached at ˜0.5 to 1×10⁹ p/sec/cm²/sr, tumor-bearing mice were injected with either OxP/FnA (1.5 mg/kg and 4.5 mg/kg, i.v.) or Nano-Folox containing 1.5 mg/kg of platinum drug (i.v.; it contained ˜4.5 mg/kg of FnA) as described in FIGS. 13 and 14. Eight h post injection (t_{1/2} of Nano-Folox 1.4 h), tumor-bearing mice were treated with either 5-Fu (10 mg/kg; i.v.) or Nano-FdUMP containing 10 mg/kg of fluorine drug (i.v.). Tumor growth was observed using the IVIS® Kinetics Optical System (n=6).

In separate experiments, 3 days after two injections (time point to analyze chemotherapeutic and immunotherapeutic effects was generally chosen within one week following treatment to ensure reliable analyses) 74, 91, 127, 128, tumors were obtained on Day 24 (CRC) and Day 23 (HCC) for following assays: 1) TUNEL assay.^{71, 124} It was performed using the DeadEnd™ Fluorometric TUNEL System (Promega) (n=4). DNA fragments (FITC) and nuclei (DAPI) were detected by confocal microscopy; 2) Immunofluorescence assay.^{71, 124} Tumors were added with 4% PFA for ˜24 h and conducted on paraffin-embedded slices (n=4). Slices were treated with de-paraffinization, retrieval of antigen, permeabilization, and blocking of 1% BSA.

Antibodies with fluorophores were added to slides overnight at 4° C. (see Supplementary Table 1), and analyzed using confocal microscopy. 3) Flow cytometry.^{71, 124} Tumors (n=4) were treated using collagenase A (1 mg/mL; Sigma) and DNAse (200 μg/mL; Invitrogen) for 30 min at 37° C. to produce single cells. After lysis of erythrocytes with ACK buffer (Gibco), cells were treated by fluorophore-labeled antibodies (see Supplementary Table 1), fixed using 4% PFA, and assessed using the Becton Dickinson LSR II. 4) RT-PCR assay.^{71, 124} Total RNA samples (n=4) were obtained using the Qiagen RNeasy® Microarray Tissue Mini Kit. cDNA was generated by a BIO-RAD iScript™ cDNA Synthesis Kit. The RT-PCR reaction was carried out using the TaqMan Gene Expression Master Mix (BIO-RAD) by the 7500 Real-Time PCR System. The information of primers was shown in Table 1.

TABLE 1
Primers used for RT-PCR in the study.

| | Catalog No. |
|---|---|
| Primer | (Applied Biosystems) |
| TNF-α | Mm00443260_g1 |
| IFN-γ | Mm01168134_m1 |
| IL-4 | Mm00445259_m1 |
| IL-6 | Mm00446190_m1 |
| IL-10 | Mm01288386_m1 |
| IL-12 | Mm00434169_m1 |
| GAPDH | Mm99999915_g1 |

The depletion study of CD4⁺ and CD8⁺ T cells was performed as previously described.^{71, 124} In brief, 100 μg of either anti-CD8 (clone 53-6.72, Bioxcell), anti-CD4 (clone GK1.5, Bioxcell) or IgG (Bioxcell, polyclonal) antibodies were i.p. injected per mouse at respective schedules (FIGS. 13 and 14) before the treatment of Nano-FdUMP/Nano-Folox. Tumor growth was measured using the IVIS® Kinetics Optical System (n=4).

Combination Therapy of Nano-FdUMP and Nano-Folox with PD-L1 Blockade for CRC Liver Metastasis Mouse Model

The CRC liver metastasis mouse model was established as previously described.⁷¹ In brief, mice were anesthetized using 2.5% isoflurane, and the spleen was exteriorized, tied and sectioned. Afterwards, ˜2×10⁵ CT26-FL3 cells were injected to the distal section of the spleen. The hemi-spleen injected by CT26-FL3 cells was removed, and the other half was placed back into the cavity. Following tumor inoculation at Day 0, tumor growth was monitored using the IVIS® Kinetics Optical System. When tumor growth was reached at ˜0.5 to 1×10⁸ p/sec/cm²/sr, mice were i.v. administrated with Nano-Folox containing 1.5 mg/kg of Pt (˜4.5 mg/kg of FnA) as described in FIG. 15, which were followed by i.v. administration of Nano-FdUMP (10 mg/kg of fluorine drug) at 8 h post-injection. After this, mice were i.p. injected with or without anti-PD-L1 mAb (Bioxcell, clone 10F.9G2, 100 μg per mouse). The tumor growth was observed using the IVIS® Kinetics Optical System (n=6). Separately, one day following two injections, tumors were obtained on Day 12 for TUNEL analysis (n=4), immunofluorescence staining assay (n=4), flow cytometry (n=4) and RT-PCR experiment (n=4), as described above.

TABLE 2
Antibodies used in flow cytometry and IF microscopy experiments

| Antibody | Company | Catalog No. | Experiment | Dilution |
|---|---|---|---|---|
| Alexa Fluor ® | BD | 557959 | Flow | 1:500 |
| 700 Anti-CD8 | Bioscience | | | |
| Alexa Fluor ®647 | BioLegend | 100209 | IF/Flow | 1:500 |
| Anti-CD3 | | | | |
| PE Anti-CD3 | BioLegend | 100219 | Flow | 1:500 |
| FITC Anti-CD4 | BioLegend | 100405 | Flow | 1:500 |
| APC Anti-CD4 | BioLegend | 100411 | Flow | 1:500 |
| FITC Anti-CD44 | BioLegend | 103005 | Flow | 1:500 |
| APC Anti-CD62L | eBioscinece | 17-0621-81 | Flow | 1:500 |
| FITC Anti-CD11c | BioLegend | 117305 | Flow | 1:500 |
| Alexa Fluor ®647 | BioLegend | 107617 | Flow | 1:500 |
| Anti-MHC II | | | | |
| Alexa Fluor ®488 | BioLegend | 101217 | Flow | 1:500 |
| CD11b | | | | |
| APC Anti-Gr1 | BioLegend | 108412 | Flow | 1:500 |
| PE Anti-CD206 | BioLegend | 141705 | Flow | 1:500 |
| Alexa Fluor ®647 | BioLegend | 123121 | Flow | 1:500 |
| Anti-F4/80 | | | | |
| Alexa Fluor ®488 | BioLegend | 126406 | Flow | 1:500 |
| FoxP3 | | | | |

Data is shown in mean±standard deviation (SD). The significance between two groups was evaluated using unpaired Student's t-test (two-tailed). The significance between three or more groups was assessed using the two-way ANOVA (Bonferroni's Post-Hoc model). A log rank test was utilized for comparison in survival study. In this work, p<0.05 was considered statistically significant.

### Example 10. Preparation and Physicochemical Characterization of Nano-FdUMP

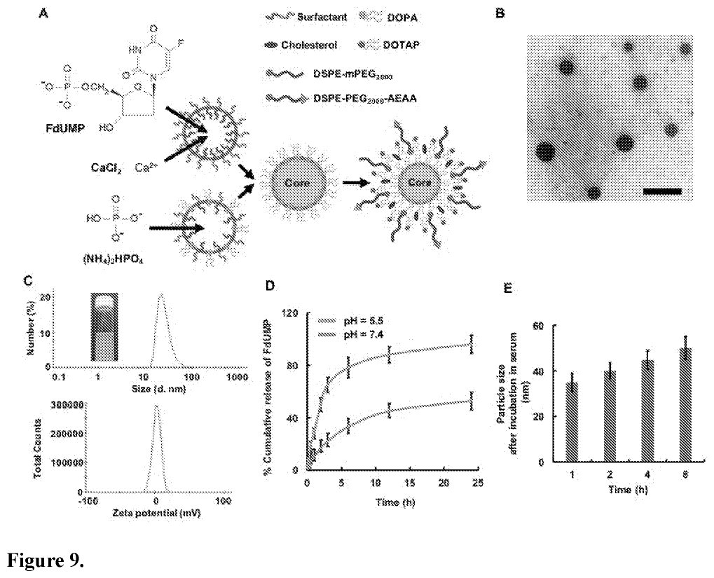

One water-in-oil microemulsion containing CaCl₂ and FdUMP was mixed with another water-in-oil microemulsion containing Na₂HPO₄, in order to generate Ca₃(PO₄)₂ amorphous precipitate in which FdUMP was entrapped (FIG. 9A). The Ca₃(PO₄)₂-FdUMP nanoprecipitate was stabilized by 1,2-dioleoyl-sn-glycero-3-phosphate (DOPA), and the nanoprecipitate was coated with 1,2-dioleoyl-3-trimethyl ammonium-propane (DOTAP), cholesterol, 1,2-distearoyl-sn-glycero-3-phosphoethanolamine-PEG₂₀₀₀ (DSPE-PEG) and DSPE-PEG-AEAA, resulting in Nano-FdUMP (FIG. 9B). Nano-FdUMP is reminiscent of other nanoformulations containing Ca₃(PO₄)₂-nucleic acid nanoprecipitate that have also been developed using nanoprecipitation process in our lab.⁸³⁻⁹¹ Nano-FdUMP illustrated nanoscale particle size (˜35 nm, polydispersity index 0.3) and neutral surface charge (˜2 mV) (FIG. 9C). The encapsulation efficiency (EE %) and loading capacity (LC %) of FdUMP in Nano-FdUMP were ˜98% and ˜38 wt %, respectively, as measured using HPLC, which were similar to EE % and LC % for FdUMP in Nano-FdUMP without AEAA.

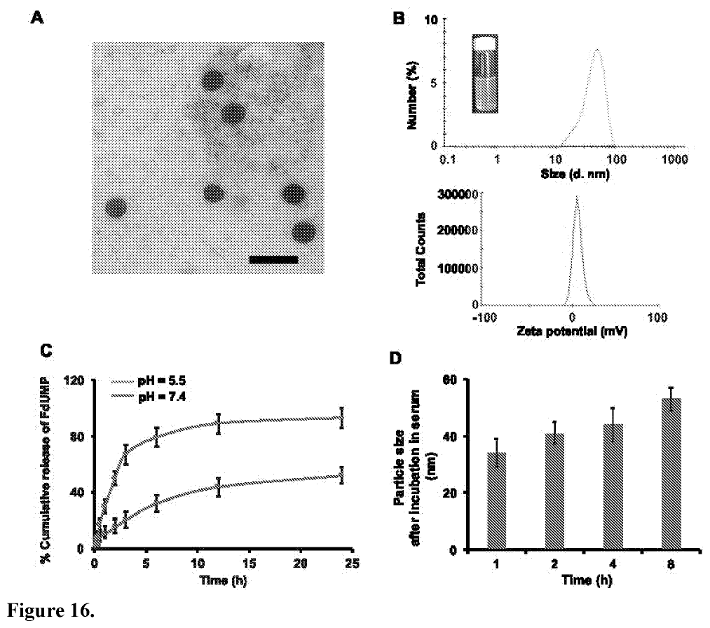

As shown in FIG. 9D, ˜50% of FdUMP were released from nanoprecipitate in Nano-FdUMP at 24 h in neutral PBS, while drug release was remarkably increased (>95%) in acidic PBS. These indicate that Nano-FdUMP showed pH-sensitive drug release, which is most likely due to the acid-sensitive feature of Ca₃(PO₄)₂.⁹² No significant aggregation (increased from ˜35 to 50 nm) was caused by nano-FdUMP in serum-containing medium up to 8 h (FIG. 9E). In addition, Nano-FdUMP without AEAA demonstrated similar morphology, particle size, surface charge, drug release and serum stability (FIG. 16) as observed for Nano-FdUMP (FIG. 9).

5-Fu can be metabolized into FdUMP within cancer cells, and FdUMP forms a complex with thymidylate synthase for inhibition of deoxythymidine monophosphate (dTMP) production.⁸² However, intracellular metabolism of 5-Fu into FdUMP is a rate-limiting process that dampens therapeutic efficacy; for example, over 80% of a single dose of 5-Fu is converted to inactive metabolites.⁹³ In addition, although 5-Fu is well tolerated, serious toxic signs are found in patients who have deficiency of dihydropyrimidine dehydrogenase, the enzyme that is responsible for metabolism of 5-Fu. This toxicity is due to 5-Fu but not metabolites.⁹³ In order to bypass these resistances, FdUMP, instead of 5-Fu, was formulated using our AEAA-targeted PEGylated NP (Nano-FdUMP) (FIG. 9A). Free FdUMP, being a nucleoside phosphate, is impermeable into cells,⁹⁴ while Nano-FdUMP can efficiently carry the impermeable FdUMP into cancer cells (see below results). Of note, a variety of nanoformulations have been recently developed for delivery of 5-Fu in tumor-bearing mouse models.⁹⁵⁻⁹⁷ For example, Li et al. produced a polymeric NP of poly(γ-benzyl-L-glutamate) (PBLG) and PEG for delivery of 5-Fu in subcutaneous CRC mouse model, however, EE % and LC % were only ˜61% and ˜27%, respectively.⁹⁵ Safwat and colleagues also developed a gold NP-based system for delivery of 5-Fu in skin cancer mouse model, but EE % was less than 70%.⁹⁶ In addition, Kazi and coworkers designed a poly(lactic-co-glycolic acid) (PLGA)-based NP for delivery of 5-Fu in melanoma mouse model, however, EE % and LC % were only ˜56% and ˜2%, respectively.⁹⁷ Here, Nano-FdUMP achieved significantly higher EE % (˜98%) and LC % (˜38%) of 5-Fu metabolite than these studies. Taken together, our results indicated that Nano-FdUMP provides great advantages over these previously reported 5-Fu nanoformulations, from mechanism of action, drug encapsulation efficiency and loading capacity points of view.

### Example 11. In Vitro Anticancer Effects of Nano-FdUMP

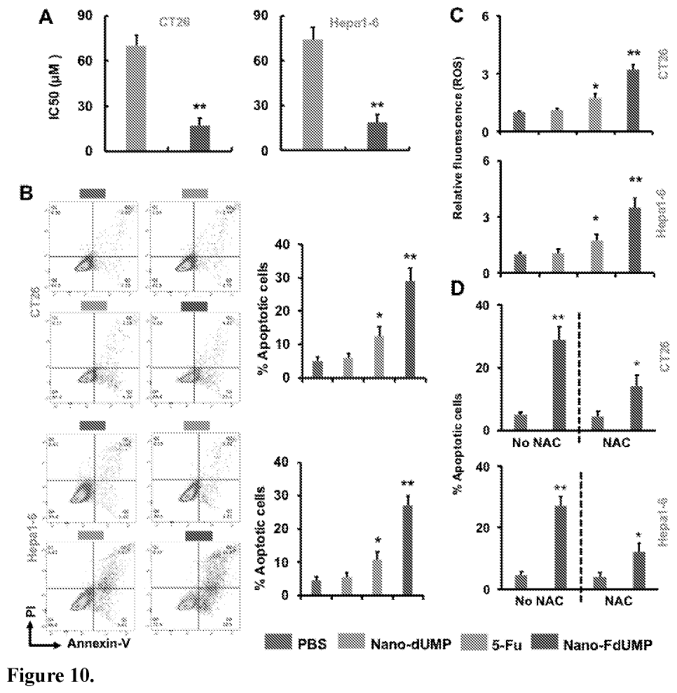

Nano-FdUMP caused significantly higher cytotoxicity (IC₅₀≤20 μM, 24 h incubation; p<0.01) in mouse CRC (CT26) and HCC (Hepa1-6) cell lines relative to 5-Fu (IC₅₀≈70 μM, 24 h incubation) (FIG. 10A). Nano-dUMP, in which FdUMP was replaced by 2′-deoxyuridine 5′-monophosphate (dUMP), was chosen as negative control. Of note, IC₅₀ of Nano-dUMP could not be determined under the conditions tested, demonstrating that neither dUMP nor AEAA-targeted formulation was cytotoxic. In addition, no significant difference in apoptosis of CT26 and Hepa1-6 cells was observed between Nano-dUMP and PBS (FIG. 10B), while Nano-FdUMP induced significantly higher level of apoptosis (p<0.01, 24 h incubation) as compared to Nano-dUMP and 5-Fu (FIG. 10B). These indicate that cytotoxic and apoptotic effects of Nano-FdUMP were mainly due to delivery of fluorine drug using AEAA-targeted nanoformulation.

The capacity of Nano-FdUMP to induce ROS was subsequently assessed in CT26 and Hepa1-6 cells (FIG. 10C). Results showed that no significant difference in ROS formation was found in cancer cells between Nano-dUMP and PBS, while Nano-FdUMP caused significantly higher level of ROS (p<0.01, 24 h) than Nano-dUMP and 5-Fu (FIG. 10C). Glutathione (GSH) is known as the primary endogenous antioxidant, and plays a key role in neutralization of intracellular ROS by direct and indirect scavenging.⁹⁸ As the synthesis of GSH is mainly relied on L-cysteine,⁹⁹ and N-acetyl-L-cysteine (NAC) is the acetylated variant (a precursor) of L-cysteine,¹⁰⁰ NAC can be used to provide L-cysteine for GSH production. Here, NAC was used to investigate the role of ROS achieved by Nano-FdUMP in the induction of apoptosis (FIG. 10D). The apoptotic efficacy of Nano-FdUMP was significantly reduced (p<0.01, 24 h) from ˜30% to ˜15% when cancer cells were pretreated with NAC (FIG. 10D). Results in FIGS. 10C and 10D show that the apoptosis of CRC and HCC cells is, at least, in part due to ROS formation achieved by Nano-FdUMP.

### Example 12. Synergistic ICD Effects of Nano-FdUMP and Nano-Folox

ICD-associated immunogenicity can be evoked by ROS,⁷⁸ and the efficacy of ICD may be improved by ROS-inducing strategies.⁷⁹⁻⁸¹ Nano-Folox results in OxP-mediated ICD for anticancer immune response.⁷¹ Here, synergistic ICD effects of Nano-FdUMP and Nano-Folox were assessed using CT26 and Hepa1-6 cells in terms of ICD hallmarks, namely exposure of calreticulin (CRT), secretion of adenosine triphosphate (ATP), and release of high mobility group protein B1 (HMGB1).⁷⁸

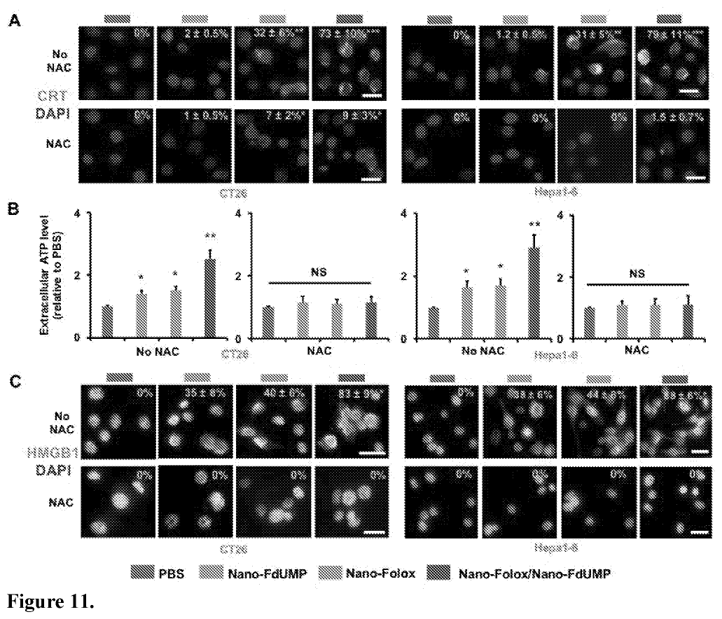

Results in FIG. 11A show that no significant difference in exposure of CRT was observed between Nano-FdUMP and PBS, most likely due to the inefficiency of 5-Fu or metabolites in facilitating the translocation of CRT.¹⁰¹ In contrast, Nano-Folox was able to mediate significantly efficient CRT exposure (p<0.01, ˜31 to 32%) onto the cell membrane (FIG. 11A). Notably, combination of Nano-FdUMP and Nano-Folox further improved translocation of CRT (p<0.001, ˜73 to 79%) (FIG. 11A). Although 5-Fu or metabolites cannot effectively induce CRT exposure, they may facilitate release of ATP and secretion of HMGB1.¹⁰¹ Indeed, as compared to PBS, Nano-FdUMP significantly activated secretion of ATP into extracellular milieu (p<0.05), which was similar to results obtained by Nano-Folox (FIG. 11B). Of note, combination of two nanoformulations further enhanced secretion of ATP (p<0.01) (FIG. 11B). Moreover, Nano-FdUMP significantly enhanced release of HMGB1 from the nucleus into the cytoplasm as compared to PBS, which was similar to results found in Nano-Folox (FIG. 11C). Notably, combination of two nanoformulations further promoted release of HMGB1 (p<0.05) (FIG. 11C). These results demonstrated that Nano-FdUMP could synergize with Nano-Folox for improved ICD effects.

It is worth noting that when cancer cells were pretreated with NAC, the activity of ICD hallmarks was significantly suppressed in either Nano-FdUMP, Nano-Folox, or combination (FIG. 11), indicating that 1) the production of ROS is critical for Nano-Folox-mediated ICD induction, most likely due to the fact that OxP induces ICD via both endoplasmic reticulum (ER) stress and ROS generation; 2) the critical role of ROS achieved by Nano-FdUMP in promoting ICD effects of Nano-Folox.

### Example 13: Pharmacokinetics and Biodistribution of Nano-FdUMP

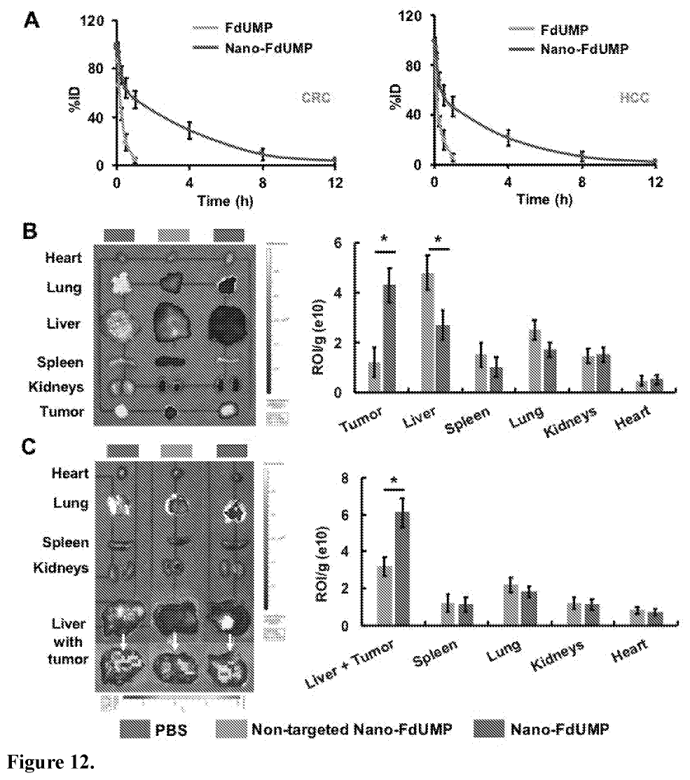

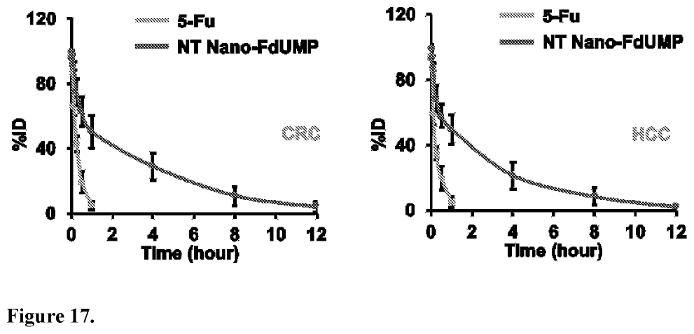

Generally, short blood circulation and quick renal elimination are caused by i.v. administration of 5-Fu.¹⁰² PEGylated nanoformulation may significantly increase half-life of chemotherapeutics in the bloodstream.¹⁰³ Here, the half-life of Nano-FdUMP was determined using orthotopic CT26-FL3 derived CRC and Hepa1-6-Luc derived HCC mouse models, respectively (FIG. 12A). Results showed that the concentration of fluorine drug in the plasma decreased rapidly, and a minor level was detected at 1 h post injection (t_{1/2}≈6 min and 5 min for FdUMP in CRC and HCC models, respectively; FIG. 12A). In contrast, fluorine drug in Nano-FdUMP was more slowly eliminated from the plasma (t_{1/2}≈1.6 h and 1.4 h for FdUMP in CRC and HCC models, respectively; FIG. 12A). In addition, Nano-FdUMP without AEAA demonstrated similar half-lives (FIG. 17) as recorded by Nano-FdUMP with AEAA (FIG. 12A). These results confirmed that half-life of fluorine drug was significantly improved by Nano-FdUMP, which is most likely due to the PEG modification.

The tissue distribution of Nano-FdUMP was also investigated using orthotopic CRC and HCC mouse models, respectively. Following i.v. injection of DiD-labeled nanoformulations, tumors and major tissues were ex vivo imaged using the IVIS® Kinetics Optical System (FIGS. 12B and 12C). In CRC model, AEAA-targeted Nano-FdUMP achieved significantly higher retention in tumors (˜2.5 fold; p<0.05) but significantly less accumulation in the liver (˜2 fold; p<0.05) than non-targeted nanoformulation (FIG. 12B). In HCC model, AEAA-targeted nanoformulation was specifically accumulated inside liver tumor, which was confirmed by colocalization of NPs (fluorescence imaging from DiD dye) and tumor tissue (bioluminescence imaging from visible light produced by luciferase in tumor cells) (FIG. 12C). However, non-targeted nanoformulation was mainly found in healthy liver rather than the tumor (FIG. 12C). These confirmed that AEAA-targeted nanoformulation significantly improved tumor accumulation and alleviated non-specific tissue distribution.

Cancer patients suffer from time-consuming schedule of FOLFOX, and serious side effects are caused by such excessive treatment.^{68,69} Nano-Folox can prolongs blood circulation and enhance tumor accumulation of platinum drug and FnA.⁷¹ As shown in FIG. 12, Nano-FdUMP significantly increased half-life and tumor accumulation of fluorine drug. Therefore, it suggests that combination of Nano-FdUMP and Nano-Folox provide a strategy with reduced treatment cycle and lower dose, which sufficiently achieve therapeutic outcomes as compared to conventional FOLFOX.

### Example 14: Combination of Nano-FdUMP and Nano-Folox for Synergistic Chemo-Immunotherapy in Orthotopic CRC and HCC Mouse Models

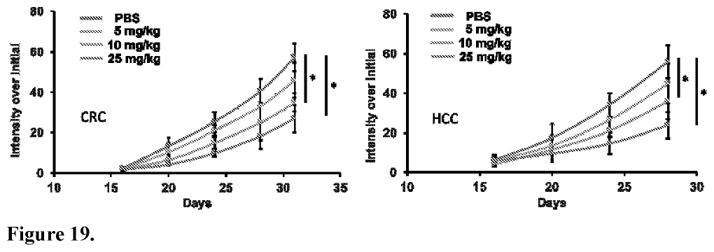

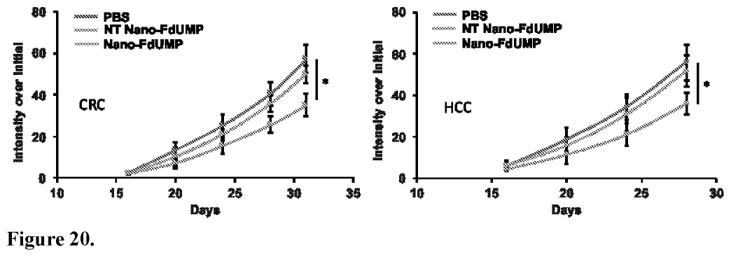

The in vivo toxicity of Nano-FdUMP was first assessed in healthy mice (FIG. 18). No significant body weight loss was found in Nano-FdUMP at 5, 10 and 25 mg/kg FdUMP; however, Nano-FdUMP at 50 mg/kg of FdUMP caused slight body weight loss (FIG. 18). In addition, toxic signs (e.g. hunched posture, ruffled hair coat, and reluctance to move) were observed in mice treated with Nano-FdUMP at higher dose (50 mg/kg) but not at lower doses (5, 10 and 25 mg/kg) (FIG. 18). Furthermore, the antitumor efficacy of Nano-FdUMP at different doses was assessed in orthotopic CT26-FL3 derived CRC and Hepa1-6-Luc derived HCC mouse models, respectively (FIG. 19). The antitumor efficacy of Nano-FdUMP was dose-dependent, and the growth of CRC and HCC was significantly slowed down by Nano-FdUMP containing 10 and 25 mg/kg of FdUMP (FIG. 19). In addition, no antitumor efficacy was achieved by non-targeted Nano-FdUMP as compared to PBS, but AEAA-targeted Nano-FdUMP significantly slowed down tumor growth (p<0.05) than non-targeted nanoformulation (FIG. 20), confirming AEAA-mediated antitumor effect. Based on these results, Nano-FdUMP containing 10 mg/kg of FdUMP was chosen for following studies of combination therapy (FIGS. 13 and 14).

Previously, “Nano-Folox and free 5-Fu” demonstrated significantly improved therapeutic outcome than FOLFOX (free drugs, used as positive control).⁷¹ Thus, “Nano-Folox and free 5-Fu” was chosen as positive control here. As shown in FIGS. 13A and 13B, combination of Nano-FdUMP (10 mg/kg of FdUMP) and Nano-Folox (1.5 mg/kg of platinum drug and 4.5 mg/kg of FnA) demonstrated significantly improved antitumor efficacy (p<0.01) than Nano-FdUMP alone, Nano-FdUMP with OxP and FnA, and Nano-Folox with 5-Fu (10 mg/kg). It is worth noting that combination of Nano-FdUMP and Nano-Folox provided long-term survival in 5 out of 6 mice, which was significantly improved (p<0.001) than PBS [median survival (MS)=40 days)], Nano-FdUMP (MS=45 days), Nano-FdUMP with OxP and FnA (MS=49 days), and Nano-Folox with 5-Fu (MS=56 days) (FIG. 13C).

Nano-Folox causes platinum-DNA-adducts for apoptosis, and the apoptotic efficacy was further enhanced when combined with 5-Fu. Here, immunofluorescence results showed that combination of Nano-FdUMP and Nano-Folox significantly (p<0.05) induced apoptosis in tumors (˜32%) relative to PBS (˜0.3%), Nano-FdUMP alone (˜2%), Nano-FdUMP with OxP and 5-Fu (˜4%), and Nano-Folox with 5-Fu (˜10%) (FIG. 13D). The enhanced apoptotic efficacy is most likely due to the fact that 1) targeted delivery of 5-Fu metabolite was achieved using AEAA-targeted nanoformulation; 2) the efficacy of 5-Fu metabolite was promoted by FnA released from Nano-Folox; 3) 5-Fu metabolite/FnA further enhanced apoptotic effect with OxP derivative released from Nano-Folox.⁷¹ Moreover, combination of two nanoformulations induced ICD for a shift from a “cold” tumor microenvironment (TME) into a “hot” T cell-inflamed one (˜28% T cell infiltration; p<0.01) as compared to the other controls (FIG. 13E). The TME remodeling achieved by the combination strategy was further supported by increment of immunostimulatory factors and reduction of immunosuppressive factors (FIGS. 13F and 13G). For example, CD8⁺ T cells, CD4⁺ T cells and dendritic cells (DCs) were significantly activated in tumors by the combination strategy (FIG. 13F), which were accompanied with upregulation of IFN-γ, TNF-α and IL-12, three cytokines for activation of antitumor immunity (FIG. 13G).¹⁰⁴ On the contrary, myeloid derived suppressor cells (MDSCs), regulatory T cells (Tregs) and tumor-associated macrophages (M2) were significantly decreased in tumors by the combination strategy (FIG. 13F), which were accompanied with downregulation of immunosuppressive cytokines such as IL-4, IL-6 and IL-10 (FIG. 13G).¹⁰⁵ It is known that ICD-associated antitumor immunity is essentially relied on the activation of effector T cells for killing tumor cells.⁸⁰ In order to confirm the immunotherapeutic mechanism, orthotopic CRC animals were administrated with Nano-FdUMP/Nano-Folox following the depletion of either CD8⁺ or CD4⁺ T cells with corresponding monoclonal anti-CD8 or -CD4 antibody, respectively (FIG. 13H). Consequently, the antitumor efficacy of Nano-FdUMP/Nano-Folox was significantly suppressed (p<0.01) following the injection of these antibodies, but not the isotype IgG (FIG. 13H), confirming the critical role of effector T cells for antitumor immunity mediated by the combination strategy. Therefore, synergistic immunologic effects in FIG. 13 are most likely due to the fact that Nano-FdUMP significantly promoted Nano-Folox-mediated ICD efficacy.

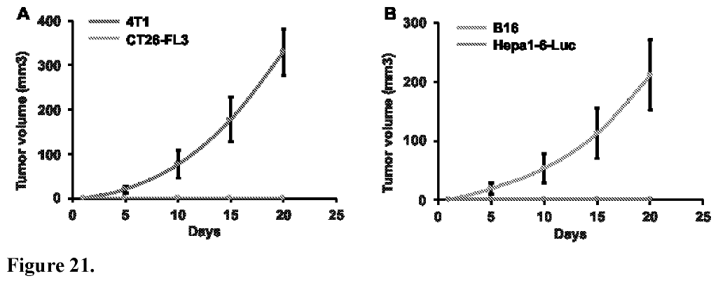

FOLFOX demonstrates great potential for the generation of memory T cells,¹⁰⁶ and IL-12 plays key role in activation and proliferation of antigen-specific memory T cells.^{107, 108} Indeed, memory CD8⁺ and CD4⁺ T cells were successfully activated in tumors following treatment of Nano-FdUMP/Nano-Folox (FIG. 13F). In order to confirm tumor-specific memory response, tumor-free mice “cured” by the treatment of Nano-FdUMP/Nano-Folox were rechallenged with 4T1 and CT26-FL3 cells (FIG. 21). Results showed that 4T1 breast tumor growth was not affected, while CT26-FL3 tumor growth was significantly inhibited in same animals (FIG. 21). These further confirmed that the combination approach has potential for induction of tumor-specific memory response against CRC, facilitating long-term survival in mice (FIG. 13C).

In addition, significantly improved antitumor efficacy (p<0.01) was also achieved by the combination strategy in orthotopic HCC mice than the other controls (FIGS. 14A and 14B), which facilitated long-term survival in 4 out of 6 mice (FIG. 14C). The antitumor outcome mainly resulted from chemo-immunotherapeutic effects including apoptosis (FIG. 14D) and TME remodeling (FIG. 14E) achieved by the combination strategy. The TME remodeling was supported by increment of immunostimulatory factors and reduction of immunosuppressive factors (FIGS. 14F and 14G). Following treatment of Nano-FdUMP/Nano-Folox, CD8⁺ T cells, CD4⁺ T cells and DCs were significantly activated in tumors (FIG. 14F), which were accompanied with increase of IFN-γ, TNF-α and IL-12 (FIG. 14G). In contrast, MDSCs, Tregs and M2 cells were significantly decreased in tumors (FIG. 13F), which were accompanied with alleviation of IL-4, IL-6 and IL-10 (FIG. 14G). In addition, the antitumor efficacy of Nano-FdUMP/Nano-Folox was also significantly suppressed (p<0.01) in HCC mouse model following the pretreatment of anti-CD8 or anti-CD4 antibodies (FIG. 14H), confirming the critical roles of effector T cells for antitumor immunity mediated by the combination strategy. Furthermore, tumor-free mice “cured” by combined approach were rechallenged with B16 melanoma and Hepa1-6-Luc cells (FIG. 21). Results showed that B16 tumor growth was not affected in cured mice, while Hepa1-6-Luc tumor growth was significantly suppressed in same animals (FIG. 21). These results showed that the combination approach also has potential for induction of tumor-specific memory response against HCC, facilitating long-term survival in mice (FIG. 14C).

In addition, no toxic signs were caused by the combination strategy as compared to PBS, which was confirmed by analysis of body weight, hematological toxicity, and liver/kidney damage in healthy mice (FIG. 22). Taken together, the “Nano-FdUMP+Nano-Folox” strategy could achieve synergistic chemo-immunotherapeutic efficacy against CRC and HCC for long-term survival in mice, without causing significant side effects.

### Example 15: Blockade of PD-L1 Enhanced Combination of Nano-FdUMP and Nano-Folox for Inhibition of Liver Metastasis

FOLFOX has been used for patients with unresectable CRC liver metastases;⁶⁷ however, therapeutic outcome is still poor due to fast tumor progression and high relapse rate. Here, the “Nano-FdUMP+Nano-Folox” strategy was further applied to treat mice with experimental liver metastasis (FIG. 15). This tumor-bearing model closely reproduces the aggressive pattern of CRC at metastatic stage.¹⁰⁹ As shown in FIGS. 15A and 15B, the combined approach was able to significantly (p<0.01) slow down tumor growth in mice as compared to PBS, which was accompanied by apoptosis (˜11%) (FIG. 15D) and T cell infiltration (˜12%) (FIG. 15E). However, no long-term survival (MS=48 days) was achieved by the combination strategy after dosing (FIG. 15C). Blockade of PD-L1 significantly improves overall survival of animals grafted by CRC liver metastasis in combination with “Nano-Folox+5-Fu”.⁷¹ Therefore, it was hypothesized that anti-PD-L1 mAb may further advance the “Nano-FdUMP+Nano-Folox” strategy. Indeed, the combination of Nano-FdUMP/Nano-Folox and anti-PD-L1 mAb significantly inhibited liver metastases (p<0.01) as compared to either Nano-FdUMP/Nano-Folox or anti-PD-L1 mAb (FIGS. 15A and 15B), which was accompanied with improved apoptosis (˜40%) (FIG. 15D) and T cell infiltration (˜40%) (FIG. 15E). Of note, combination of Nano-FdUMP/Nano-Folox and anti-PD-L1 mAb was able to provide long-term survival in 5 out of 6 mice (FIG. 15C). It is most likely due to the fact that combination of Nano-FdUMP/Nano-Folox and anti-PD-L1 mAb significantly (p<0.05 and p<0.01) increased the amount of effector/memory T cells and DCs (FIG. 15F), upregulated the expression of IFN-γ and IL-12 (FIG. 15G) and reduced the level of IL-4, IL-6, and IL-10 (FIG. 15G), as compared to either FdUMP/Nano-Folox or anti-PD-L1 mAb. These indicated that FdUMP/Nano-Folox may significantly remodel the immunosuppressive TME for enhanced antitumor outcome in combination with immune checkpoint blockade, potentially providing a chemo-immunotherapeutic strategy for metastatic CRC.

### Example 16

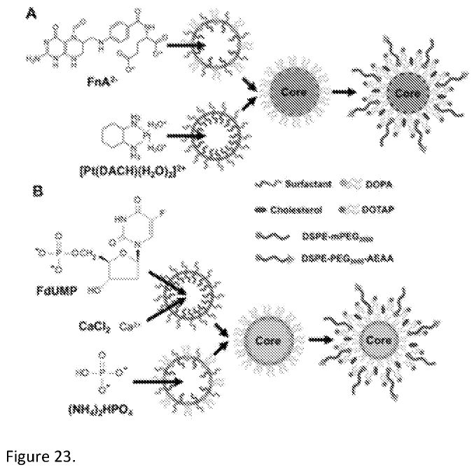

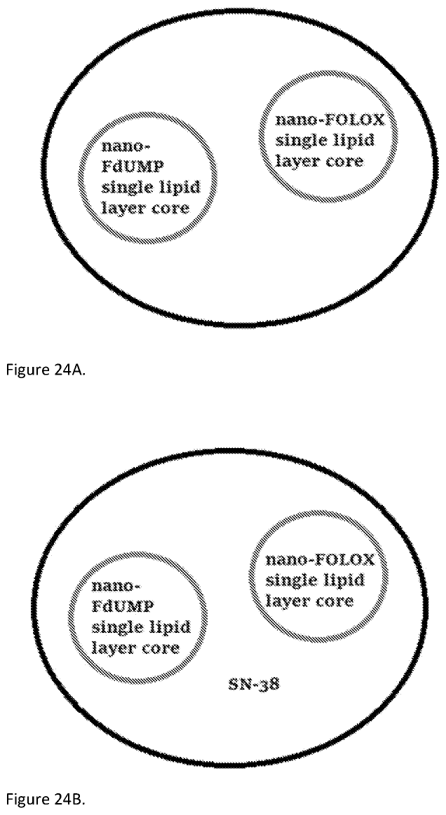

FOLFOX is the combination therapy using three drugs together: Folinic acid, 5-FU and Oxaliplatin. Previous disclosure described nano-FOLOX and nano-FdUMP, and their use in combination to treat colorectal and liver cancers. An important intermediate of both nano-formulations is the “Core” structure described in FIGS. 23A and B. These cores are stabilized by using a phospholipid, i.e. dioleoyl phosphatidic acid (DOPA). Hence, the cores are hydrophobic in both cases. These purified cores are hydrophobic and can be dissolved and stored in CHCl₃ for at least a year. We will encapsulate these cores in a polymer emulsion containing PLGA, PLGA-PEG and PLGA-PEG-AEAA (4:4:2 molar ratio). Cores, at different ratios, and polymers will be dissolved in tetrahydrofuran (THF) and added dropwise into 2 mL of water under constant stirring at room temperature. The resulting NP suspension will be stirred uncovered for 6 h at room temperature to remove THF. The NPs will be further purified by ultrafiltration. The PLGA NPs will then be re-suspended, washed with water, and centrifuged at 14,000 rpm for 20 min to remove free lipids and micelles, re-suspended and centrifuged again at 800 rpm to remove any nanocore aggregates. Drug loading and encapsulation efficiency of FOLOX will be measured using Inductively Coupled Plasma-Mass Spectroscopy (ICP-MS). Loading and encapsulation of FdUMP will be measured by Ultraviolet-Visible Spectrometry. Formulations with different ratios of the two cores will be manufactured. Since these PLGA nano-emulsions contain all three drugs. This nano-formulation is referred to herein as “nano-FOLFOX and is depicted in FIG. 24a”

### Example 17: Combination of Nano-FdUMP and Nano-Folox and Irinotecan

For certain cancers, such as the pancreatic ductal adenocarcinoma, one additional drug, i.e. irinotecan, is often added to the combination therapy regimen. The combination therapy is called FOLFIRINOX. The formulation is depicted in FIG. 24b. Leveraging the chemistry described herein can result in the preparation of a combination nanoparticle complex. A polymer exterior, such as PLGA or PLGA-PEG-AEAA The 4 drugs are Folinic acid, 5-FU, Irinotecan and Oxaliplatin. An active metabolite of irinotecan, i.e. SN-38, can be added to the THF solution containing both cores described above. SN-38 is hydrophobic and is soluble in THF. The resulting nanoparticles contain 4 drugs, i.e. Folinic acid, FdUMP (an active metabolite of 5-FU), oxaliplatin and SN-38 (an active metabolite of irinotecan). This nano-formulation is referred to herein as “nano-FOLFIRINOX”.

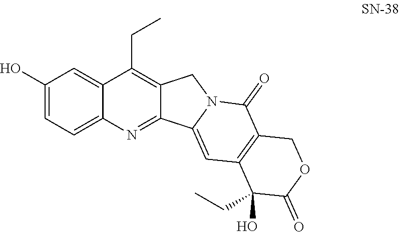 [SMILES: CCc1c2c(nc3ccc(O)cc13)-c1cc3c(c(=O)n1C2)COC(=O)[C@]3(O)CC]

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the foregoing list of embodiments and appended claims. Although specific terms are employed herein, they are used in a generic and descriptive sense only and not for purposes of limitation.

### BIBLIOGRAPHY

1. Arnold M, Sierra M S, Laversanne M, Soerjomataram I, Jemal A, Bray F. Global patterns and trends in colorectal cancer incidence and mortality. Gut 2017, 66(4): 683-691.
2. Ciombor K K, Wu C, Goldberg R M. Recent therapeutic advances in the treatment of colorectal cancer. Annu Rev Med 2015, 66: 83-95.
3. Tournigand C, Cervantes A, Figer A, Lledo G, Flesch M, Buyse M, et al. OPTIMOX1: a randomized study of FOLFOX4 or FOLFOX7 with oxaliplatin in a stop-and-Go fashion in advanced colorectal cancer—a GERCOR study. J Clin Oncol 2006, 24(3): 394-400.
4. Allegra C J, Yothers G, O'Connell M J, Sharif S, Petrelli N J, Lopa S H, et al. Bevacizumab in stage II-III colon cancer: 5-year update of the National Surgical Adjuvant Breast and Bowel Project C-08 trial. J Clin Oncol 2013, 31(3): 359-364.
5. Nordlinger B, Sorbye H, Glimelius B, Poston G J, Schlag P M, Rougier P, et al. Perioperative FOLFOX4 chemotherapy and surgery versus surgery alone for resectable liver metastases from colorectal cancer (EORTC 40983): long-term results of a randomised, controlled, phase 3 trial. Lancet Oncol 2013, 14(12): 1208-1215.
6. Guo S, Wang Y, Miao L, Xu Z, Lin C M, Zhang Y, et al. Lipid-coated Cisplatin nanoparticles induce neighboring effect and exhibit enhanced anticancer efficacy. ACS Nano 2013, 7(11): 9896-9904.
7. Guo S, Miao L, Wang Y, Huang L. Unmodified drug used as a material to construct nanoparticles: delivery of cisplatin for enhanced anti-cancer therapy. J Control Release 2014, 174: 137-142.
8. Guo S, Wang Y, Miao L, Xu Z, Lin C H, Huang L. Turning a water and oil insoluble cisplatin derivative into a nanoparticle formulation for cancer therapy. Biomaterials 2014, 35(26): 7647-7653.
9. Kim G P, Erlichman C. Oxaliplatin in the treatment of colorectal cancer. Expert Opin Drug Metab Toxicol 2007, 3(2): 281-294.
10. Jerremalm E, Wallin I, Ehrsson H. New insights into the biotransformation and pharmacokinetics of oxaliplatin. J Pharm Sci 2009, 98(11): 3879-3885.
11. Graham M A, Lockwood G F, Greenslade D, Brienza S, Bayssas M, Gamelin E. Clinical pharmacokinetics of oxaliplatin: a critical review. Clin Cancer Res 2000, 6(4): 1205-1218.
12. Jerremalm E, Hedeland M, Wallin I, Bondesson U, Ehrsson H. Oxaliplatin degradation in the presence of chloride: identification and cytotoxicity of the monochloro monooxalato complex. Pharm Res 2004, 21(5): 891-894.
13. Di Francesco A M, Ruggiero A, Riccardi R. Cellular and molecular aspects of drugs of the future: oxaliplatin. Cell Mol Life Sci 2002, 59(11): 1914-1927.
14. Levina A, Mitra A, Lay P A. Recent developments in ruthenium anticancer drugs. Metallomics 2009, 1(6): 458-470.
15. Martinez-Balibrea E, Martinez-Cardus A, Gines A, Ruiz de Porras V, Moutinho C, Layos L, et al. Tumor-Related Molecular Mechanisms of Oxaliplatin Resistance. Mol Cancer Ther 2015, 14(8): 1767-1776.
16. Longley D B, Harkin D P, Johnston P G. 5-Fluorouracil: Mechanisms of action and clinical strategies. Nature Reviews Cancer 2003, 3(5): 330-338.
17. Park J G, Collins J M, Gazdar A F, Allegra C J, Steinberg S M, Greene R F, et al.

Enhancement of Fluorinated Pyrimidine-Induced Cyto-Toxicity by Leucovorin in Human Colorectal-Carcinoma Cell-Lines. J Natl Cancer I 1988, 80(19): 1560-1564.
18. Nadal J C, Van Groeningen C J, Pinedo H M, Peters G J. In vivo potentiation of 5-fluorouracil by leucovorin in murine colon carcinoma. Biomed Pharmacother 1988, 42(6): 387-393.
19. de Gramont A, Figer A, Seymour M, Homerin M, Hmissi A, Cassidy J, et al. Leucovorin and fluorouracil with or without oxaliplatin as first-line treatment in advanced colorectal cancer. Journal of Clinical Oncology 2000, 18(16): 2938-2947.
20. Khiati S, Luvino D, Oumzil K, Chauffert B, Camplo M, Barthelemy P. Nucleoside-lipid-based nanoparticles for cisplatin delivery. ACS Nano 2011, 5(11): 8649-8655.
21. Zhao Y, Alakhova D Y, Kim J O, Bronich T K, Kabanov A V. A simple way to enhance Doxil (R) therapy: Drug release from liposomes at the tumor site by amphiphilic block copolymer. Journal of Controlled Release 2013, 168(1): 61-69.
22. Zhang Y, Davis C, Ryan J, Janney C, Pena M M O. Development and characterization of a reliable mouse model of colorectal cancer metastasis to the liver. Clin Exp Metastas 2013, 30(7): 903-918.
23. Liu Q, Chen F Q, Hou L, Shen L M, Zhang X Q, Wang D G, et al. Nanocarrier-Mediated Chemo-Immunotherapy Arrested Cancer Progression and Induced Tumor Dormancy in Desmoplastic Melanoma. Acs Nano 2018, 12(8): 7812-7825.
24. Liu Q, Zhu H, Tiruthani K, Shen L, Chen F, Gao K, et al. Nanoparticle-Mediated Trapping of Wnt Family Member 5A in Tumor Microenvironments Enhances Immunotherapy for B-Raf Proto-Oncogene Mutant Melanoma. ACS Nano 2018, 12(2): 1250-1261.
25. Shen L M, Li J J, Liu Q, Song W T, Zhang X Q, Tiruthani K, et al. Local Blockade of Interleukin 10 and C-X-C Motif Chemokine Ligand 12 with Nano-Delivery Promotes Antitumor Response in Murine Cancers. Acs Nano 2018, 12(10): 9830-9841.
26. An S, Tiruthani K, Wang Y, Xu L G, Hu M Y, Li J J, et al. Locally Trapping the C-C Chemokine Receptor Type 7 by Gene Delivery Nanoparticle Inhibits Lymphatic Metastasis Prior to Tumor Resection. Small 2019, 15(9).
27. Miao L, Li J J, Liu Q, Feng R, Das M, Lin C M, et al. Transient and Local Expression of Chemokine and Immune Checkpoint Traps To Treat Pancreatic Cancer. Acs Nano 2017, 11(9): 8690-8706.
28. Das M, Shen L M, Liu Q, Goodwin T J, Huang L. Nanoparticle Delivery of RIG-I Agonist Enables Effective and Safe Adjuvant Therapy in Pancreatic Cancer. Mol Ther 2019, 27(3): 507-517.
29. Hu K L, Miao L, Goodwin T J, Li J, Liu Q, Huang L. Quercetin Remodels the Tumor Microenvironment To Improve the Permeation, Retention, and Antitumor Effects of Nanoparticles. Acs Nano 2017, 11(5): 4916-4925.
30. Song W, Shen L, Wang Y, Liu Q, Goodwin T J, Li J, et al. Synergistic and low adverse effect cancer immunotherapy by immunogenic chemotherapy and locally expressed P D-L1 trap. Nat Commun 2018, 9(1): 2237.
31. Song W, Tiruthani K, Wang Y, Shen L, Hu M, Dorosheva 0, et al. Trapping of Lipopolysaccharide to Promote Immunotherapy against Colorectal Cancer and Attenuate Liver Metastasis. Adv Mater 2018, 30(52): e1805007.
32. Johnstone T C, Suntharalingam K, Lippard S J. The Next Generation of Platinum Drugs: Targeted Pt(I I) Agents, Nanoparticle Delivery, and Pt(I V) Prodrugs. Chem Rev 2016, 116(5): 3436-3486.
33. Jung Y, Lippard S J. Direct cellular responses to platinum-induced DNA damage. Chem Rev 2007, 107(5): 1387-1407.
34. Galluzzi L, Buque A, Kepp O, Zitvogel L, Kroemer G. Immunogenic cell death in cancer and infectious disease. Nat Rev Immunol 2017, 17(2): 97-111.
35. Fucikova J, Kralikova P, Fialova A, Brtnicky T, Rob L, Bartunkova J, et al. Human Tumor Cells Killed by Anthracyclines Induce a Tumor-Specific Immune Response. Cancer Res 2011, 71(14): 4821-4833.
36. Menger L, Vacchelli E, Adjemian S, Martins I, Ma Y T, Shen S S, et al. Cardiac Glycosides Exert Anticancer Effects by Inducing Immunogenic Cell Death. Sci Transl Med 2012, 4(143).
37. Krysko D V, Garg A D, Kaczmarek A, Krysko O, Agostinis P, Vandenabeele P. Immunogenic cell death and DAMPs in cancer therapy. Nature Reviews Cancer 2012, 12(12): 860-875.
38. Kroemer G, Galluzzi L, Kepp O, Zitvogel L. Immunogenic cell death in cancer therapy. Annu Rev Immunol 2013, 31: 51-72.
39. Massari C, Brienza S, Rotarski M, Gastiaburu J, Misset J L, Cupissol D, et al. Pharmacokinetics of oxaliplatin in patients with normal versus impaired renal function. Cancer Chemother Pharmacol 2000, 45(2): 157-164.
40. Andre T, Boni C, Navarro M, Tabernero J, Hickish T, Topham C, et al. Improved overall survival with oxaliplatin, fluorouracil, and leucovorin as adjuvant treatment in stage I I or III colon cancer in the MOSAIC trial. J Clin Oncol 2009, 27(19): 3109-3116.
41. Limani P, Linecker M, Kachaylo E, Tschuor C, Kron P, Schlegel A, et al. Antihypoxic Potentiation of Standard Therapy for Experimental Colorectal Liver Metastasis through Myo-Inositol Trispyrophosphate. Clin Cancer Res 2016, 22(23): 5887-5897.
42. Robinson S M, Mann D A, Manas D M, Oakley F, Mann J, White S A. The potential contribution of tumour-related factors to the development of FOLFOX-induced sinusoidal obstruction syndrome. Br J Cancer 2013, 109(9): 2396-2403.
43. Musetti S, Huang L. Nanoparticle-Mediated Remodeling of the Tumor Microenvironment to Enhance Immunotherapy. ACS Nano 2018, 12(12): 11740-11755.
44. Dong H, Strome S E, Salomao D R, Tamura H, Hirano F, Flies D B, et al. Tumor-associated B7-H₁ promotes T-cell apoptosis: a potential mechanism of immune evasion. Nat Med 2002, 8(8): 793-800.
45. Le D T, Durham J N, Smith K N, Wang H, Bartlett B R, Aulakh L K, et al. Mismatch repair deficiency predicts response of solid tumors to PD-1 blockade. Science 2017, 357(6349): 409-413.
46. Overman M J, McDermott R, Leach J L, Lonardi S, Lenz H J, Morse M A, et al. Nivolumab in patients with metastatic DNA mismatch repair-deficient or microsatellite instability-high colorectal cancer (CheckMate 142): an open-label, multicentre, phase 2 study. Lancet Oncology 2017, 18(9): 1182-1191.
47. Kasuya H, Kuruppu D K, Donahue J M, Choi E W, Kawasaki H, Tanabe K K. Mouse models of subcutaneous spleen reservoir for multiple portal venous injections to treat liver malignancies. Cancer Res 2005, 65(9): 3823-3827.
48. Castle J C, Loewer M, Boegel S, de Graaf J, Bender C, Tadmor A D, et al. Immunomic, genomic and transcriptomic characterization of CT26 colorectal carcinoma. Bmc Genomics 2014, 15.
49. Germano G, Lamba S, Rospo G, Barault L, Magri A, Maione F, et al. Inactivation of DNA repair triggers neoantigen generation and impairs tumour growth. Nature 2017, 552(7683): 116-120.
50. Dagogo-Jack I, Shaw A T. Tumour heterogeneity and resistance to cancer therapies. Nat Rev Clin Oncol 2018, 15(2): 81-94.
51. Duan X P, Chan C, Guo N N, Han W B, Weichselbaum R R, Lin W B. Photodynamic Therapy Mediated by Nontoxic Core-Shell Nanoparticles Synergizes with Immune Checkpoint Blockade To Elicit Antitumor Immunity and Antimetastatic Effect on Breast Cancer. J Am Chem Soc 2016, 138(51): 16686-16695.
52. Fan Y C, Kuai R, Xup Y, Ochyl L J, Irvine D J, Moon J J. Immunogenic Cell Death Amplified by Co-localized Adjuvant Delivery for Cancer Immunotherapy. Nano Letters 2017, 17(12): 7387-7393.
53. Guo J F, Russell E G, Darcy R, Cotter T G, McKenna S L, Cahill M R, et al. Antibody-Targeted Cyclodextrin-Based Nanoparticles for siRNA Delivery in the Treatment of Acute Myeloid Leukemia: Physicochemical Characteristics, in Vitro Mechanistic Studies, and ex Vivo Patient Derived Therapeutic Efficacy. Mol Pharmaceut 2017, 14(3): 940-952.
54. Wang L M, Pei J, Cong Z C, Zou Y F, Sun™, Davitt F, et al. Development of anisamide-targeted PEGylated gold nanorods to deliver epirubicin for chemo-photothermal therapy in tumor-bearing mice. Int J Nanomed 2019, 14: 1817-1833.
55. Baxevanis C N, Perez S A, Papamichail M. Combinatorial treatments including vaccines, chemotherapy and monoclonal antibodies for cancer therapy. Cancer Immunol Immun 2009, 58(3): 317-324.
56. Zhao X, Yang K N, Zhao R F, Ji T J, Wang X C, Yang X, et al. Inducing enhanced immunogenic cell death with nanocarrier-based drug delivery systems for pancreatic cancer therapy. Biomaterials 2016, 102: 187-197.
57. Lu J Q, Liu X S, Liao Y P, Salazar F, Sun B B, Jiang W, et al. Nano-enabled pancreas cancer immunotherapy using immunogenic cell death and reversing immunosuppression. Nature Communications 2017, 8.
58. Banerjee R, Tyagi P, Li S, Huang L. Anisamide-targeted stealth liposomes: a potent carrier for targeting doxorubicin to human prostate cancer cells. Int J Cancer 2004, 112(4): 693-700.
59. Luan X, Rahme K, Cong Z, Wang L, Zou Y, He Y, et al. Anisamide-targeted PEGylated gold nanoparticles designed to target prostate cancer mediate: Enhanced systemic exposure of siRNA, tumour growth suppression and a synergistic therapeutic response in combination with paclitaxel in mice. Eur J Pharm Biopharm 2019, 137: 56-67.
60. Guo J, Ogier J R, Desgranges S, Darcy R, O'Driscoll C. Anisamide-targeted cyclodextrin nanoparticles for siRNA delivery to prostate tumours in mice. Biomaterials 2012, 33(31): 7775-7784.
61. Liu Q, Chen F, Hou L, Shen L, Zhang X, Wang D, et al. Nanocarrier-Mediated Chemo-Immunotherapy Arrested Cancer Progression and Induced Tumor Dormancy in Desmoplastic Melanoma. ACS Nano 2018, 12(8): 7812-7825.
62. Chen Y, Song W, Shen L, Qiu N, Hu M, Liu Y, et al. Vasodilator Hydralazine Promotes Nanoparticle Penetration in Advanced Desmoplastic Tumors. ACS Nano 2019, 13(2): 1751-1763.
63. Shen L, Li J, Liu Q, Song W, Zhang X, Tiruthani K, et al. Local Blockade of Interleukin 10 and C-X-C Motif Chemokine Ligand 12 with Nano-Delivery Promotes Antitumor Response in Murine Cancers. ACS Nano 2018, 12(10): 9830-9841.
64. Miao L, Li J, Liu Q, Feng R, Das M, Lin C M, et al. Transient and Local Expression of Chemokine and Immune Checkpoint Traps To Treat Pancreatic Cancer. ACS Nano 2017, 11(9): 8690-8706.
65. Allegra, C. J.; Yothers, G.; O'Connell, M. J.; Sharif, S.; Petrelli, N. J.; Lopa, S. H.; Wolmark, N. Bevacizumab in Stage II-III Colon Cancer: 5-Year Update of the National Surgical Adjuvant Breast and Bowel Project C-08 Trial. J Clin Oncol 2013, 31, 359-364.
66. Qin, S.; Bai, Y.; Lim, H. Y.; Thongprasert, S.; Chao, Y.; Fan, J.; Yang, T. S.; Bhudhisawasdi, V.; Kang, W. K.; Zhou, Y.; Lee, J. H.; Sun, Y. Randomized, Multicenter, Open-Label Study of Oxaliplatin Plus Fluorouracil/Leucovorin Versus Doxorubicin as Palliative Chemotherapy in Patients with Advanced Hepatocellular Carcinoma from Asia. J Clin Oncol 2013, 31, 3501-3508.
67. Douillard, J. Y.; Siena, S.; Cassidy, J.; Tabernero, J.; Burkes, R.; Barugel, M.; Humblet, Y.; Bodoky, G.; Cunningham, D.; Jassem, J.; Rivera, F.; Kocakova, I.; Ruff, P.; Blasinska-Morawiec, M.; Smakal, M.; Canon, J. L.; Rother, M.; Oliner, K. S.; Wolf, M.; Gansert, J. Randomized, Phase Iii Trial of Panitumumab with Infusional Fluorouracil, Leucovorin, and Oxaliplatin (Folfox4) Versus Folfox4 Alone as First-Line Treatment in Patients with Previously Untreated Metastatic Colorectal Cancer: The Prime Study. J Clin Oncol 2010, 28, 4697-4705.
68. Tournigand, C.; Cervantes, A.; Figer, A.; Lledo, G.; Flesch, M.; Buyse, M.; Mineur, L.; Carola, E.; Etienne, P. L.; Rivera, F.; Chirivella, I.; Perez-Staub, N.; Louvet, C.; Andre, T.; Tabah-Fisch, I.; de Gramont, A. Optimoxl: A Randomized Study of Folfox4 or Folfox7 with Oxaliplatin in a Stop-and-Go Fashion in Advanced Colorectal Cancer—a Gercor Study. J Clin Oncol 2006, 24, 394-400.
69. Andre, T.; Boni, C.; Mounedji-Boudiaf, L.; Navarro, M.; Tabernero, J.; Hickish, T.; Topham, C.; Zaninelli, M.; Clingan, P.; Bridgewater, J.; Tabah-Fisch, I.; de Gramont, A.; Investigators, M. Oxaliplatin, Fluorouracil, and Leucovorin as Adjuvant Treatment for Colon Cancer. New Engl J Med 2004, 350, 2343-2351.
70. Majumder, J.; Taratula, O.; Minko, T. Nanocarrier-Based Systems for Targeted and Site Specific Therapeutic Delivery. Adv Drug Deliv Rev 2019, 144, 57-77.
71. Guo, J.; Yu, Z.; Das, M.; Huang, L. Nano Codelivery of Oxaliplatin and Folinic Acid Achieves Synergistic Chemo-Immunotherapy with 5-Fluorouracil for Colorectal Cancer and Liver Metastasis. ACS Nano 2020, 14, 5075-5089.
72. Guo, J.; Ogier, J. R.; Desgranges, S.; Darcy, R.; O'Driscoll, C. Anisamide-Targeted Cyclodextrin Nanoparticles for Sirna Delivery to Prostate Tumours in Mice. Biomaterials 2012, 33, 7775-7784.
73. Shen, L.; Li, J.; Liu, Q.; Song, W.; Zhang, X.; Tiruthani, K.; Hu, H.; Das, M.; Goodwin, T. J.; Liu, R.; Huang, L. Local Blockade of Interleukin 10 and C-X-C Motif Chemokine Ligand 12 with Nano-Delivery Promotes Antitumor Response in Murine Cancers. ACS Nano 2018, 12, 9830-9841.
74. Song, W.; Shen, L.; Wang, Y.; Liu, Q.; Goodwin, T. J.; Li, J.; Dorosheva, O.; Liu, T.; Liu, R.; Huang, L. Synergistic and Low Adverse Effect Cancer Immunotherapy by Immunogenic Chemotherapy and Locally Expressed Pd-L1 Trap. Nat Commun 2018, 9, 2237.
75. Luan, X.; Rahme, K.; Cong, Z.; Wang, L.; Zou, Y.; He, Y.; Yang, H.; Holmes, J. D.; O'Driscoll, C. M.; Guo, J. Anisamide-Targeted Pegylated Gold Nanoparticles Designed to Target Prostate Cancer Mediate: Enhanced Systemic Exposure of Sirna, Tumour Growth Suppression and a Synergistic Therapeutic Response in Combination with Paclitaxel in Mice. Eur J Pharm Biopharm 2019, 137, 56-67.
76. Lanzavecchia, A.; Sallusto, F. Regulation of T Cell Immunity by Dendritic Cells. Cell 2001, 106, 263-266.
77. Guermonprez, P.; Valladeau, J.; Zitvogel, L.; Thery, C.; Amigorena, S. Antigen Presentation and T Cell Stimulation by Dendritic Cells. Annu Rev Immunol 2002, 20, 621-667.
78. Kroemer, G.; Galluzzi, L.; Kepp, O.; Zitvogel, L. Immunogenic Cell Death in Cancer Therapy. Annu Rev Immunol 2013, 31, 51-72.
79. Duan, X.; Chan, C.; Han, W.; Guo, N.; Weichselbaum, R. R.; Lin, W. Immunostimulatory Nanomedicines Synergize with Checkpoint Blockade Immunotherapy to Eradicate Colorectal Tumors. Nat Commun 2019, 10, 1899.
80. Chen, Q.; Chen, J.; Yang, Z.; Xu, J.; Xu, L.; Liang, C.; Han, X.; Liu, Z. Nanoparticle-Enhanced Radiotherapy to Trigger Robust Cancer Immunotherapy. Adv Mater 2019, 31, e1802228.
81. Wang, D.; Wang, T.; Yu, H.; Feng, B.; Zhou, L.; Zhou, F.; Hou, B.; Zhang, H.; Luo, M.; Li, Y. Engineering Nanoparticles to Locally Activate T Cells in the Tumor Microenvironment. Sci Immunol 2019, 4.
82. Longley, D. B.; Harkin, D. P.; Johnston, P. G. 5-Fluorouracil: Mechanisms of Action and Clinical Strategies. Nat Rev Cancer 2003, 3, 330-338.
83. Goodwin, T. J.; Shen, L.; Hu, M.; Li, J.; Feng, R.; Dorosheva, 0.; Liu, R.; Huang, L. Liver Specific Gene Immunotherapies Resolve Immune Suppressive Ectopic Lymphoid Structures of Liver Metastases and Prolong Survival. Biomaterials 2017, 141, 260-271.
84. Yang, Y.; Hu, Y.; Wang, Y.; Li, J.; Liu, F.; Huang, L. Nanoparticle Delivery of Pooled Sirna for Effective Treatment of Non-Small Cell Lung Cancer. Mol Pharm 2012, 9, 2280-2289.
85. Zhang, Y.; Peng, L.; Mumper, R. J.; Huang, L. Combinational Delivery of C-Myc Sirna and Nucleoside Analogs in a Single, Synthetic Nanocarrier for Targeted Cancer Therapy. Biomaterials 2013, 34, 8459-8468.
86. Guo, S.; Wang, Y.; Miao, L.; Xu, Z.; Lin, C. H.; Huang, L. Turning a Water and Oil Insoluble Cisplatin Derivative into a Nanoparticle Formulation for Cancer Therapy. Biomaterials 2014, 35, 7647-7653.
87. Hu, K.; Miao, L.; Goodwin, T. J.; Li, J.; Liu, Q.; Huang, L. Quercetin Remodels the Tumor Microenvironment to Improve the Permeation, Retention, and Antitumor Effects of Nanoparticles. ACS Nano 2017, 11, 4916-4925.
88. Cheng, L.; Wang, Y.; Huang, L. Exosomes from M1-Polarized Macrophages Potentiate the Cancer Vaccine by Creating a Pro-Inflammatory Microenvironment in the Lymph Node. Mol Ther 2017, 25, 1665-1675.
89. Zhang, Y.; Schwerbrock, N. M. J.; Rogers, A. B.; Kim, W. Y.; Huang, L. Codelivery of Vegf Sirna and Gemcitabine Monophosphate in a Single Nanoparticle Formulation for Effective Treatment of Nsclc. Mol Ther 2013, 21, 1559-1569.
90. Yao, J.; Zhang, Y.; Ramishetti, S.; Wang, Y. H.; Huang, L. Turning an Antiviral into an Anticancer Drug: Nanoparticle Delivery of Acyclovir Monophosphate. Journal of Controlled Release 2013, 170, 414-420.
91. Hu, M.; Wang, Y.; Xu, L.; An, S.; Tang, Y.; Zhou, X.; Li, J.; Liu, R.; Huang, L. Relaxin Gene Delivery Mitigates Liver Metastasis and Synergizes with Check Point Therapy. Nat Commun 2019, 10, 2993.
92. Li, J.; Chen, Y. C.; Tseng, Y. C.; Mozumdar, S.; Huang, L. Biodegradable Calcium Phosphate Nanoparticle with Lipid Coating for Systemic Sirna Delivery. J Control Release 2010, 142, 416-421.
93. Saif, M. W.; Syrigos, K. N.; Katirtzoglou, N. A. S-1: A Promising New Oral Fluoropyrimidine Derivative. Expert Opin Investig Drugs 2009, 18, 335-348.
94. Juliano, R. L. The Delivery of Therapeutic Oligonucleotides. Nucleic Acids Res 2016, 44, 6518-6548.
95. Li, S.; Wang, A.; Jiang, W.; Guan, Z. Pharmacokinetic Characteristics and Anticancer Effects of 5-Fluorouracil Loaded Nanoparticles. BMC Cancer 2008, 8, 103.
96. Safwat, M. A.; Soliman, G. M.; Sayed, D.; Attia, M. A. Fluorouracil-Loaded Gold Nanoparticles for the Treatment of Skin Cancer: Development, in Vitro Characterization, and in Vivo Evaluation in a Mouse Skin Cancer Xenograft Model. Mol Pharm 2018, 15, 2194-2205.
97. Kazi, J.; Mukhopadhyay, R.; Sen, R.; Jha, T.; Ganguly, S.; Debnath, M. C. Design of 5-Fluorouracil (5-Fu) Loaded, Folate Conjugated Peptide Linked Nanoparticles, a Potential New Drug Carrier for Selective Targeting of Tumor Cells. Medchemcomm 2019, 10, 559-572.
98. Franco, R.; Cidlowski, J. A. Apoptosis and Glutathione: Beyond an Antioxidant. Cell Death Differ 2009, 16, 1303-1314.
99. Lu, S. C. Regulation of Glutathione Synthesis. Mol Aspects Med 2009, 30, 42-59.
100. Zafarullah, M.; Li, W. Q.; Sylvester, J.; Ahmad, M. Molecular Mechanisms of N-Acetylcysteine Actions. Cell Mol Life Sci 2003, 60, 6-20.
101. Wang, Y. J.; Fletcher, R.; Yu, J.; Zhang, L. Immunogenic Effects of Chemotherapy-Induced Tumor Cell Death. Genes Dis 2018, 5, 194-203.
102. Bocci, G.; Danesi, R.; Di Paolo, A. D.; Innocenti, F.; Allegrini, G.; Falcone, A.; Melosi, A.; Battistoni, M.; Barsanti, G.; Conte, P. F.; Del Tacca, M. Comparative Pharmacokinetic Analysis of 5-Fluorouracil and Its Major Metabolite 5-Fluoro-5,6-Dihydrouracil after Conventional and Reduced Test Dose in Cancer Patients. Clin Cancer Res 2000, 6, 3032-3037.
103. Suk, J. S.; Xu, Q.; Kim, N.; Hanes, J.; Ensign, L. M. Pegylation as a Strategy for Improving Nanoparticle-Based Drug and Gene Delivery. Adv Drug Deliv Rev 2016, 99, 28-51.
104. Showalter, A.; Limaye, A.; Oyer, J. L.; Igarashi, R.; Kittipatarin, C.; Copik, A. J.; Khaled, A. R. Cytokines in Immunogenic Cell Death: Applications for Cancer Immunotherapy. Cytokine 2017, 97, 123-132.
105. Liu, Y.; Guo, J.; Huang, L. Modulation of Tumor Microenvironment for Immunotherapy: Focus on Nanomaterial-Based Strategies. Theranostics 2020, 10, 3099-3117.
106. Hu, M.; Zhou, X.; Wang, Y.; Guan, K.; Huang, L. Relaxin-Folfox-II-12 Triple Combination Therapy Engages Memory Response and Achieves Long-Term Survival in Colorectal Cancer Liver Metastasis. J Control Release 2020, 319, 213-221.
107. Li, Q.; Eppolito, C.; Odunsi, K.; Shrikant, P. A. 11-12-Programmed Long-Term Cd8+ T Cell Responses Require Stat4. J Immunol 2006, 177, 7618-7625.
108. Raue, H. P.; Beadling, C.; Haun, J.; Slifka, M. K. Cytokine-Mediated Programmed Proliferation of Virus-Specific Cd8(+) Memory T Cells. Immunity 2013, 38, 131-139.
109. Kasuya, H.; Kuruppu, D. K.; Donahue, J. M.; Choi, E. W.; Kawasaki, H.; Tanabe, K. K. Mouse Models of Subcutaneous Spleen Reservoir for Multiple Portal Venous Injections to Treat Liver Malignancies. Cancer Res 2005, 65, 3823-3827.
110. Guo, J.; Huang, L. Membrane-Core Nanoparticles for Cancer Nanomedicine. Adv Drug Deliv Rev 2020.
111. van der Meel, R.; Sulheim, E.; Shi, Y.; Kiessling, F.; Mulder, W. J. M.; Lammers, T. Smart Cancer Nanomedicine. Nat Nanotechnol 2019, 14, 1007-1017.
112. Roxburgh, C. S.; Shia, J.; Vakiani, E.; Daniel, T.; Weiser, M. R. Potential Immune Priming of the Tumor Microenvironment with Folfox Chemotherapy in Locally Advanced Rectal Cancer. Oncoimmunology 2018, 7, e1435227.
113. Guan, Y.; Kraus, S. G.; Quaney, M. J.; Daniels, M. A.; Mitchem, J. B.; Teixeiro, E. Folfox Chemotherapy Ameliorates Cd8 T Lymphocyte Exhaustion and Enhances Checkpoint Blockade Efficacy in Colorectal Cancer. Front Oncol 2020, 10, 586.
114. Sinicrope, F. A.; Sargent, D. J. Molecular Pathways: Microsatellite Instability in Colorectal Cancer: Prognostic, Predictive, and Therapeutic Implications. Clin Cancer Res 2012, 18, 1506-1512.
115. Ruiz-Banobre, J.; Goel, A. DNA Mismatch Repair Deficiency and Immune Checkpoint Inhibitors in Gastrointestinal Cancers. Gastroenterology 2019, 156, 890-903.
116. Liu, Y.; Guo, J.; Huang, L. Modulation of Tumor Microenvironment for Immunotherapy: Focus on Nanomaterial-Based Strategies. Theranostics 2020, 10, 3009-3117.
117. Galon, J.; Bruni, D. Approaches to Treat Immune Hot, Altered and Cold Tumours with Combination Immunotherapies. Nat Rev Drug Discov 2019, 18, 197-218.
118. Castle, J. C.; Loewer, M.; Boegel, S.; de Graaf, J.; Bender, C.; Tadmor, A. D.; Boisguerin, V.; Bukur, T.; Sorn, P.; Paret, C.; Diken, M.; Kreiter, S.; Tureci, O.; Sahin, U. Immunomic, Genomic and Transcriptomic Characterization of Ct26 Colorectal Carcinoma. Bmc Genomics 2014, 15.
119. Germano, G.; Lamba, S.; Rospo, G.; Barault, L.; Magri, A.; Maione, F.; Russo, M.; Crisafulli, G.; Bartolini, A.; Lerda, G.; Siravegna, G.; Mussolin, B.; Frapolli, R.; Montone, M.; Morano, F.; de Braud, F.; Amirouchene-Angelozzi, N.; Marsoni, S.; D'Incalci, M.; Orlandi, A.; Giraudo, E.; Sartore-Bianchi, A.; Siena, S.; Pietrantonio, F.; Di Nicolantonio, F.; Bardelli, A. Inactivation of DNA Repair Triggers Neoantigen Generation and Impairs Tumour Growth. Nature 2017, 552, 116-120.
120. Bjornmalm, M.; Thurecht, K. J.; Michael, M.; Scott, A. M.; Caruso, F. Bridging Bio-Nano Science and Cancer Nanomedicine. ACS Nano 2017, 11, 9594-9613.
121. van der Meel, R.; Lammers, T.; Hennink, W. E. Cancer Nanomedicines: Oversold or Underappreciated? Expert Opin Drug Deliv 2017, 14, 1-5.
122. Gustaysson, B.; Carlsson, G.; Machover, D.; Petrelli, N.; Roth, A.; Schmoll, H. J.; Tveit, K. M.; Gibson, F. A Review of the Evolution of Systemic Chemotherapy in the Management of Colorectal Cancer. Clin Colorectal Cancer 2015, 14, 1-10.
123. Banerjee, R.; Tyagi, P.; Li, S.; Huang, L. Anisamide-Targeted Stealth Liposomes: A Potent Carrier for Targeting Doxorubicin to Human Prostate Cancer Cells. Int J Cancer 2004, 112, 693-700.
124. Yu, Z.; Guo, J.; Hu, M.; Gao, Y.; Huang, L. Icaritin Exacerbates Mitophagy and Synergizes with Doxorubicin to Induce Immunogenic Cell Death in Hepatocellular Carcinoma. ACS Nano 2020, 14, 4816-4828.
125. Sun, H.; Yang, W.; Tian, Y.; Zeng, X.; Zhou, J.; Mok, M. T. S.; Tang, W.; Feng, Y.; Xu, L.; Chan, A. W. H.; Tong, J. H.; Cheung, Y. S.; Lai, P. B. S.; Wang, H. K. S.; Tsang, S. W.; Chow, K. L.; Hu, M.; Liu, R.; Huang, L.; Yang, B.; Yang, P.; To, K. F.; Sung, J. J. Y.; Wong, G. L. H.; Wong, V. W. S.; Cheng, A. S. L. An Inflammatory-Ccrk Circuitry Drives Mtorc1-Dependent Metabolic and Immunosuppressive Reprogramming in Obesity-Associated Hepatocellular Carcinoma. Nat Commun 2018, 9, 5214.
126. Wrightson, W. R.; Myers, S. R.; Galandiuk, S. Hplc Analysis of 5-Fu and Fdump in Tissue and Serum. Biochem Biophys Res Commun 1995, 216, 808-813.
127. Song, W.; Tiruthani, K.; Wang, Y.; Shen, L.; Hu, M.; Dorosheva, O.; Qiu, K.; Kinghorn, K. A.; Liu, R.; Huang, L. Trapping of Lipopolysaccharide to Promote Immunotherapy against Colorectal Cancer and Attenuate Liver Metastasis. Adv Mater 2018, 30, e1805007.
128. Das, M.; Shen, L.; Liu, Q.; Goodwin, T. J.; Huang, L. Nanoparticle Delivery of Rig-I Agonist Enables Effective and Safe Adjuvant Therapy in Pancreatic Cancer. Mol Ther 2019, 27, 507-517.

That which is claimed:

1. A delivery system complex comprising,
a core comprising a complex of dihydrate(1,2-diaminocyclohexane)platinum(II)-folinic acid, wherein said core is encapsulated by a liposome.

2. The delivery system complex of claim 1, wherein said complex of dihydrate(1,2-diaminocyclohexane)platinum(II)-folinic acid has the following structure:

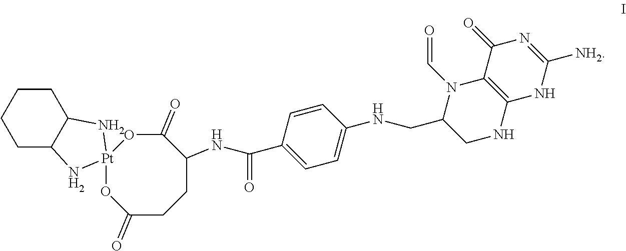

3. The delivery system complex of claim 1, wherein said liposome comprises a lipid bilayer having an inner leaflet and an outer leaflet.

4. The delivery system complex of claim 3, wherein said outer leaflet comprises a lipid-polyethylene glycol (lipid-PEG) conjugate.

5. The delivery system complex of claim 3, wherein said lipid-PEG conjugate comprises PEG in an amount between about 5 mol % to about 50 mol % of total surface lipid.

6. The delivery system complex of claim 5, wherein said lipid-PEG conjugate comprises a PEG molecule having a molecular weight of about 2000 g/mol.

7. The delivery system complex of claim 6, wherein said lipid-PEG conjugate comprises a 1,2-distearoyl-sn-glycero-3-phosphoethanolamine-N-carboxy-polyethylene glycol₂₀₀₀ (DSPE-PEG₂₀₀₀).

8. The delivery system complex of claim 2, wherein said outer leaflet comprises a targeting ligand, thereby forming a targeted delivery system complex, wherein said targeting ligand targets said targeted delivery system complex to a targeted cell.

9. The delivery system complex of claim 1, wherein said delivery system complex has a diameter of about 50 nm to about 900 nm.

10. The delivery system complex of claim 1, wherein said delivery system complex has an average diameter of about 120 nm.

11. The delivery system complex of claim 3, wherein said outer leaflet comprises a cationic lipid.

12. The delivery system complex of claim 11, wherein said cationic lipid is DOTAP.

13. The delivery system complex of claim 11, wherein said inner leaflet comprises an amphiphilic lipid.

14. The delivery system complex of claim 13, wherein the amphiphilic lipid is DOPA.

15. The delivery system complex of claim 3, wherein said outer leaflet comprises a targeting ligand.

16. The delivery system complex of claim 15, wherein said targeting ligand is aminoethyl anisamide.

17. The delivery system complex of claim 1, wherein said liposome comprises an inner leaflet comprising DOPA, an outer leaflet comprising DOTAP, cholesterol, DSPE-PEG and DSPE-PEG conjugated with aminoethyl anisamide.

18. A method of making the delivery system complex of claim 1, said method comprising:
a) preparing a precipitate of dihydrate(1,2-diaminocyclohexane)platinum(II) ([Pt(DACH)(H₂O)₂]²⁺, and folinic acid;
b) contacting said precipitate with an amphiphilic lipid to stabilize;
c) contacting the stabilized precipitate with a cationic lipid to prepare said delivery system complex.

19. A method of treating cancer comprising, administering to a subject an effective amount of the delivery system complex of claim 1.

20. The method of claim 19, further comprising administering a second active agent before, after or concurrently with said delivery system complex.

21. The method of claim 20, wherein said second active agent is an antimetabolite chemotherapeutic drug or a monoclonal antibody.

22. The method of claim 21, wherein said antimetabolite chemotherapeutic drug is 5-fluorouracil.

23. The method of claim 21, wherein said monoclonal antibody is anti-PD-L1 antibody.

24. The method of claim 19, wherein said cancer is colorectal cancer.

25. The method of claim 21, wherein said antimetabolite chemotherapeutic drug is a second delivery system complex comprising:
a core comprising an anti-metabolite complex, said anti-metabolite complex comprising a 5-fluorouracil active metabolite,
wherein said core is encapsulated by a liposome.

26. The method of claim 25, wherein said anti-metabolite complex is a precipitate of (NH₄)₂HPO₄-5-Fluoro-2′-deoxyuridine 5′-monophosphate (FdUMP).

27. A delivery system complex comprising,
a core comprising an anti-metabolite complex, said anti-metabolite complex comprising a 5-fluorouracil active metabolite,
wherein said core is encapsulated by a liposome.

28. The delivery system complex of claim 27, wherein said anti-metabolite complex is a precipitate.

29. The delivery system complex of claim 27, wherein said anti-metabolite complex is a precipitate prepared from CaCl₂, (NH₄)₂HPO₄ and 5-Fluoro-2′-deoxyuridine 5′-monophosphate (FdUMP).

30. The delivery system complex of claim 27, wherein the 5-fluorouracil active metabolite is 5-Fluoro-2′-deoxyuridine 5′-monophosphate.

31. The delivery system complex of claim 27, wherein said liposome comprises a lipid bilayer having an inner leaflet and an outer leaflet.

32. The delivery system complex of claim 31, wherein said outer leaflet comprises a lipid-polyethylene glycol (lipid-PEG) conjugate.

33. The delivery system complex of claim 32, wherein said lipid-PEG conjugate comprises PEG in an amount between about 5 mol % to about 50 mol % of total surface lipid.

34. The delivery system complex of claim 32, wherein said lipid-PEG conjugate comprises a PEG molecule having a molecular weight of about 2000 g/mol.

35. The delivery system complex of claim 32, wherein said lipid-PEG conjugate comprises a 1,2-distearoyl-sn-glycero-3-phosphoethanolamine-N-carboxy-polyethylene glycol₂₀₀₀ (DSPE-PEG₂₀₀₀).

36. The delivery system complex of claim 31, wherein said outer leaflet comprises a targeting ligand, thereby forming a targeted delivery system complex, wherein said targeting ligand targets said targeted delivery system complex to a targeted cell.

37. The delivery system complex of claim 27, wherein said delivery system complex has a diameter of about 50 nm to about 900 nm.

38. The delivery system complex of claim 27, wherein said delivery system complex has an average diameter of about 120 nm.

39. The delivery system complex of claim 31, wherein said outer leaflet comprises a cationic lipid.

40. The delivery system complex of claim 39, wherein said cationic lipid is DOTAP.

41. The delivery system complex of claim 31, wherein said inner leaflet comprises an amphiphilic lipid.

42. The delivery system complex of claim 41, wherein the amphiphilic lipid is DOPA.

43. The delivery system complex of claim 31, wherein said outer leaflet comprises a targeting ligand.

44. The delivery system complex of claim 43, wherein said targeting ligand is aminoethyl anisamide.

45. The delivery system complex of claim 27, wherein said liposome comprises an inner leaflet comprising DOPA, an outer leaflet comprising DOTAP, cholesterol, DSPE-PEG and DSPE-PEG conjugated with aminoethyl anisamide.

46. A method of making the delivery system complex of claim 27, said method comprising:
a) preparing a precipitate by contacting CaCl₂, (NH₄)₂HPO₄, and 5-fluorouracil active metabolite;
b) contacting said precipitate with an amphiphilic lipid to stabilize;
c) contacting the stabilized precipitate with a cationic lipid to prepare said delivery system complex.

47. A method of treating cancer comprising, administering to a subject an effective amount of the delivery system complex of claim 27.

48. A method of treating cancer comprising, administering to a subject a combination of:
an effective amount of the delivery system complex of claim 1;
an effective amount of the delivery system complex of claim 27; and
an effective amount of an anti-PD-L1 antibody.

49. The method of claim 19, further comprising administering a second active agent that is an antimetabolite chemotherapeutic drug before, after or concurrently with said delivery system complex, and administering a third agent that is a monoclonal antibody before, after or concurrently with said delivery system complex.

50. The method of claim 49, wherein said antimetabolite chemotherapeutic drug is 5-fluorouracil.

51. The method of claim 49, wherein said monoclonal antibody is anti-PD-L1 antibody.

52. A delivery system complex comprising, a first type of stabilized single-lipid layer core comprising an anti-metabolite complex, a second type of stabilized single-lipid layer core comprising the compound of Formula I, wherein the cores are encapsulated by a polymer.

53. The delivery system complex of claim 52, wherein the polymer is selected from the group consisting of PLGA, PLGA-PEG and PLGA-PEG-AEAA.

54. The delivery system complex of claim 52, wherein the lipid is DOPA.

55. A delivery system complex comprising, a first type of stabilized single-lipid layer core comprising an anti-metabolite complex, a second type of stabilized single-lipid layer core comprising the compound of Formula I, and irinotecan (SN-38), wherein the cores and SN-38 are encapsulated by a polymer.

56. The delivery system complex of claim 55, wherein the polymer is selected from the group consisting of PLGA, PLGA-PEG and PLGA-PEG-AEAA.

57. The delivery system complex of claim 55, wherein the lipid is DOPA.

58. A method of treating cancer comprising, administering to a subject an effective amount of the delivery system complex of claim 52 or claim 55.

59. The method of treating cancer of claim 58, further comprising, administering to a subject an effective amount of an anti-PD-L1 antibody.

60. A compound having the structure: [chemical structure]
